(19) United States
(12) Patent Application Publication
(10) Pub. No.: US 2010/0156957 A1
(43) Pub. Date: Jun. 24, 2010

(54) ORGANIC COMPOUND, ANTHRACENE DERIVATIVE, AND LIGHT-EMITTING ELEMENTS, LIGHT-EMITTING DEVICES, ELECTRONIC DEVICES, AND LIGHTING DEVICES USING THE ANTHRACENE DERIVATIVE

(75) Inventors: Kaori OGITA, Atsugi (JP); Sachiko KAWAKAMI, Atsugi (JP); Tsunenori SUZUKI, Yokohama (JP); Satoshi SEO, Kawasaki (JP)

Correspondence Address: NIXON PEABODY, LLP, 401 9TH STREET, NW, SUITE 900, WASHINGTON, DC, 20004-2128, US

(73) Assignee: SEMICONDUCTOR ENERGY LABORATORY CO., LTD., Atsugi-shi (JP)

(21) Appl. No.: 12/640,654

(22) Filed: Dec. 17, 2009

(30) Foreign Application Priority Data
Dec. 19, 2008 (JP) 2008-323612

(51) Int. Cl. G09G 5/10; C07D 209/88; H01L 51/52; H05B 37/02

(52) U.S. Cl. 345/690; 548/442; 257/40; 315/363; 257/E51.018; 257/E51.026; 257/79

(57) ABSTRACT

To provide a novel anthracene derivative which exhibit blue to blue green light emission, provide a light-emitting element which emits blue to blue green light, provide a light-emitting element which emits blue to blue green light with a long lifetime, and provide a light-emitting element which emits blue to blue green light with high emission efficiency, an anthracene derivative represented by General Formula (G1) is provided. In addition, a light-emitting element which emits blue to blue green light can be obtained by using the anthracene derivatives represented by General Formula (G1). Further, a light-emitting element which emits blue to blue green light with high emission efficiency and/or high reliability can be obtained by using the anthracene derivatives represented by General Formula (G1).

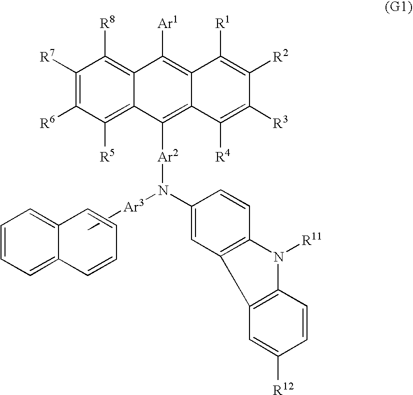

### BACKGROUND OF THE INVENTION

1. Field of the Invention

The present invention relates to organic compounds, anthracene derivatives, and light-emitting elements, light-emitting devices, lighting devices, and electronic devices in which the anthracene derivatives are used.

2. Description of the Related Art

A light-emitting element utilizing a light-emitting organic compound has a structure in which a layer including the organic compound is interposed between a pair of electrodes. This light-emitting element can be fabricated to be thin and lightweight, can emit light by application of direct current, and can response faster as compared to liquid crystals. Moreover, the light-emitting devices in which such light-emitting elements are arranged in a matrix form, i.e., passive matrix type light-emitting devices and active matrix type light-emitting devices, are superior to conventional liquid crystal displays in terms of wide viewing angle and excellent visibility. From these reasons, the light-emitting elements are expected to be applied to next-generation flat panel displays. Note that in some cases, these light-emitting elements are referred to as electroluminescent elements or EL elements.

In a light-emitting element, electrons are injected from a cathode into a layer including an organic compound interposed between a pair of electrodes, and at the same time, holes are injected from an anode into the layer including the organic compound, whereby the light-emitting element is driven. The electrons injected from the cathode and the holes injected from the anode are recombined with each other in the layer including the organic compound to form molecular excitons. The molecular excitons release energy in relaxing to a ground state. When the energy is released as light having a wavelength which corresponds to that of visible light, light emission can be observed. Excited states of organic compounds include a singlet excited state and a triplet excited state, and light can be emitted from both of the excited states.

An emission wavelength of the light-emitting element is determined by the energy gap between the ground state and the excited state formed by the recombination, i.e., a band gap. Therefore, a structure of a molecule which serves for light emission is selected or modified as appropriate, whereby desired emission color of light can be obtained. A full color light-emitting device can be manufactured by using the light-emitting elements capable of emitting light of red, blue, and green, which are three primary colors of light.

In order to manufacture a high performance full-color light-emitting device, red, blue, and green emissive light-emitting elements having a long lifetime, high emission efficiency, and excellent color purity are required. As a result of recent development of materials, excellent characteristics have been attainable for the red and green emissive light-emitting elements. However, as for a blue emissive light-emitting element, sufficient characteristics have not been obtained. For example, in Patert Document 1, a light-emitting element with a relatively long lifetime and high emission efficiency has been reported. However, in order to achieve a high performance full-color display, a longer lifetime and higher emission efficiency have been required.

### REFERENCE

### [Patert Document]

### [Patert Document 1] Japanese Published Patert Application No. 2007-91721.

### DISCLOSURE OF THE INVENTION

In view of the foregoing problems, an object of an embodiment of the present invention is to provide novel anthracene derivatives which exhibit blue to blue green light emission and a novel organic compound which is used in synthesis of the anthracene derivatives.

It is another object to provide a light-emitting element which emits blue to blue green light. It is another object to provide a light-emitting element which emits blue to blue green light with a long lifetime. It is still another object to provide a light-emitting element which emits blue to blue green light with high emission efficiency. Further, another object is to provide a light-emitting element which emits blue to blue green light with a long lifetime and high emission efficiency.

Another object is to provide a light-emitting device with reduced power consumption, and a lighting device and an electronic device in which the light-emitting element is used.

### SUMMARY OF THE INVENTION

As a result of diligent study, the inventors have found that at least one of the above problems can be solved with an anthracene derivative represented by General Formula (G1) below. One embodiment of the present invention is an anthracene derivative having a naphthyl group at the terminal of an amine skeleton. Thus, one embodiment of the present invention is an anthracene derivative represented by General Formula (G1) below.

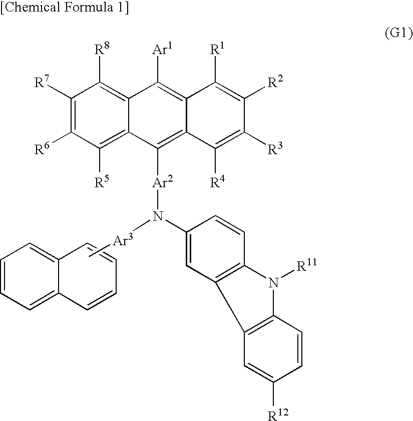

In General Formula (G1) above, Ar¹ represents a substituted or unsubstituted aryl group having 6 to 13 carbon atoms. When Ar¹ has substituents, the substituents may be bonded to each other to form a ring. Furthermore, when a carbon atom of Ar¹ has two substituents, the substituents may be bonded to each other to form a spiro ring. In General Formula (G1) above, Ar² and Ar³ each independently represent a substituted or unsubstituted arylene group having 6 to 13 carbon atoms. When Ar² and Ar³ have substituents, the substituents may be bonded to each other to form a ring. Furthermore, when a carbon atom of Ar² and Ar³ has two substituents, the substituents may be bonded to each other to form a spiro ring. In General Formula (G1) above, R¹ to R⁸ each represent either hydrogen or an alkyl group having 1 to 4 carbon atoms. In General Formula (G1) above, R¹¹ represents either an alkyl group having 1 to 4 carbon atoms or a substituted or unsubstituted aryl group having 6 to 13 carbon atoms. When R¹¹ is an aryl group having substituents, the substituents may be bonded to each other to form a ring. Furthermore, when a carbon atom of R¹¹ has two substituents, the substituents may be bonded to each other to form a spiro ring. In General Formula (G1) above, R¹² represents hydrogen, an alkyl group having 1 to 4 carbon atoms, or a substituted or unsubstituted aryl group having 6 to 13 carbon atoms. When R¹² is an aryl group having substituents, the substituents may be bonded to each other to form a ring. Furthermore, when a carbon atom of R¹² has two substituents, the substituents may be bonded to each other to form a spiro ring.

Further, an embodiment of the present invention is an anthracene derivative represented by General Formula (G1-1) below.

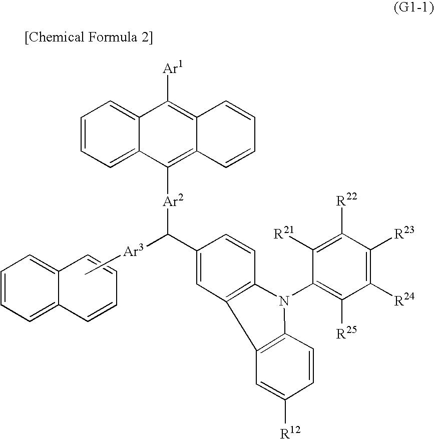

In General Formula (G1-1) above, Ar¹ represents a substituted or unsubstituted aryl group having 6 to 13 carbon atoms. When Ar¹ has substituents, the substituents may be bonded to each other to form a ring. Furthermore, when a carbon atom of Ar¹ has two substituents, the substituents may be bonded to each other to form a spiro ring. In General Formula (G1-1) above, Ar² and Ar³ each independently represent a substituted or unsubstituted arylene group having 6 to 13 carbon atoms. When Ar² and Ar³ have substituents, the substituents may be bonded to each other to form a ring. Furthermore, when a carbon atom of Ar² and Ar³ has two substituents, the substituents may be bonded to each other to form a spiro ring. In General Formula (G1-1) above, R¹² represents hydrogen, an alkyl group having 1 to 4 carbon atoms, or a substituted or unsubstituted aryl group having 6 to 13 carbon atoms. When R¹² is an aryl group having substituents, the substituents may be bonded to each other to form a ring. Furthermore, when a carbon atom of R¹² has two substituents, the substituents may be bonded to each other to form a spiro ring. In General Formula (G1-1) above, R²¹ to R²⁵ each represent hydrogen, an alkyl group having 1 to 4 carbon atoms, or a substituted or unsubstituted aryl group having 6 to 10 carbon atoms. When R²¹ to R²⁵ are aryl groups having substituents, the substituents may be bonded to each other to form a ring. Furthermore, when a carbon atom of R²¹ to R²⁵ has two substituents, the substituents may be bonded to each other to form a spiro ring.

Further, an embodiment of the present invention is an anthracene derivative represented by General Formula (G1-2) below.

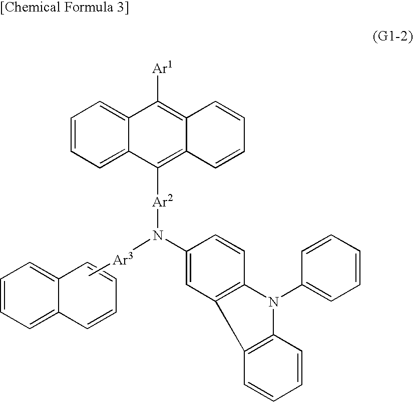

In General Formula (G1-2) above, Ar¹ represents a substituted or unsubstituted phenyl group, a 1-naphthyl group, or a 2-naphthyl group. In General Formula (G1-2) above, Ar² and Ar³ each independently represent a substituted or unsubstituted arylene group having 6 to 13 carbon atoms. When Ar² and Ar³ have substituents, the substituents may be bonded to each other to form a ring. Furthermore, when a carbon atom of Ar² and Ar³ has two substituents, the substituents may be bonded to each other to form a spiro ring.

Further, an embodiment of the present invention is an anthracene derivative represented by General Formula (G1-3) below.

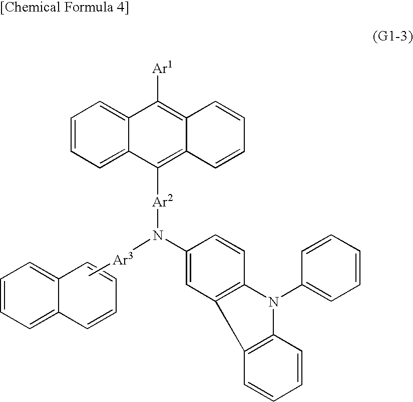

In General Formula (G1-3) above, Ar¹ represents a substituted or unsubstituted phenyl group, a 1-naphthyl group, or a 2-naphthyl group. In General Formula (G1-3) above. Ar² represents a substituted or unsubstituted arylene group having 6 to 13 carbon atoms. When Ar² has substituents, the substituents may be bonded to each other to form a ring. Furthermore, when a carbon atom of Ar² has two substituents, the substituents may be bonded to each other to form a spiro ring. In General Formula (G1-3) above, Ar^{a} represents a para-phenylene group or a biphenyl-4,4′-diyl group.

Further, an embodiment of the present invention is an anthracene derivative represented by General Formula (G1-4) below.

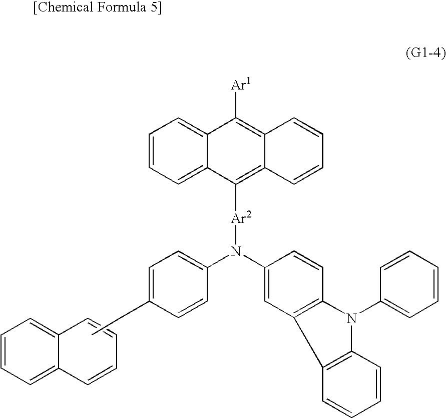

In General Formula (G1-4) above, Ar¹ represents a substituted or unsubstituted phenyl group, a 1-naphthyl group, or a 2-naphthyl group. In General Formula (G1-4) above, Ar² represents a para-phenylene group or a biphenyl-4,4′-diyl group.

Further, an embodiment of the present invention is an anthracene derivative represented by Structural Formula (101) below.

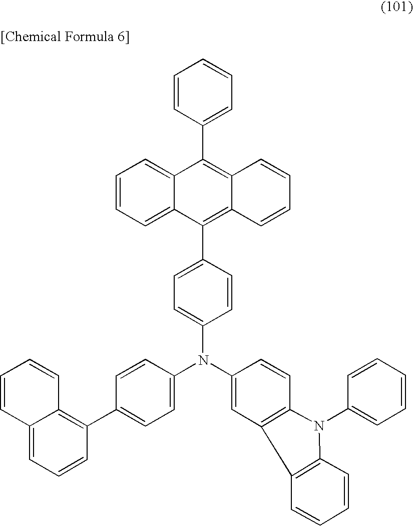

Another embodiment of the present invention is a light-emitting element including any of the above anthracene derivatives. In other words, an embodiment of the present invention is a light-emitting element including any of the above anthracene derivatives between a pair of electrodes.

Since the above anthracene derivatives have high emission efficiency, it is preferred that the anthracene derivatives be used in a light-emitting layer. Thus, one embodiment of the present invention is a light-emitting element which includes a light-emitting layer between a pair of electrodes, where the light-emitting layer includes any of the above-described anthracene derivatives.

The light-emitting element in accordance with an embodiment of the present invention obtained in this manner can realize a long lifetime, and thus, a light-emitting device (including an image display device) in which such a light-emitting element is utilized can also realize a long lifetime. Thus, an embodiment of the present invention also includes the light-emitting device, a lighting device, and an electronic device each of which uses the light-emitting element described above.

The light-emitting device in accordance with an embodiment of the present invention includes a light-emitting element including any of the above-described anthracene derivatives and a control circuit for controlling light emission from the light-emitting element. Note that the light-emitting device in this specification includes an image display device using a light-emitting element. Further, the light-emitting device includes: a module including a light-emitting element to which a connector such as an anisotropic conductive film, a tape automated bonding (TAB) tape, or a tape carrier package (TCP) is added; a module in which the top of the TAB tape or the TCP is provided with a printed wiring board; a module in which an integrated circuit (IC) is directly mounted on a light-emitting element by a chip on glass (COG) technique; and the like. Moreover, a light-emitting device used in a lighting device or the like is also included.

Further, an electronic device using the light-emitting element in accordance with an embodiment of the present invention in a display portion is also included in the scope of the present invention. Accordingly, an embodiment of the present invention is an electronic device including a display portion provided with the above-described light-emitting element and a control circuit which controls light emission of the light-emitting element.

Furthermore, since organic compounds used for the synthesis of the anthracene derivatives in accordance with an embodiment of the present invention are also novel materials, the organic compounds used for the synthesis of the anthracene derivatives in accordance with an embodiment of the present invention are also included in an embodiment of the present invention. Thus, an embodiment of the present invention is an organic compound represented by General Formula (G2) below.

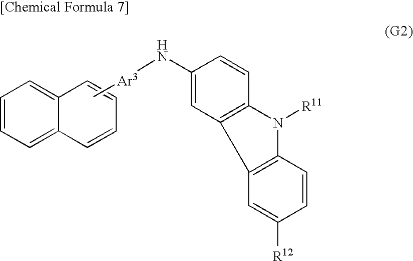

In General Formula (G2) above, Ar³ represents a substituted or unsubstituted arylene group having 6 to 13 carbon atoms. When Ar³ has substituents, the substituents may be bonded to each other to form a ring. Furthermore, when a carbon atom of Ar³ has two substituents, the substituents may be bonded to each other to form a spiro ring. In General Formula (G2) above, R¹¹ represents an alkyl group having 1 to 4 carbon atoms or a substituted or unsubstituted aryl group having 6 to 13 carbon atoms. When R¹¹ is an aryl group having substituents, the substituents may be bonded to each other to form a ring. Furthermore, when a carbon atom of R¹¹ has two substituents, the substituents may be bonded to each other to form a spiro ring. In General Formula (G2) above, R¹² represents hydrogen, an alkyl group having 1 to 4 carbon atoms, or a substituted or unsubstituted aryl group having 6 to 13 carbon atoms. When R¹² is an aryl group having substituents, the substituents may be bonded to each other to form a ring. Furthermore, when a carbon atom of R¹² has two substituents, the substituents may be bonded to each other to form a spiro ring.

Further, an embodiment of the present invention is an organic compound represented by General Formula (G2-1) below.

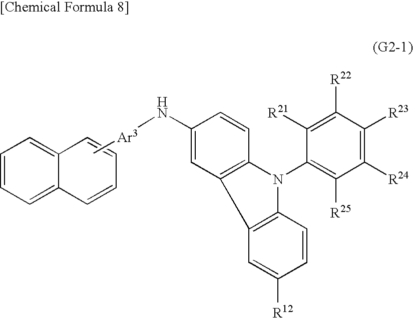

In General Formula (G2-1) above, Ar³ represents a substituted or unsubstituted arylene group having 6 to 13 carbon atoms. When Ar³ has substituents, the substituents may be bonded to each other to form a ring. Furthermore, when a carbon atom of Ar³ has two substituents, the substituents may be bonded to each other to form a spiro ring. In General Formula (G2-1) above, R¹² represents hydrogen, an alkyl group having 1 to 4 carbon atoms, or a substituted or unsubstituted aryl group having 6 to 13 carbon atoms. When R¹² is an aryl group having substituents, the substituents may be bonded to each other to form a ring. Furthermore, when a carbon atom of R¹² has two substituents, the substituents may be bonded to each other to form a spiro ring. In General Formula (G2-1) above, R² to R²⁵ each represent hydrogen, an alkyl group having 1 to 4 carbon atoms, or a substituted or unsubstituted aryl group having 6 to 10 carbon atoms. When R²¹ to R²⁵ are aryl groups having substituents, the substituents may be bonded to each other to form a ring. Furthermore, when a carbon atom of R²¹ to R²⁵ has two substituents, the substituents may be bonded to each other to form a spiro ring.

Further, an embodiment of the present invention is an organic compound represented by General Formula (G2-2) below.

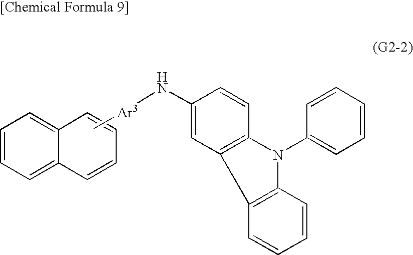

In General Formula (G2-2) above, Ar³ represents a substituted or unsubstituted arylene group having 6 to 13 carbon atoms. When Ar³ has substituents, the substituents may be bonded to each other to form a ring. Furthermore, when a carbon atom of Ar³ has two substituents, the substituents may be bonded to each other to form a spiro ring.

Further, an embodiment of the present invention is an organic compound represented by General Formula (G2-3) below.

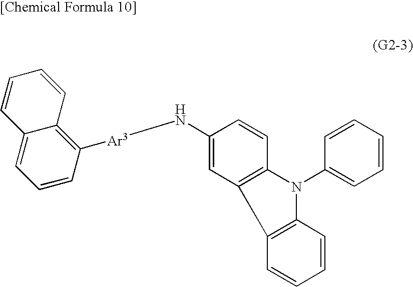

In General Formula (G2-3) above, Ar³ represents a substituted or unsubstituted arylene group having 6 to 13 carbon atoms. When Ar³ has substituents, the substituents may be bonded to each other to form a ring. Furthermore, when a carbon atom of Ar³ has two substituents, the substituents may be bonded to each other to form a spiro ring.

Further, an embodiment of the present invention is an organic compound represented by General Formula (G2-4) below.

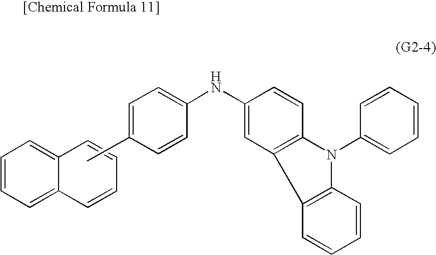

Further, an embodiment of the present invention is an organic compound represented by Structural Formula (301) below.

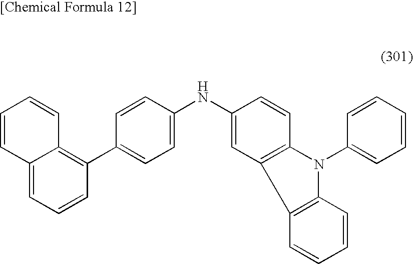

The anthracene derivatives in accordance with an embodiment of the present invention have high emission efficiency. In addition, the anthracene derivatives in accordance with an embodiment of the present invention can emit blue to blue green light. Further, the anthracene derivatives in accordance with an embodiment of the present invention are highly resistant to repetitive oxidation and reduction and are electrochemically stable.

In addition, with the use of the anthracene derivatives in accordance with an embodiment of the present invention, a light-emitting element which emits blue to blue green light can be obtained. Further, a light-emitting element which emits blue to blue green light with high emission efficiency can be obtained. Moreover, a light-emitting element which emits blue to blue green light with a long lifetime can be obtained.

Further, the use of the anthracene derivatives in accordance with an embodiment of the present invention allows the production of a light-emitting device, a lighting device, and an electronic device with reduced power consumption.

### BRIEF DESCRIPTION OF THE DRAWINGS

In the accompanying drawings:

FIGS. 1A and 1B illustrate a light emitting element in accordance with an embodiment of the present invention;

FIG. 2 illustrates a light-emitting element in accordance with an embodiment of the present invention;

FIGS. 3A and 3B illustrate a light-emitting device in accordance with an embodiment of the present invention;

FIGS. 4A and 4B illustrate a light-emitting device in accordance with an embodiment of the present invention;

FIGS. 5A to 5D illustrate electronic devices in accordance with an embodiment of the present invention;

FIG. 6 illustrates a lighting device in accordance with an embodiment of the present invention;

FIG. 7 illustrates a lighting device in accordance with an embodiment of the present invention;

FIG. 8 illustrates a lighting device in accordance with an embodiment of the present invention;

FIG. 9 illustrates a light-emitting element in accordance with an embodiment of the present invention;

FIGS. 10A and 10B show ¹H-NMR charts of N-[4-(1-naphthyl)phenyl-N-[4-(10-phenyl-9-anthryl)phenyl]-9-phenyl-9H-carbazol-3-amine (abbreviated to PCNAPA);

FIG. 11 shows an absorption spectrum of a toluene solution of N-[4-(1-naphthyl)phenyl-N-[4-(10-phenyl-9-anthryl)phenyl]-9-phenyl-9H-carbazol-3-amine (abbreviated to PCNAPA);

FIG. 12 shows an emission spectrum of a toluene solution of N-[4-(1-naphthyl)phenyl-N-[4-(10-phenyl-9-anthryl)phenyl]-9-phenyl-9H-carbazol-3-amine (abbreviated to PCNAPA);

FIG. 13 shows an absorption spectrum of a thin film of N-[4-(1-naphthyl)phenyl-N-[4-(10-phenyl-9-anthryl)phenyl]-9-phenyl-9H-carbazol-3-amine (abbreviated to PCNAPA);

FIG. 14 shows an emission spectrum of a thin film of N-[4-(1-naphthyl)phenyl-N-[4-(10-phenyl-9-anthryl)phenyl]-9-phenyl-9H-carbazol-3-amine (abbreviated to PCNAPA);

FIGS. 15A and 15B show CV charts of a DMF solution of N-[4-(1-naphthyl)phenyl-N-[4-(10-phenyl-9-anthryl)phenyl]-9-phenyl-9H-carbazol-3-amine (abbreviated to PCNAPA);

FIG. 16 shows current density-luminance characteristics of light-emitting elements manufactured in Example 2;

FIG. 17 shows voltage-luminance characteristics of light emitting elements manufactured in Example 2;

FIG. 18 shows luminance-current efficiency characteristics of light-emitting elements manufactured in Example 2;

FIG. 19 shows emission spectra of light-emitting elements manufactured in Example 2; and

FIG. 20 shows results of driving tests of light-emitting elements manufactured in Example 2.

### DETAILED DESCRIPTION OF THE INVENTION

Hereinafter, the embodiments of the present invention will be described in detail with reference to the accompanying drawings. However, the present invention is not limited to the following description, and various changes and modifications for the modes and details thereof will be apparent to those skilled in the art unless such changes and modifications depart from the spirit and scope of the invention. Therefore, the present invention is not construed as being limited to the description of the embodiments and examples below.

### Embodiment 1

In this embodiment, a novel anthracene derivative and an organic compound used for the synthesis of the anthracene derivative is described.

The anthracene derivative described in this embodiment is represented by General Formula (G1).

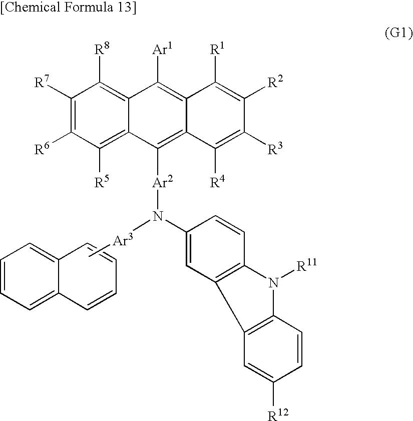

In General Formula (G1) above, Ar¹ represents a substituted or unsubstituted aryl group having 6 to 13 carbon atoms. When Ar¹ has substituents, the substituents may be bonded to each other to form a ring. Furthermore, when a carbon atom of Ar¹ has two substituents, the substituents may be bonded to each other to form a spiro ring. In General Formula (G1) above, Ar² and Ar³ each independently represent a substituted or unsubstituted arylene group having 6 to 13 carbon atoms. When Ar² and Ar³ have substituents, the substituents may be bonded to each other to form a ring. Furthermore, when a carbon atom of Ar² and Ar³ has two substituents, the substituents may be bonded to each other to form a spiro ring. In General Formula (G1) above, R¹ to R⁸ each represent either hydrogen or an alkyl group having 1 to 4 carbon atoms. In General Formula (G1) above, R¹¹ represents either an alkyl group having 1 to 4 carbon atoms or a substituted or unsubstituted aryl group having 6 to 13 carbon atoms. When R¹ is an aryl group having substituents, the substituents may be bonded to each other to form a ring. Furthermore, when a carbon atom of R¹¹ has two substituents, the substituents may be bonded to each other to form a spiro ring. In General Formula (G1) above, R¹² represents hydrogen, an alkyl group having 1 to 4 carbon atoms, or a substituted or unsubstituted aryl group having 6 to 13 carbon atoms. When R¹² is an aryl group having substituents, the substituents may be bonded to each other to form a ring. Furthermore, when a carbon atom of R¹² has two substituents, the substituents may be bonded to each other to form a spiro ring.

The anthracene derivative represented by General Formula (G1) in this embodiment includes a naphthyl group at the terminal of an amine skeleton. Specifically, in General Formula (G1), the anthracene derivative includes the naphthyl group which is directly bonded to Ar³. Introduction of the naphthyl group at the terminal of the amine skeleton allows the anthracene derivative represented by General Formula (G1) to efficiently emit light and exhibit high electrochemical stability.

In General Formula (G1) above, structures represented by Structural Formulae (I-1) to (1-20) can be given as an example of an aryl group represented by Ar¹.

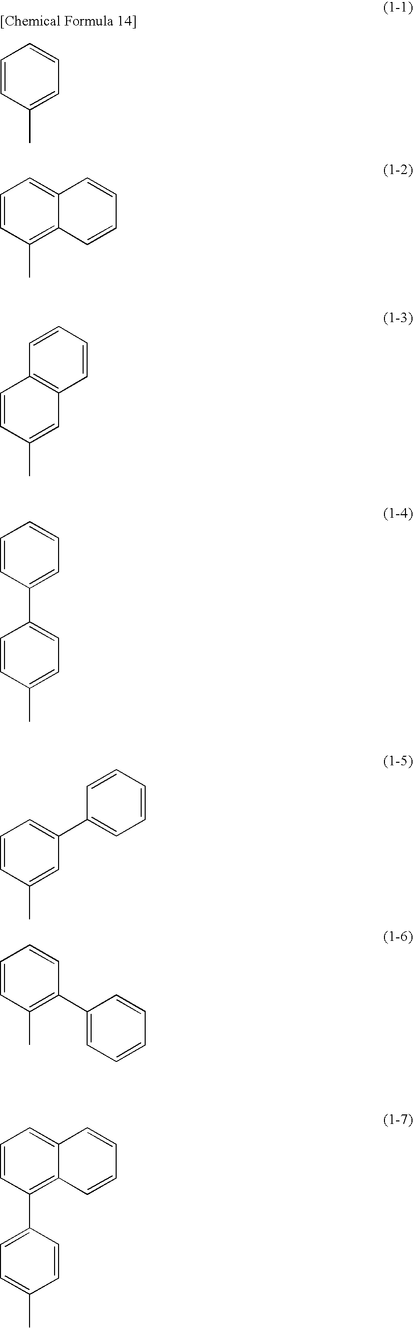

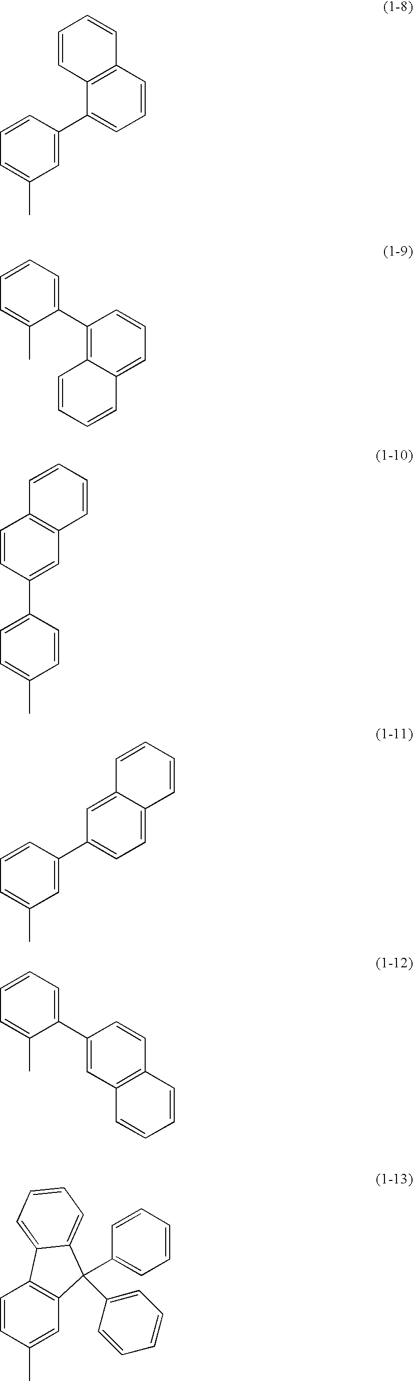

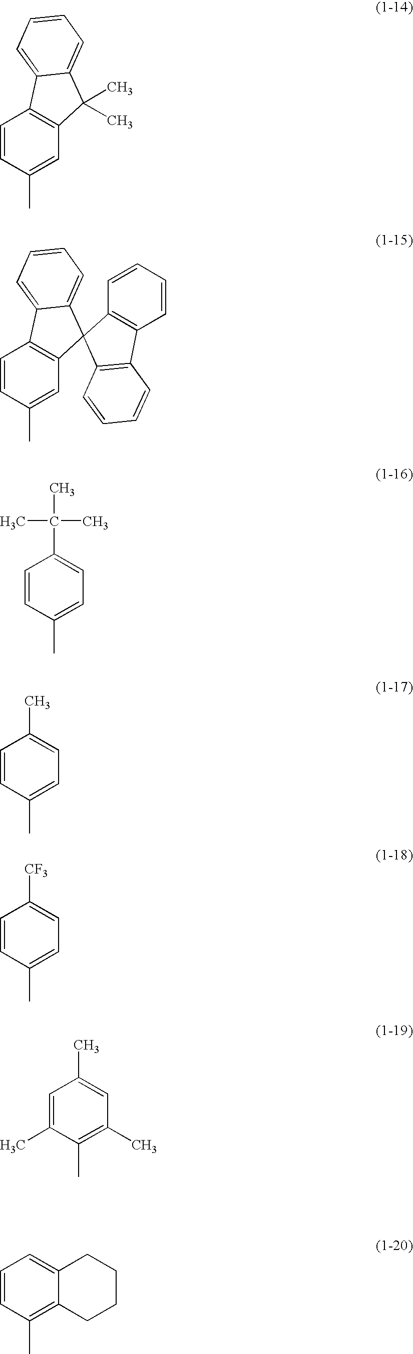

Further, in General Formula (G1) above, structures represented by Structural Formulae (2-1) to (2-11) can be given as an example of an arylene group represented by Ar² and Ar³.

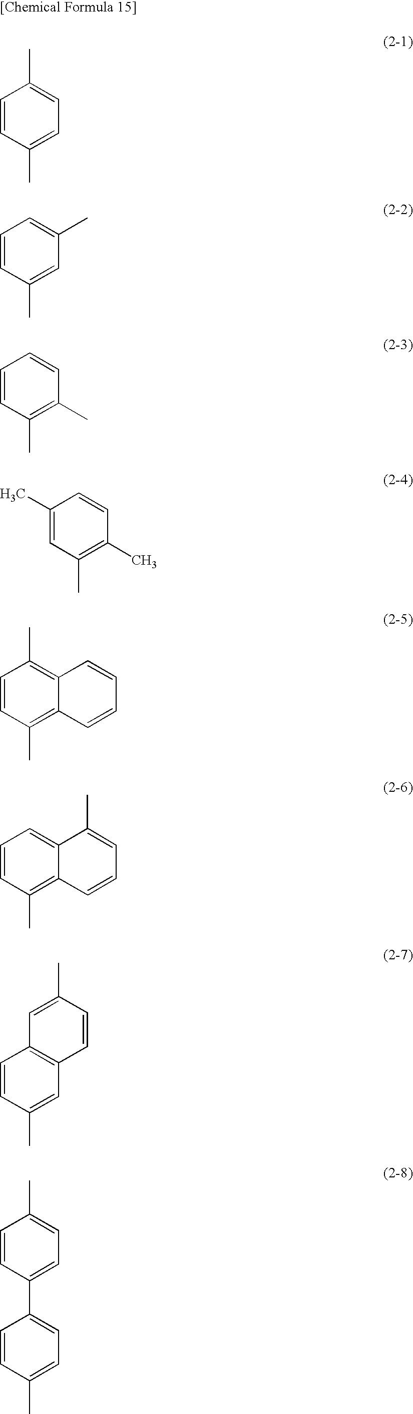

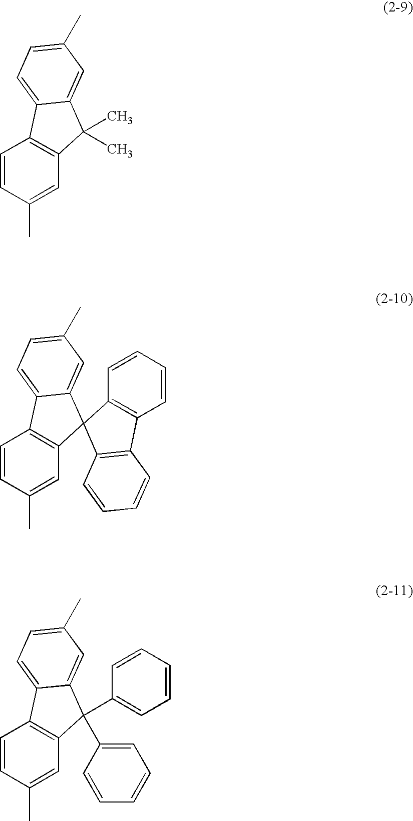

Further, in General Formula (G1) above, structures represented by Structural Formulae (3-1) to (3-28) can be given as an example of an arylene group represented by R¹¹.

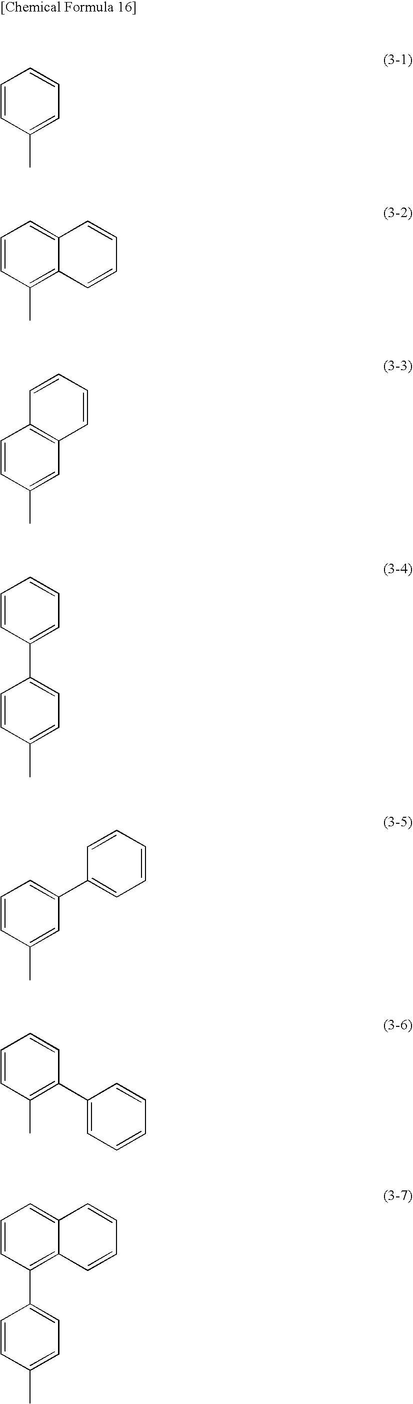

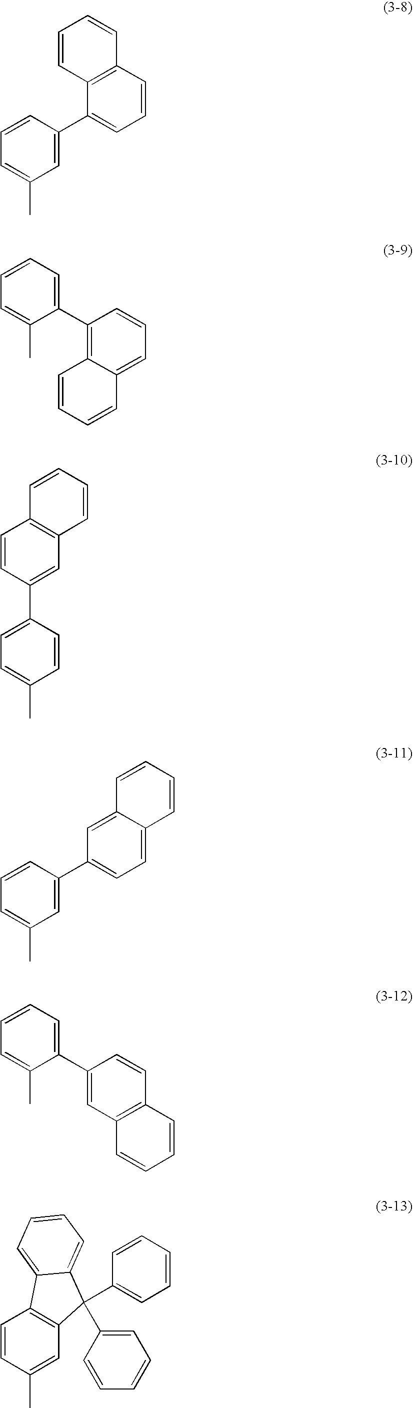

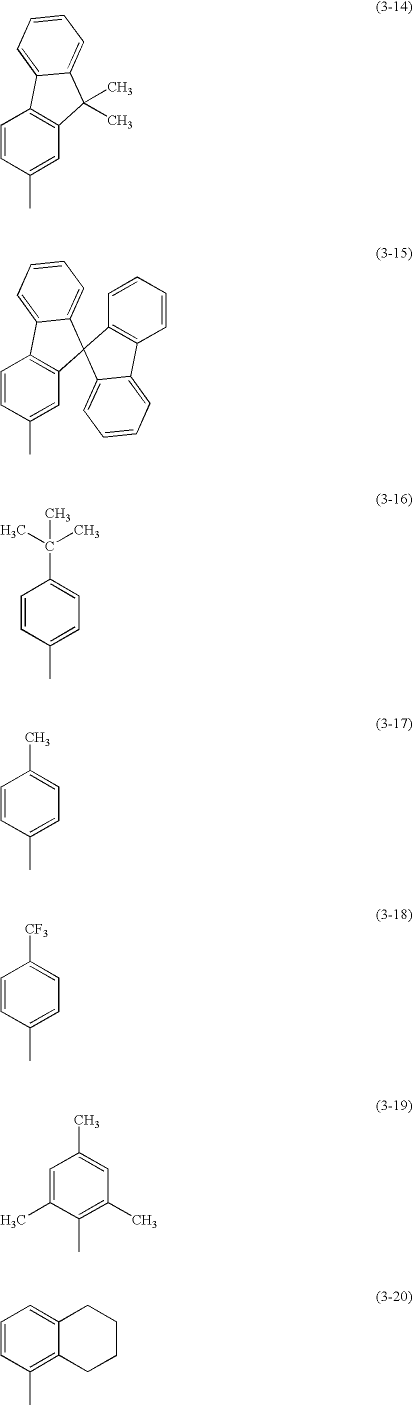

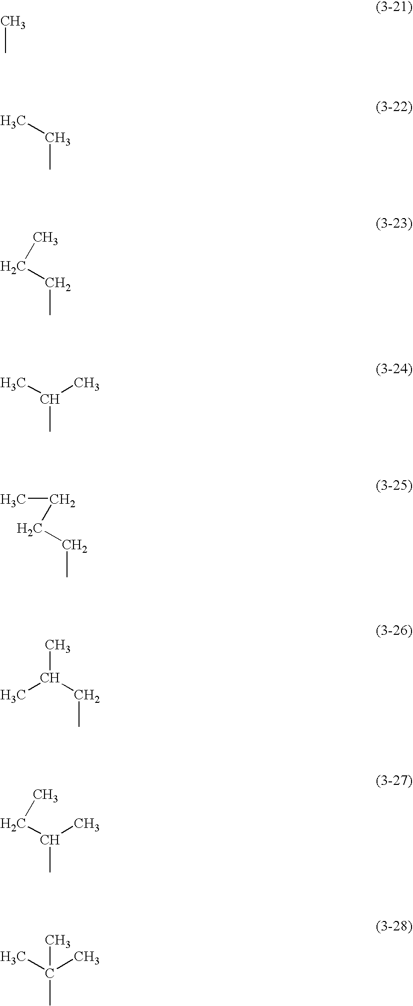

Further, in General Formula (G1) above, structures represented by Structural Formulae (4-0) to (4-28) can be given as an example of an arylene group represented by R¹².

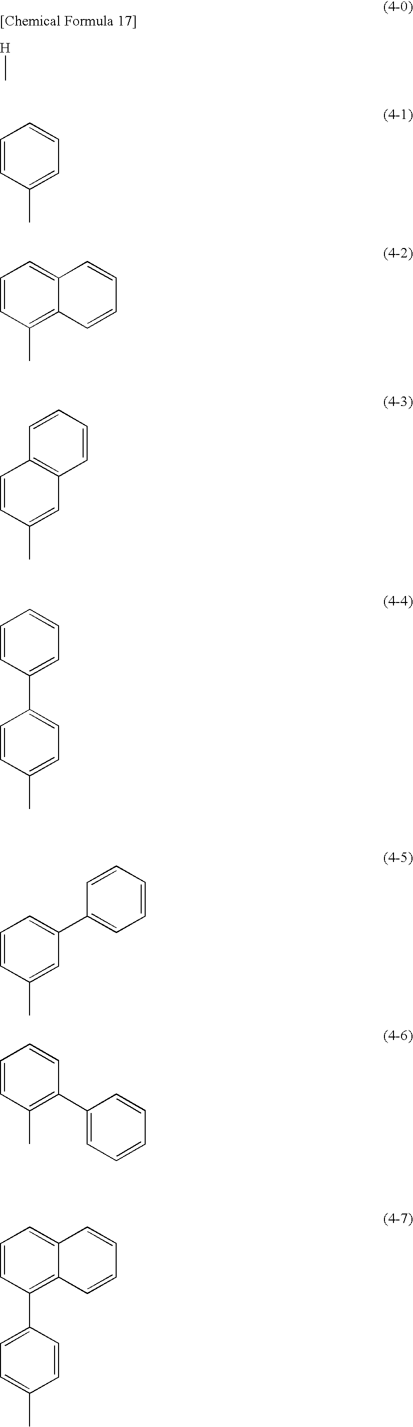

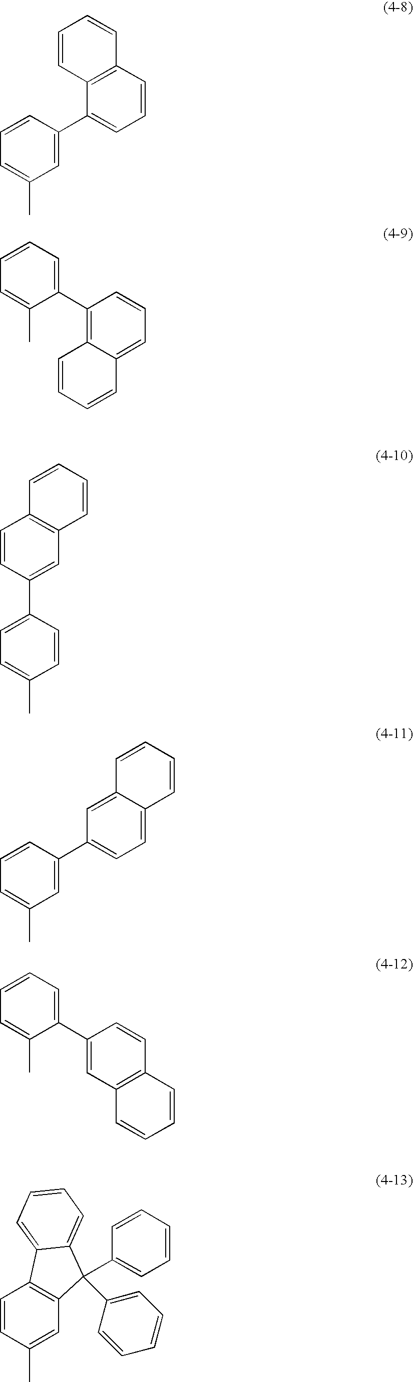

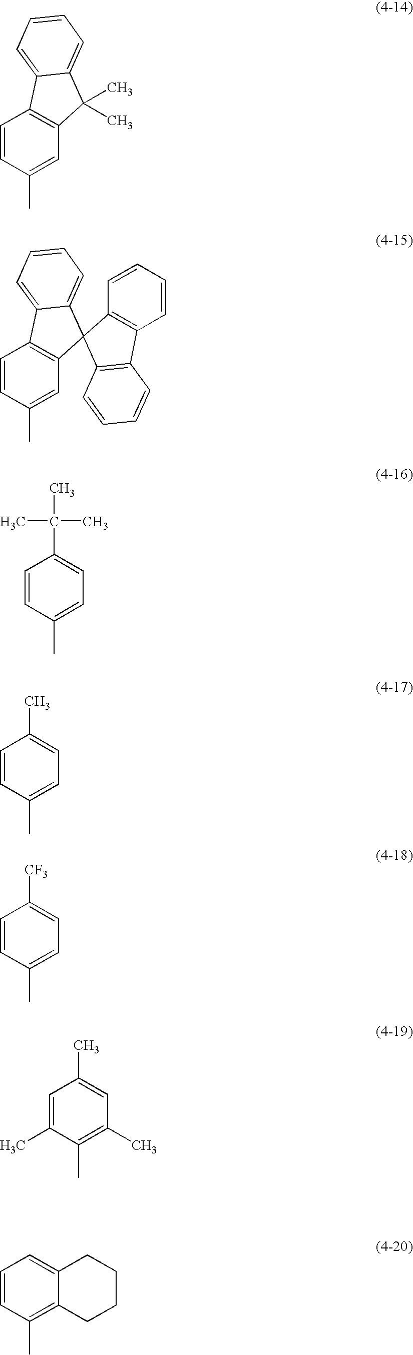

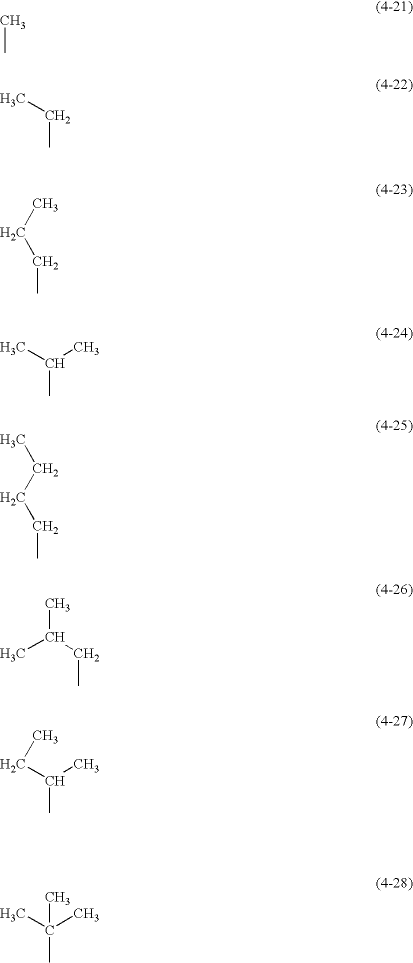

Specific examples of such anthracene derivatives can be anthracene derivatives represented by Structural Formulae (101) to (162) and by Structural Formulae (201) to (262) below. However, the present invention is not limited to these examples.

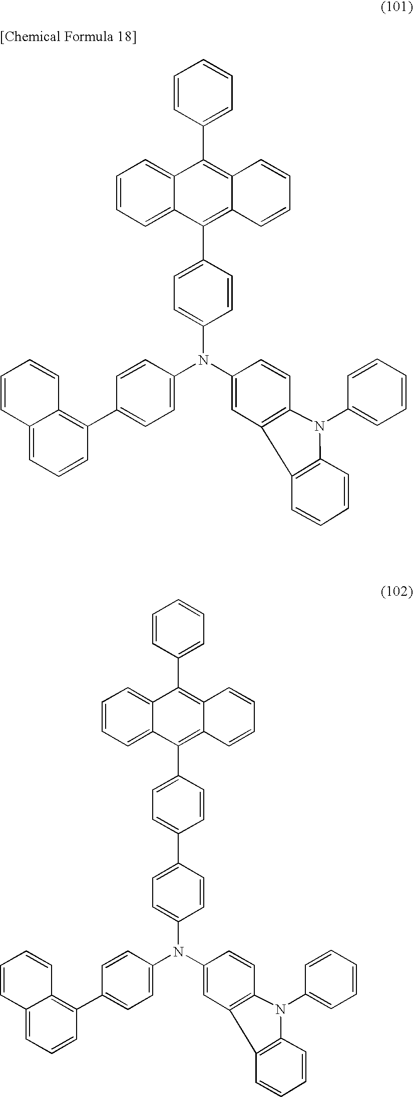

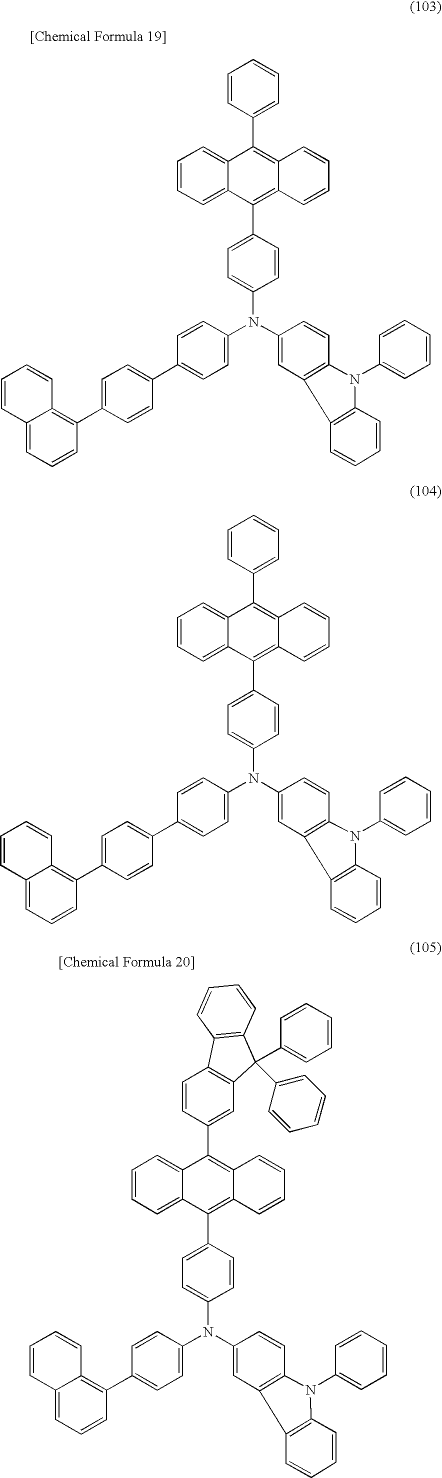

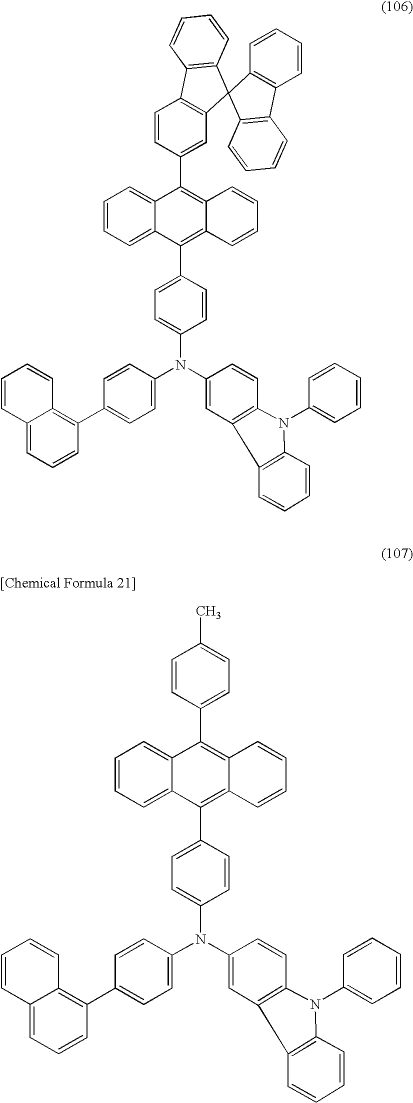

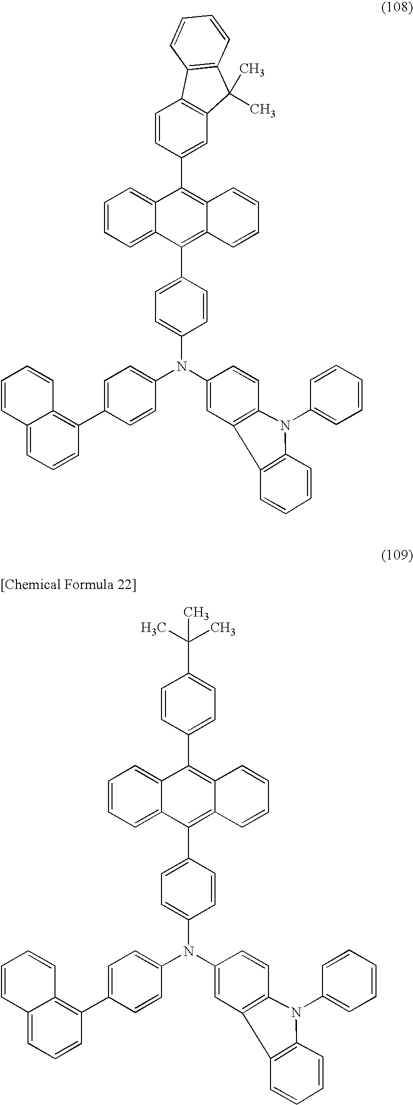

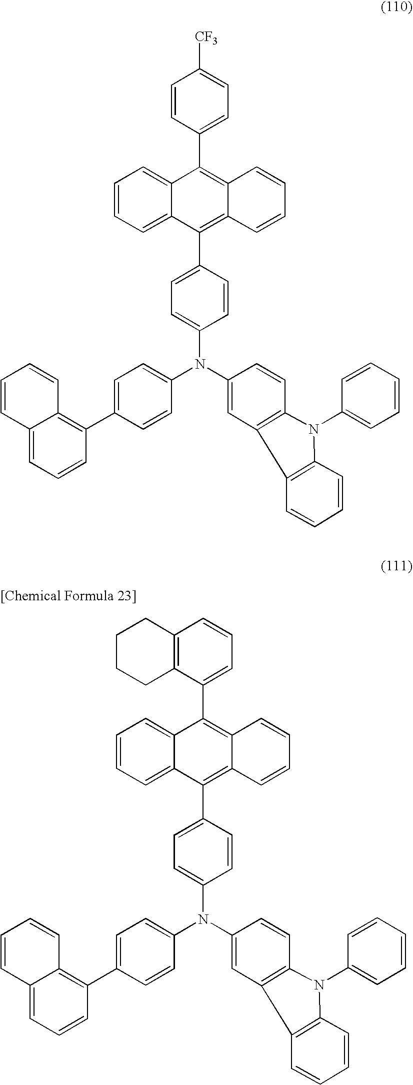

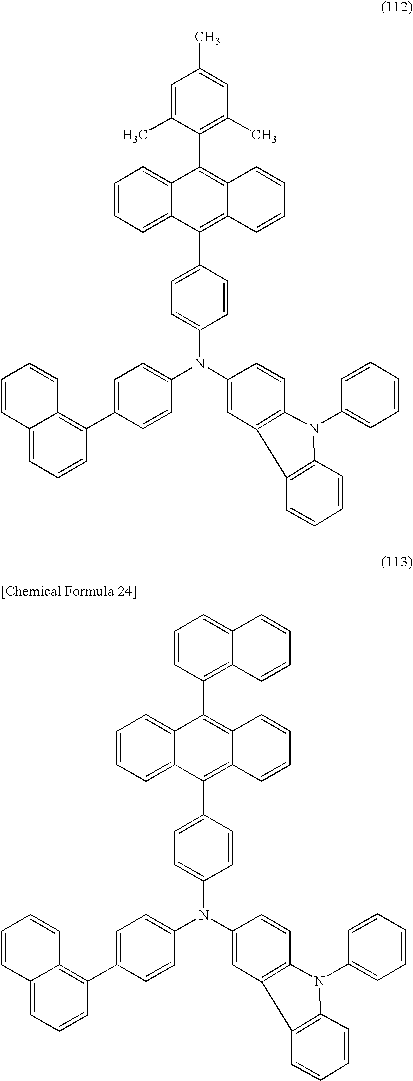

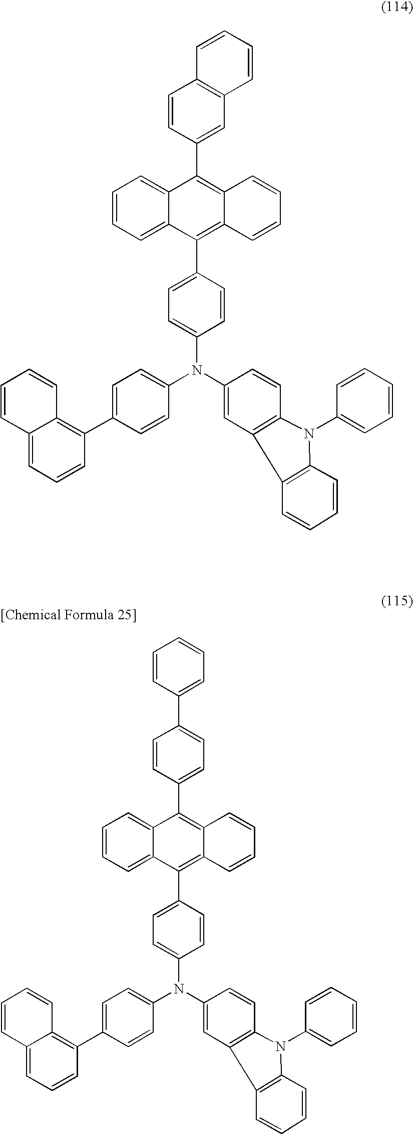

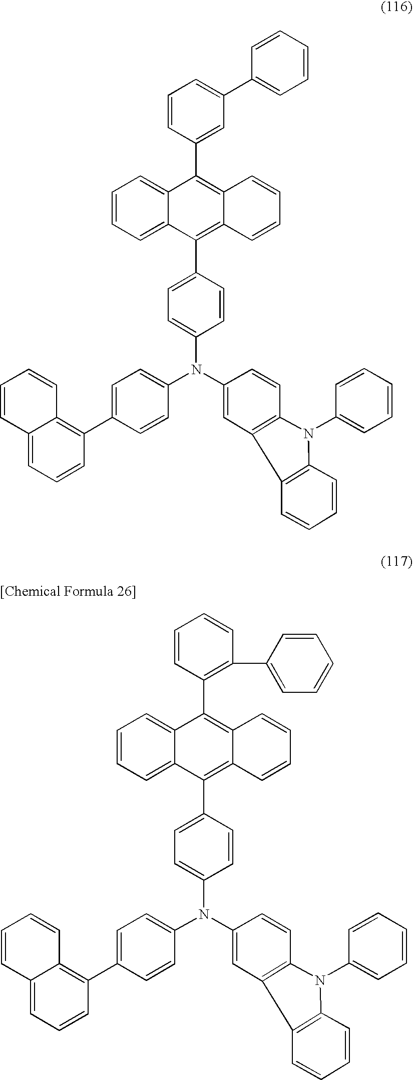

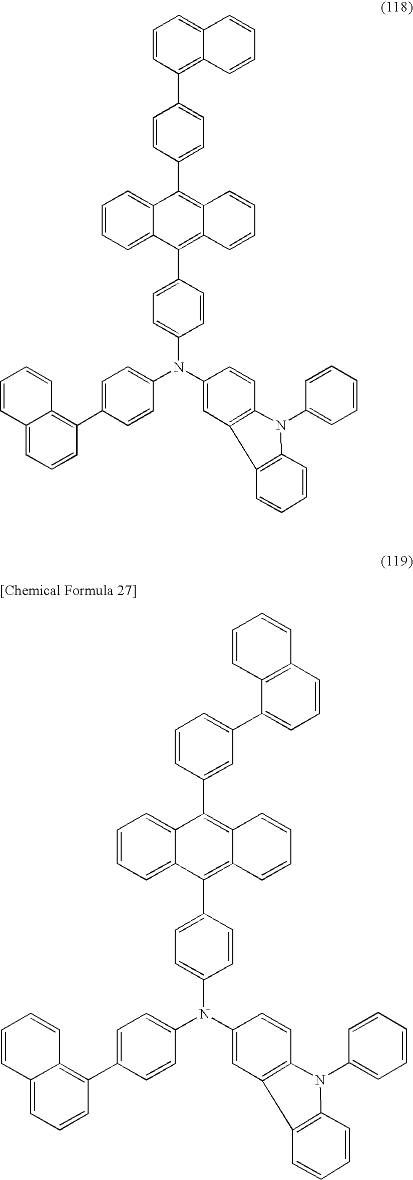

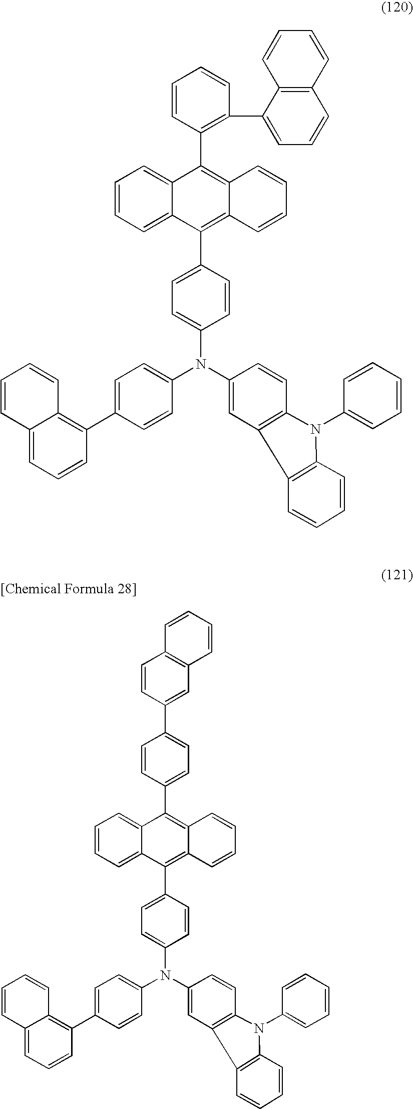

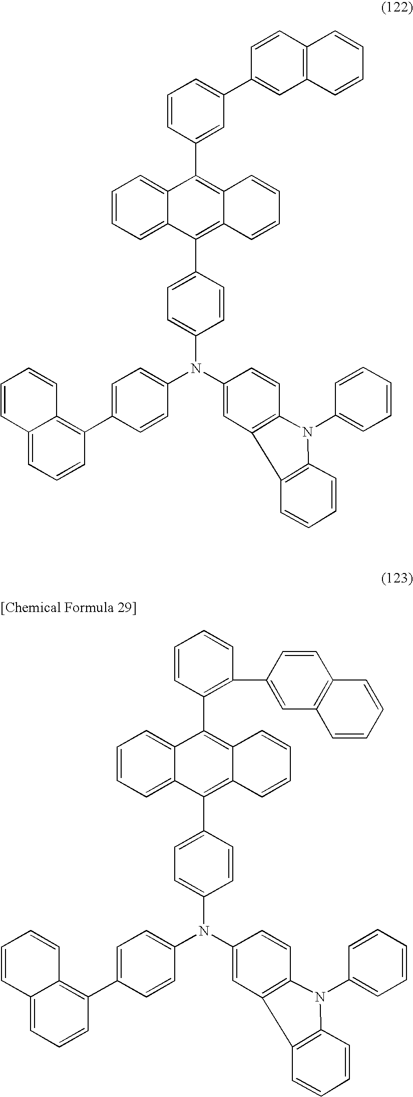

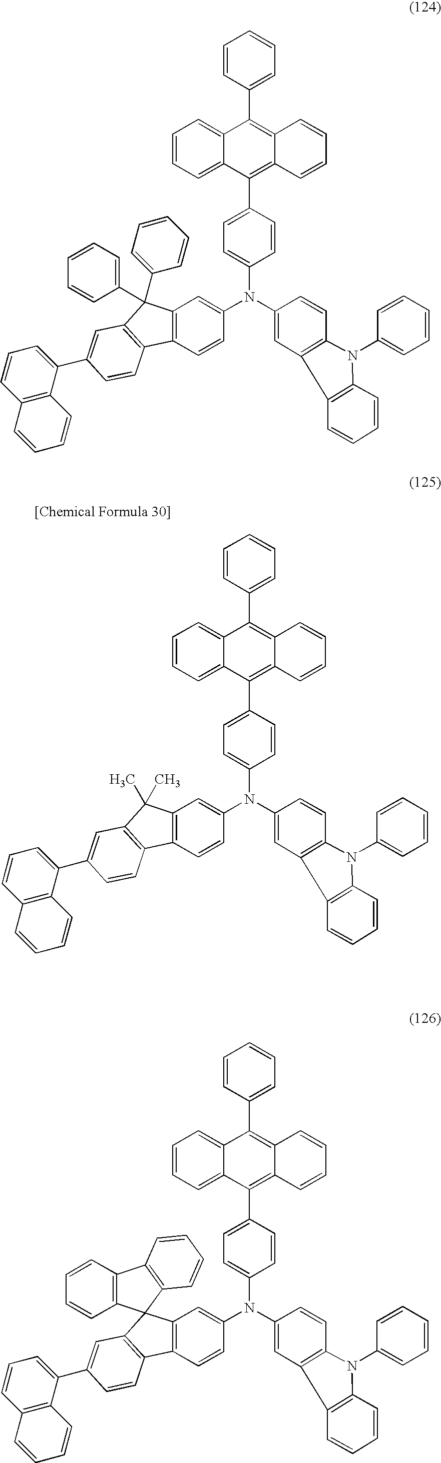

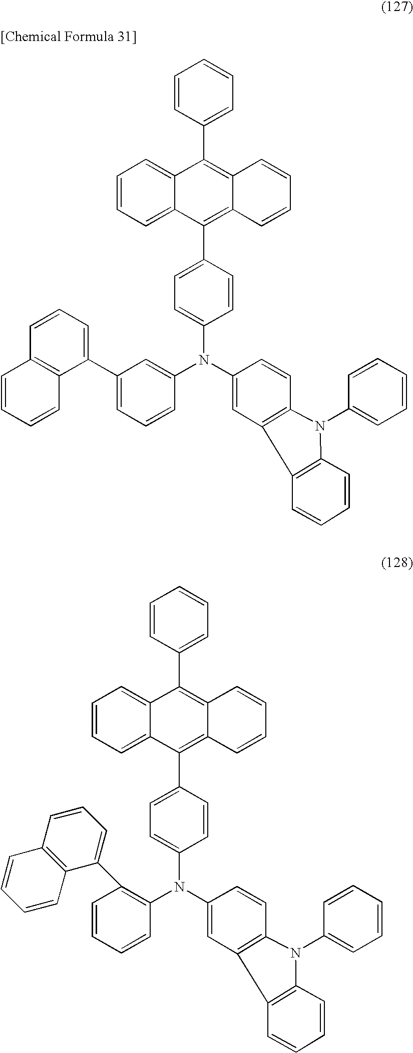

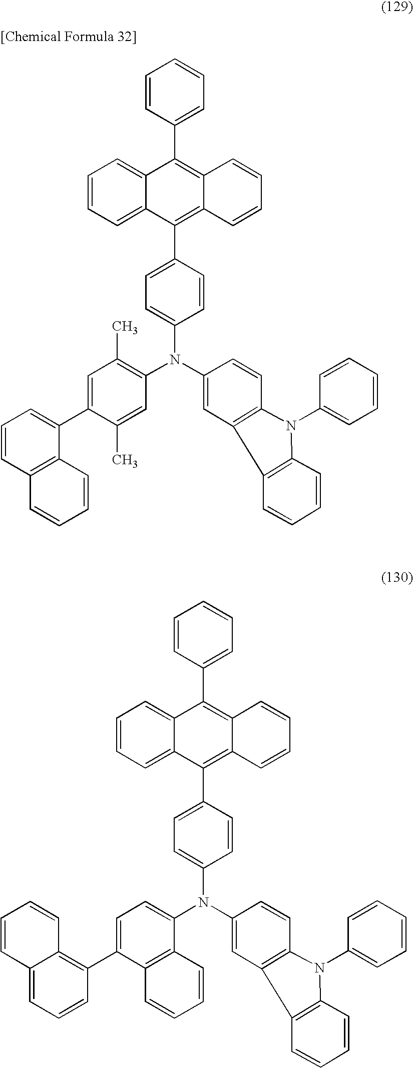

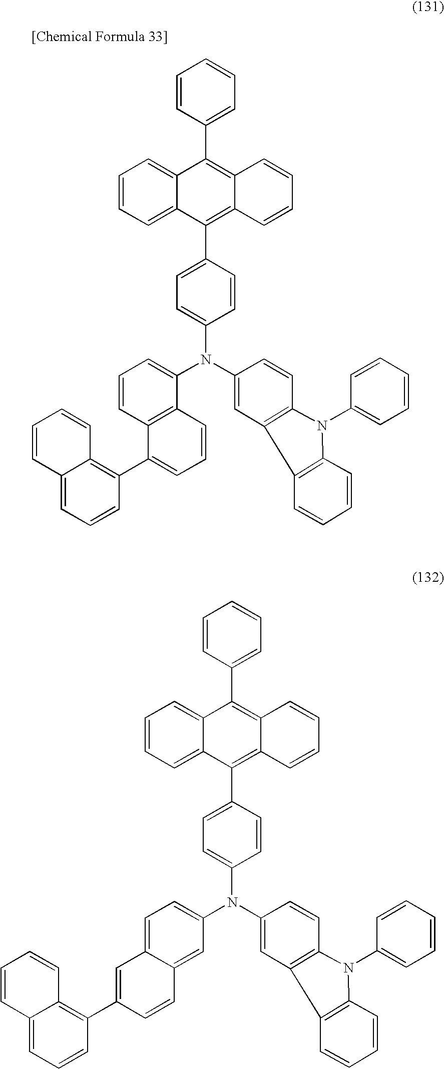

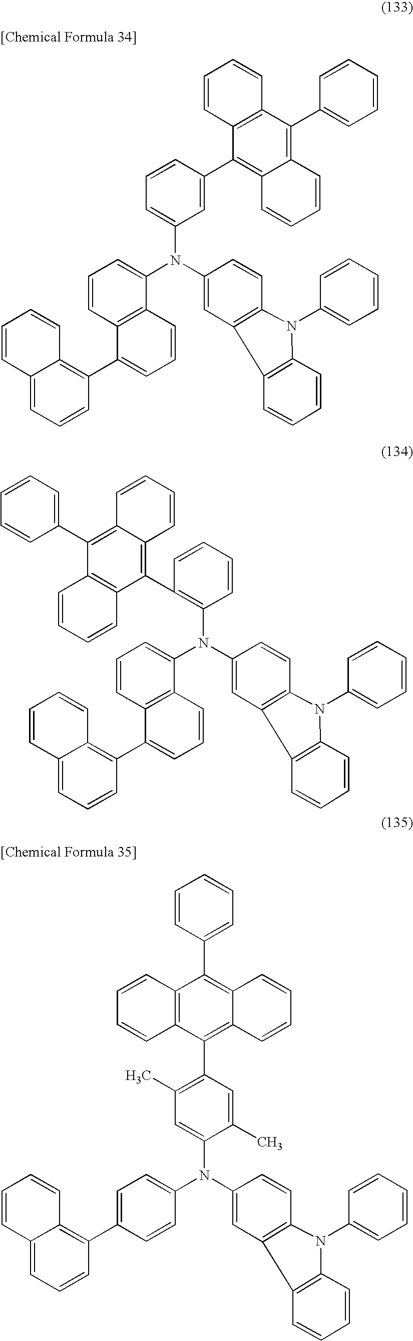

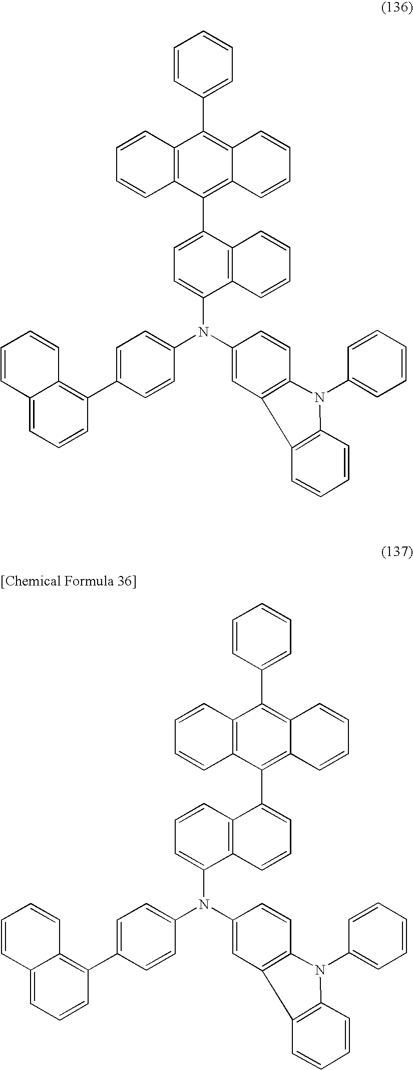

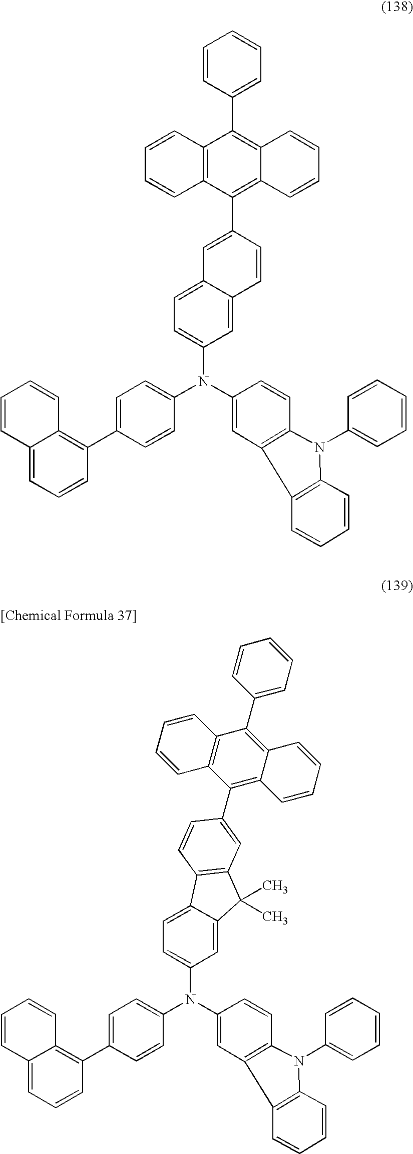

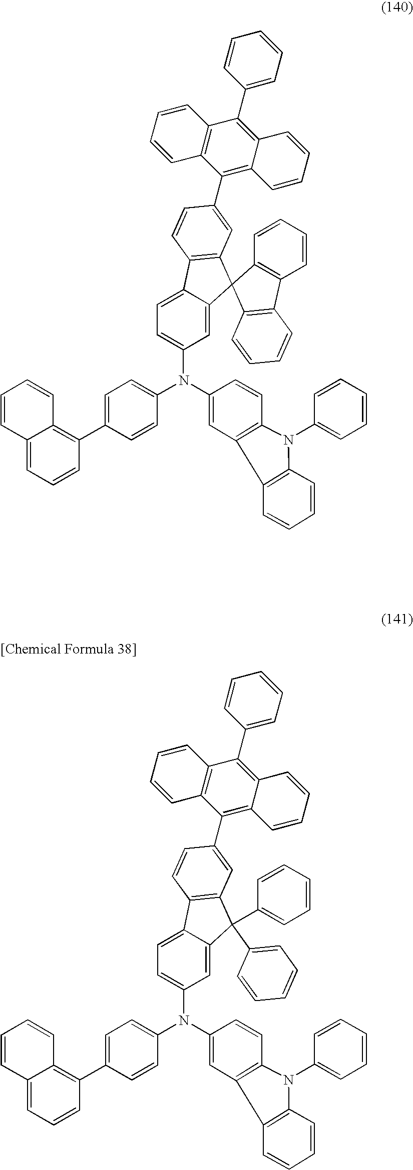

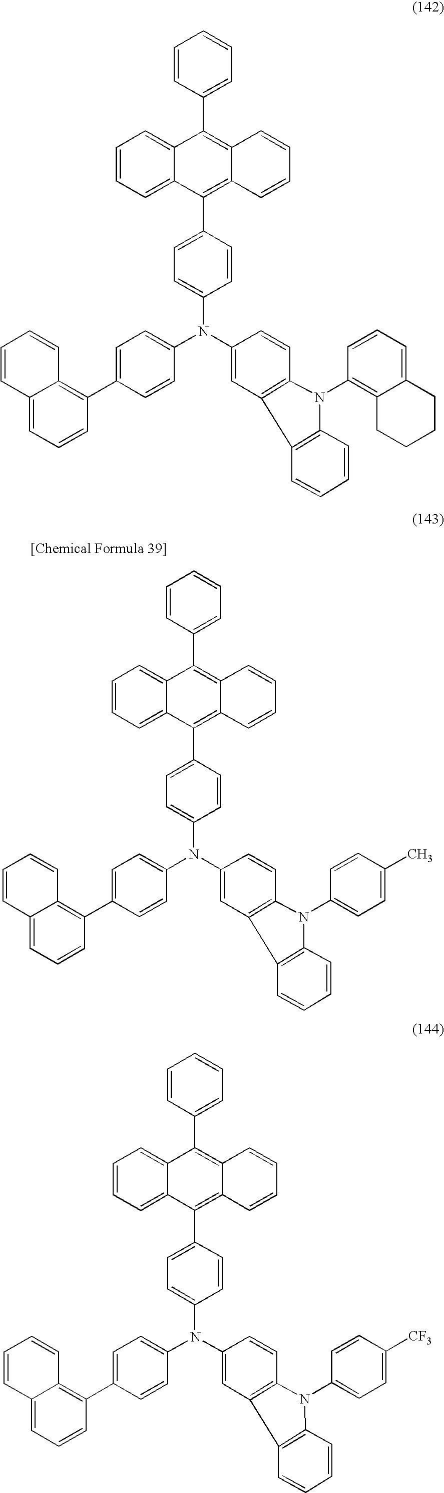

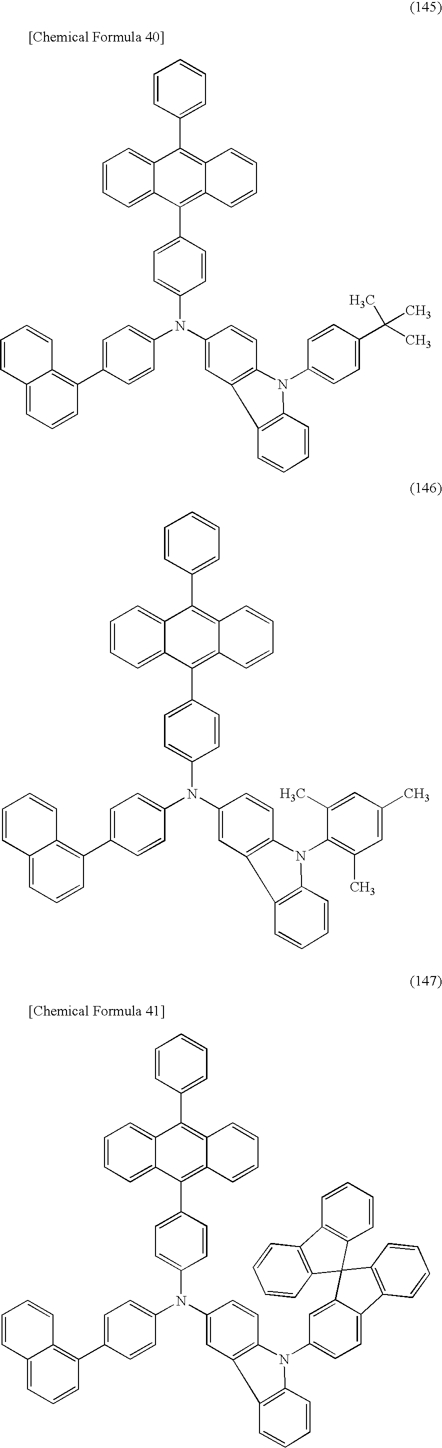

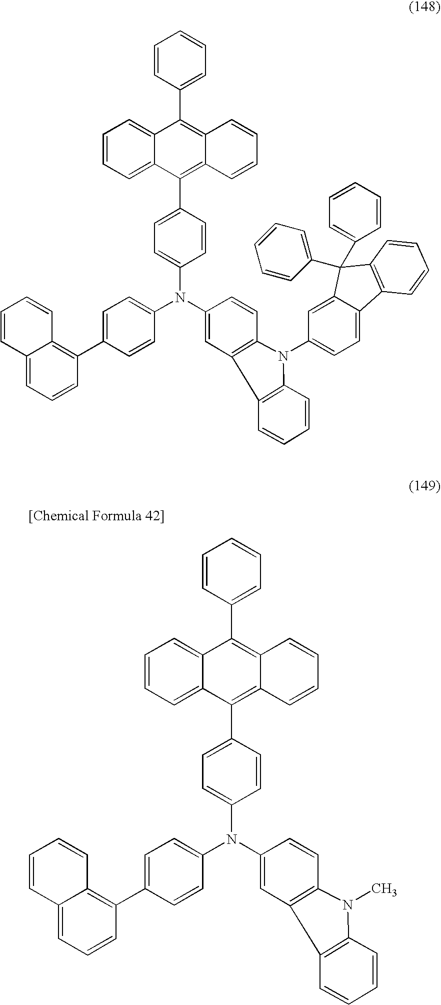

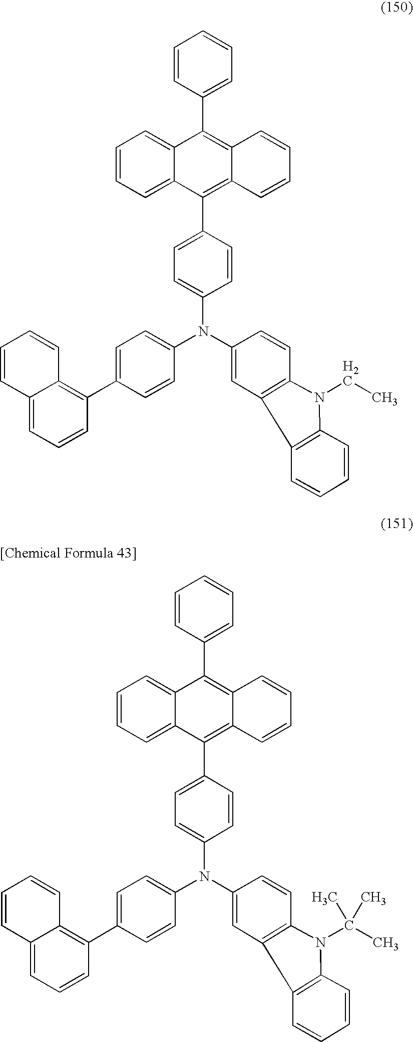

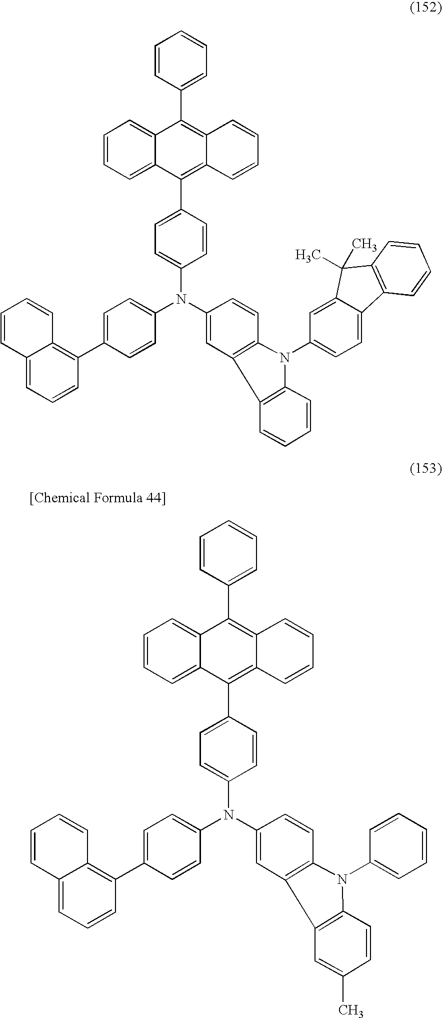

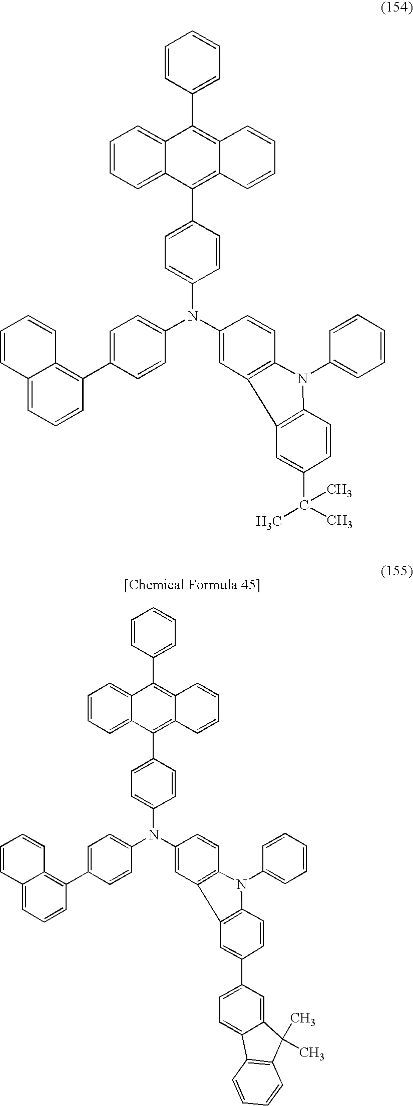

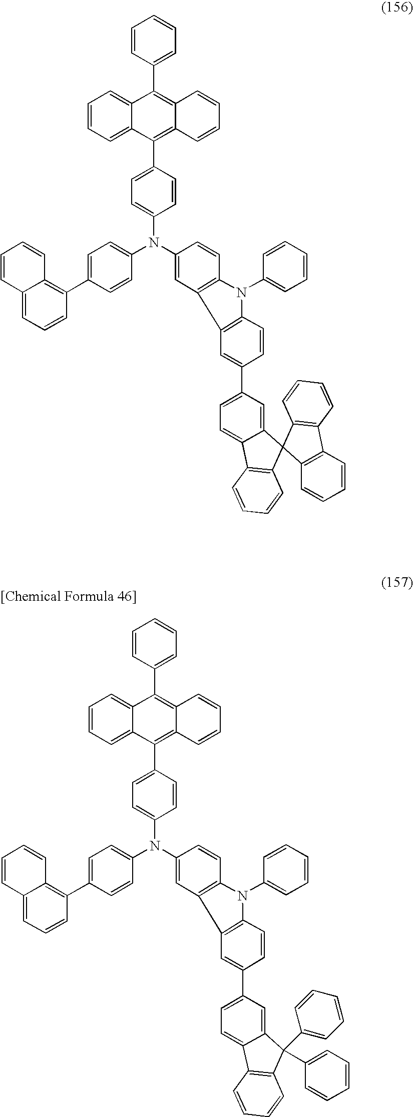

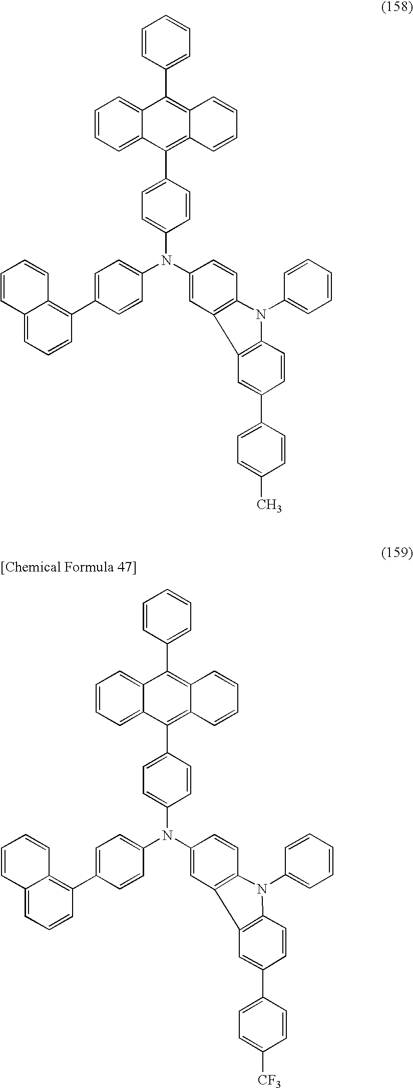

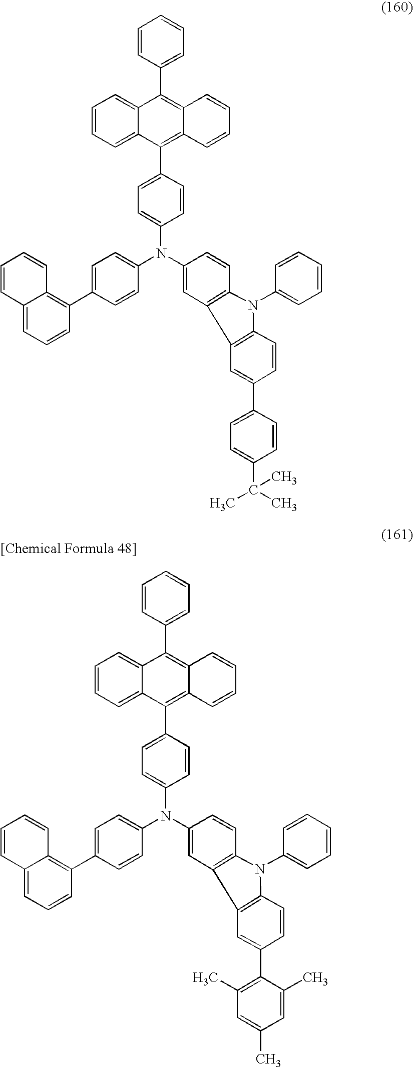

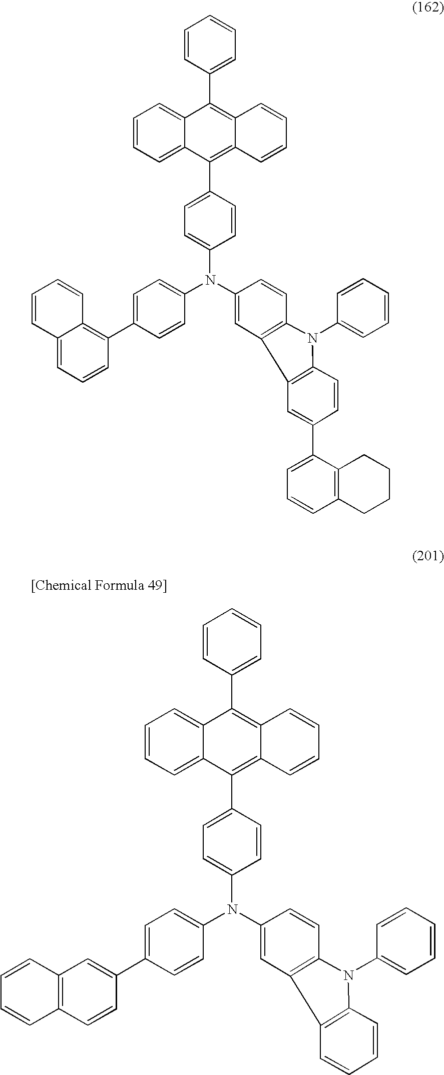

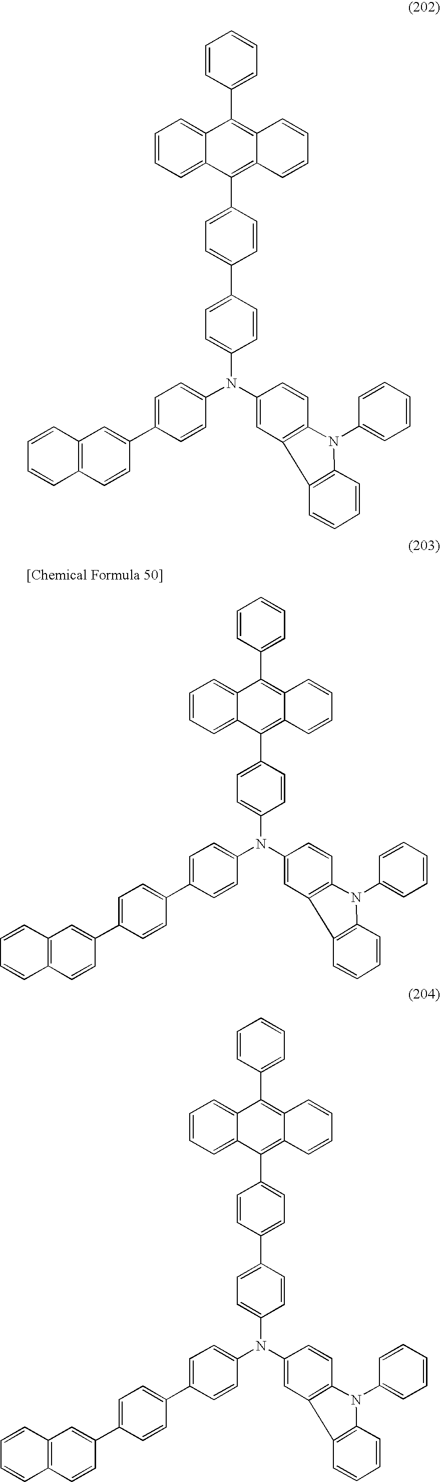

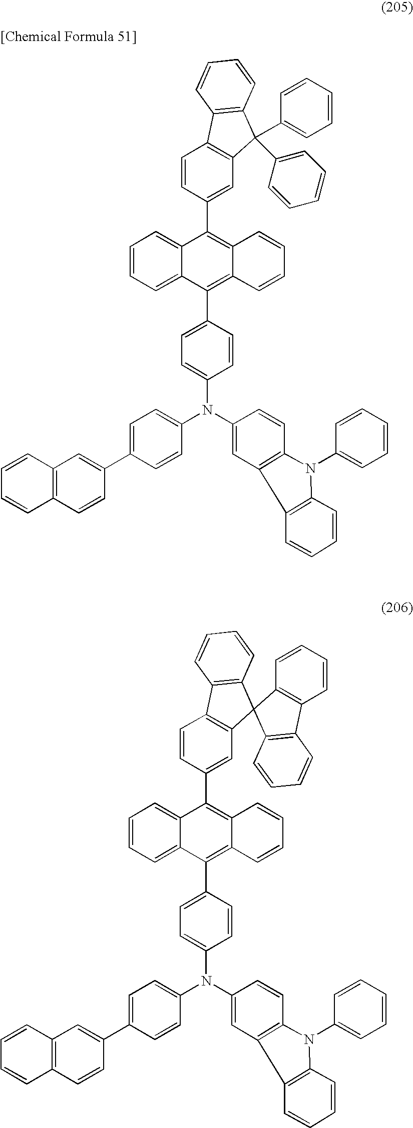

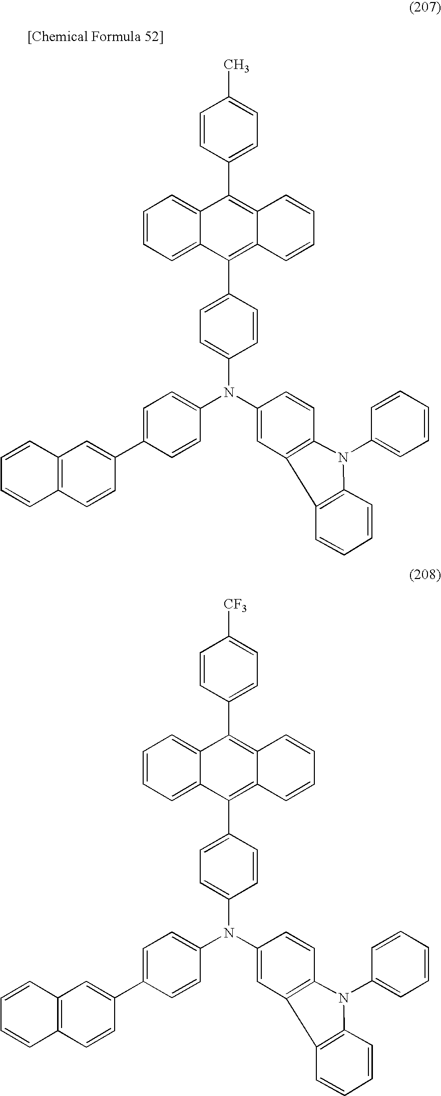

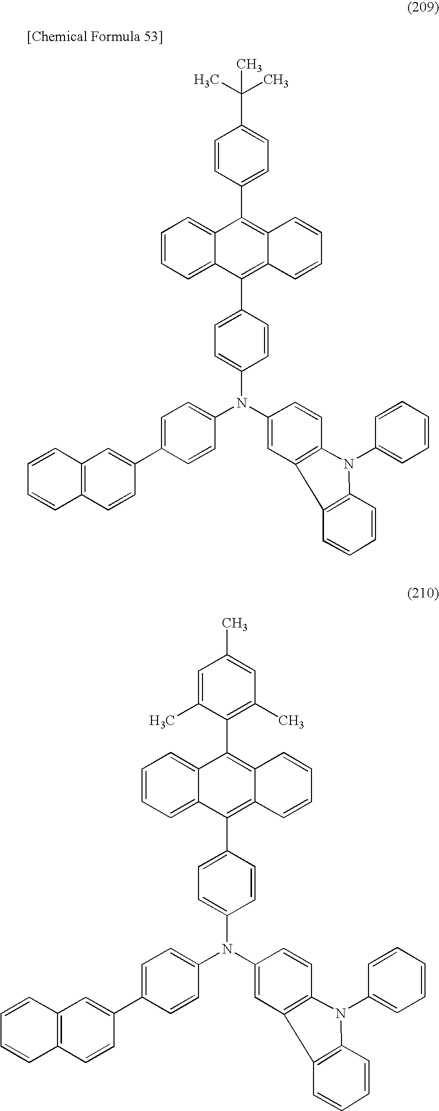

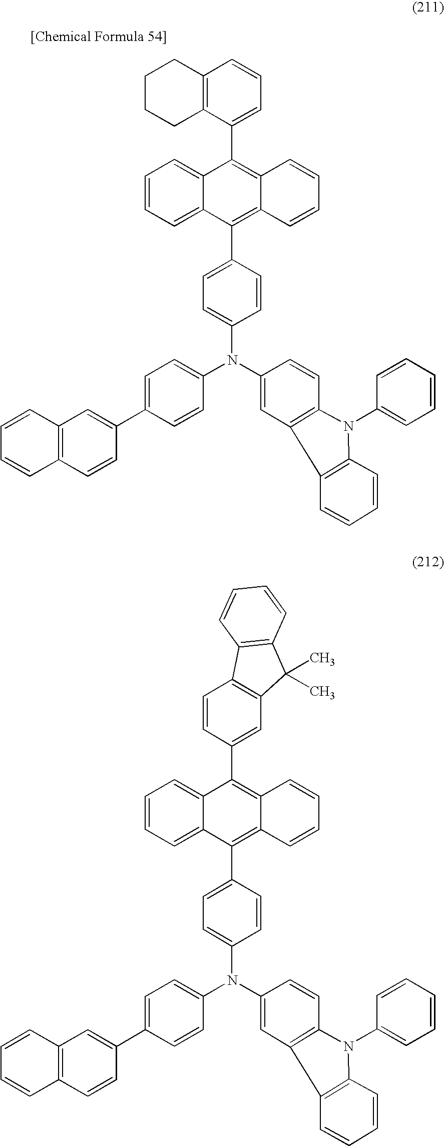

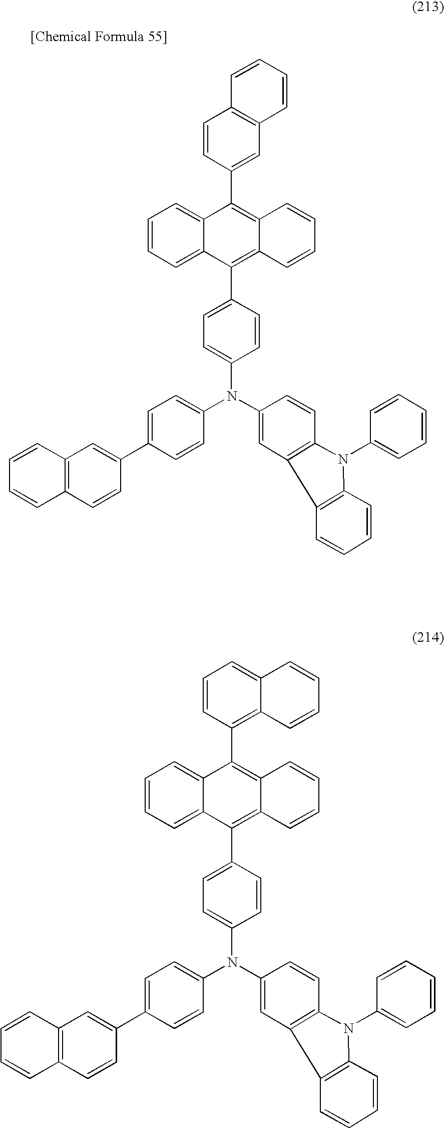

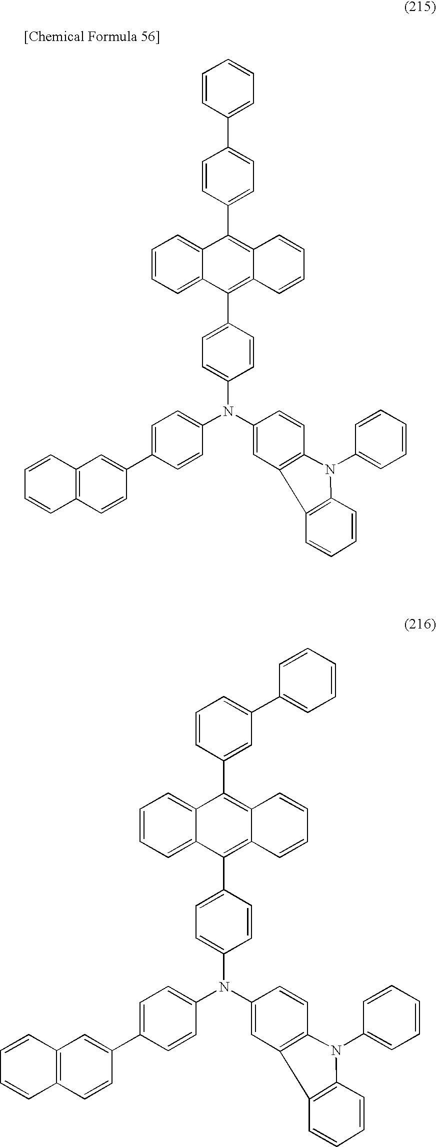

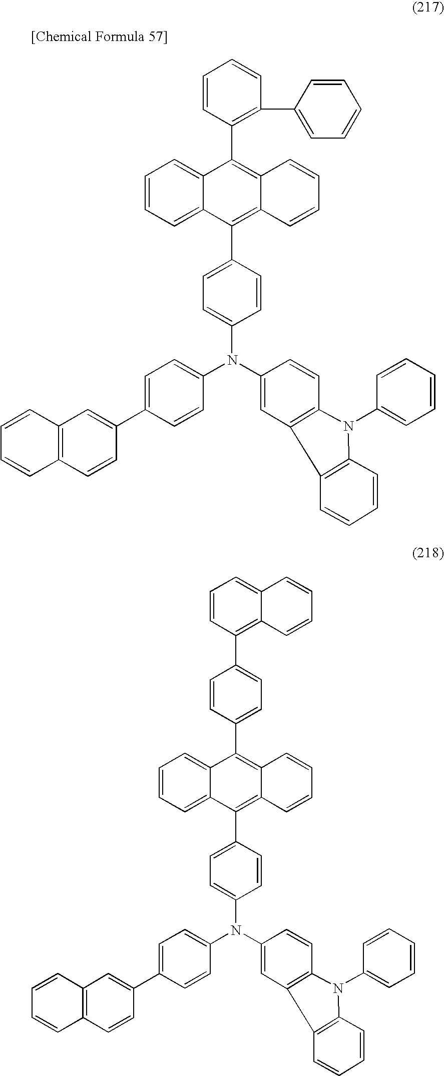

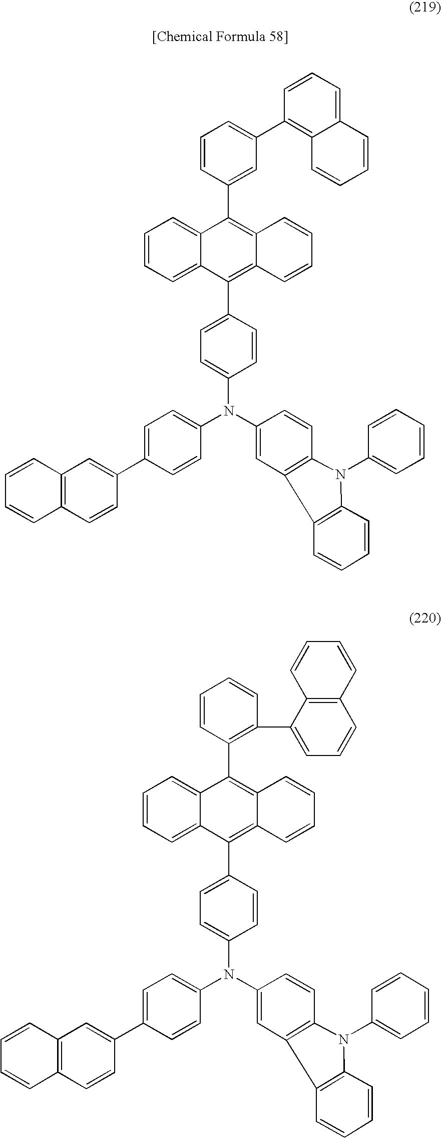

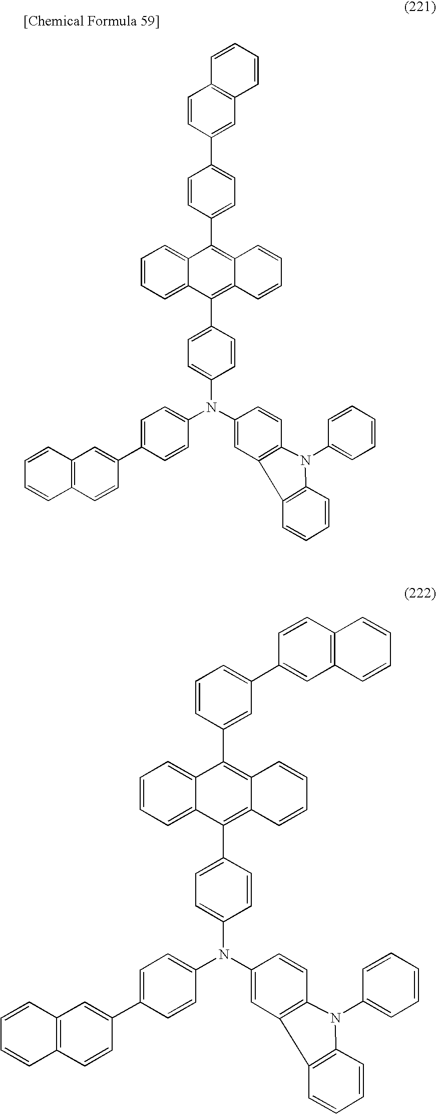

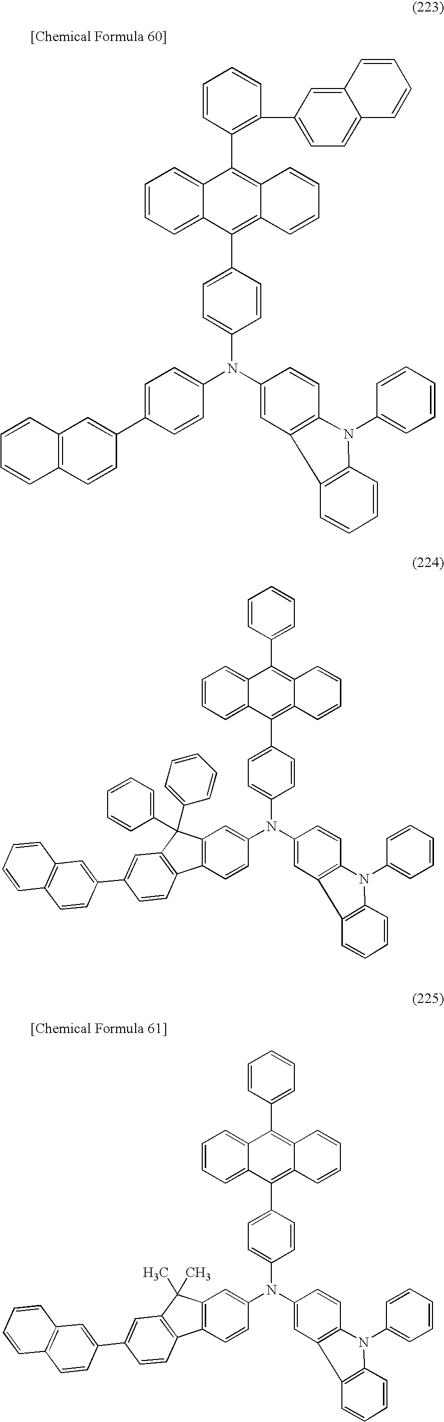

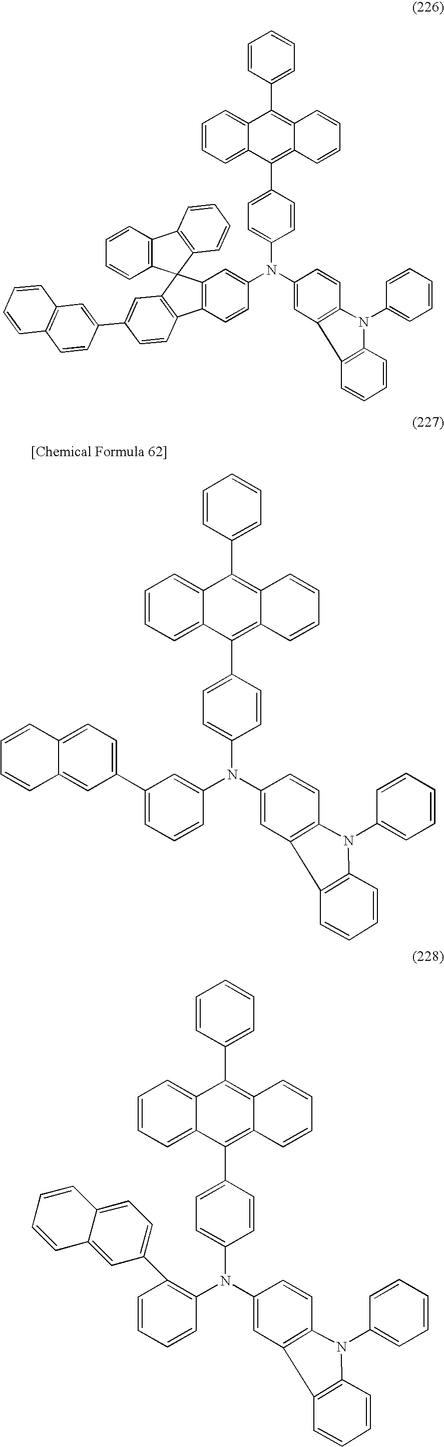

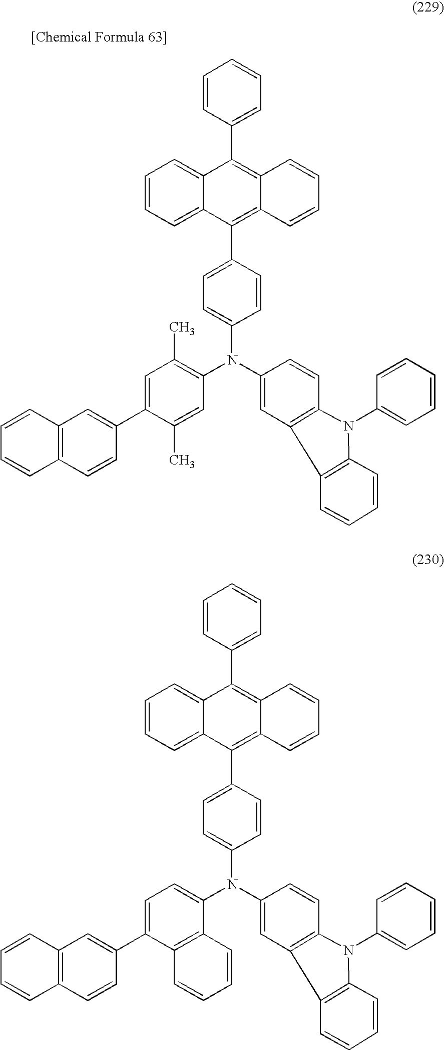

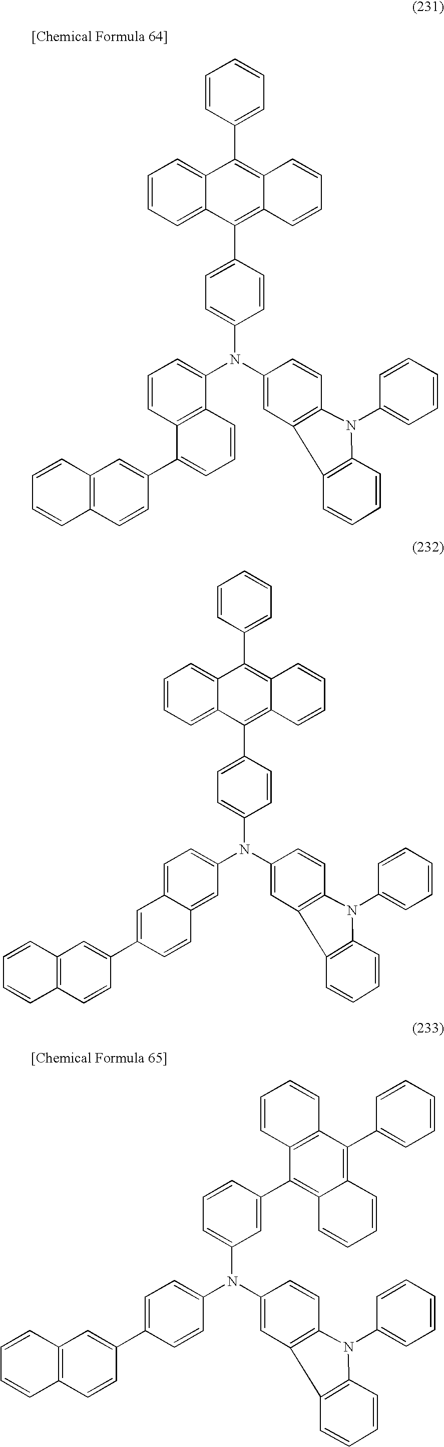

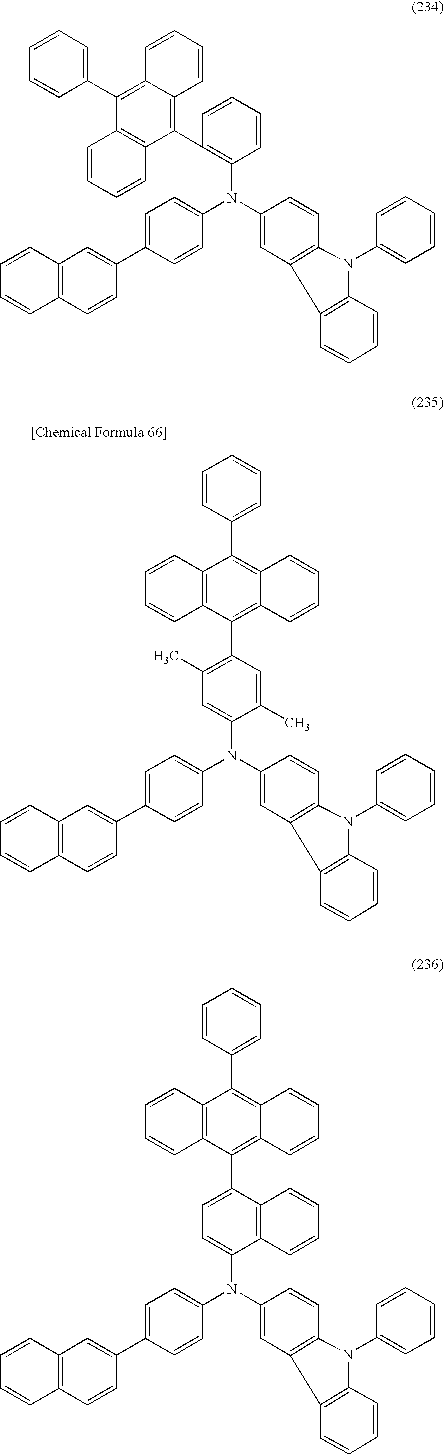

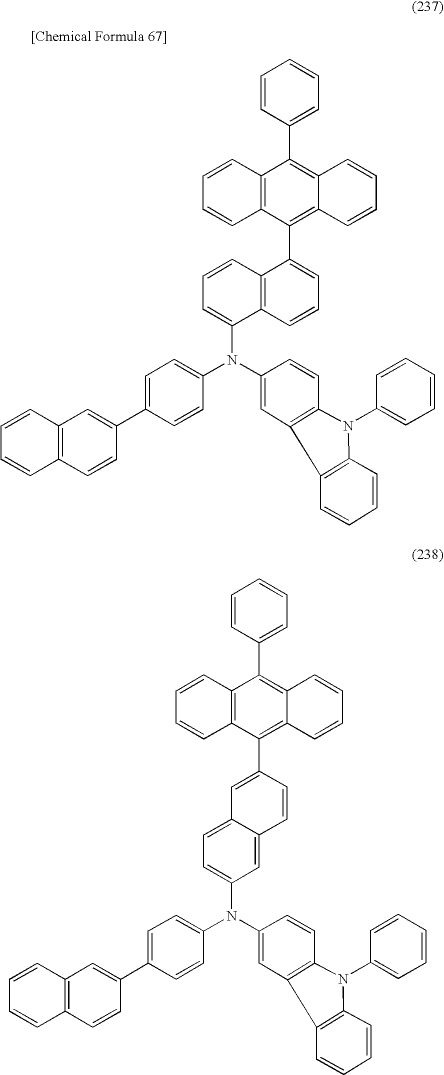

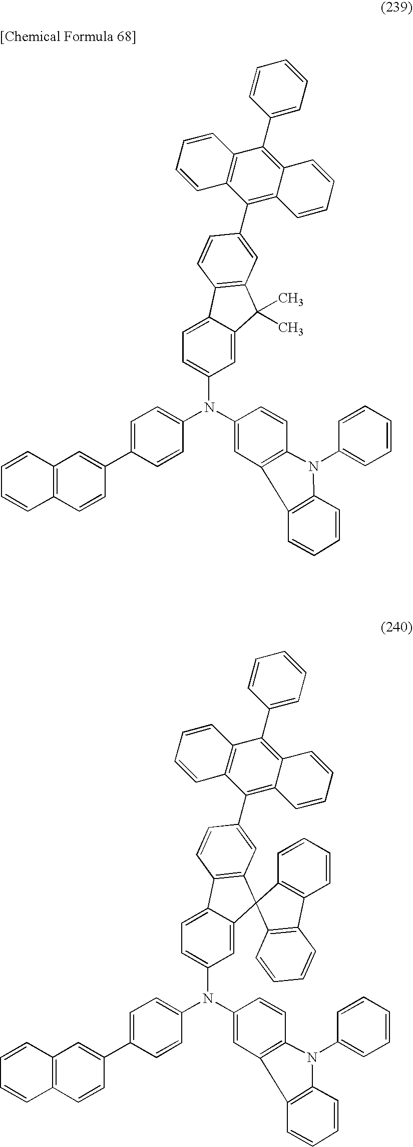

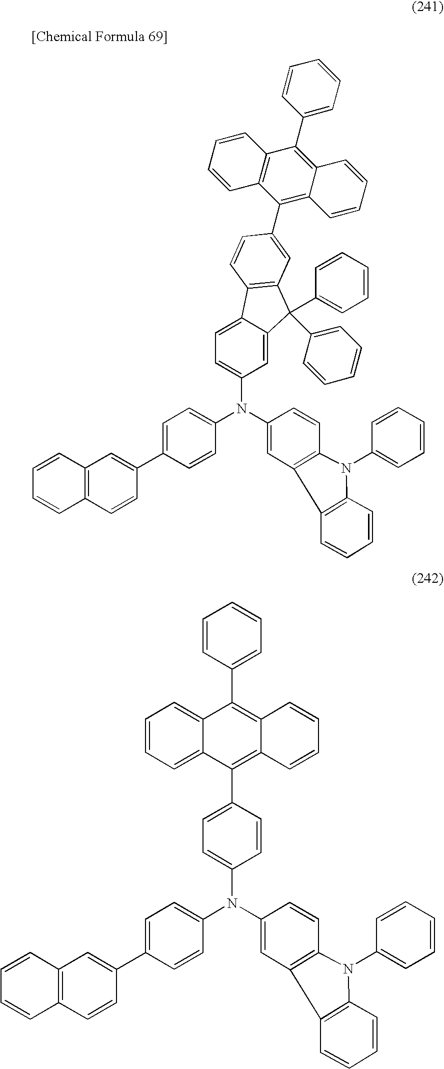

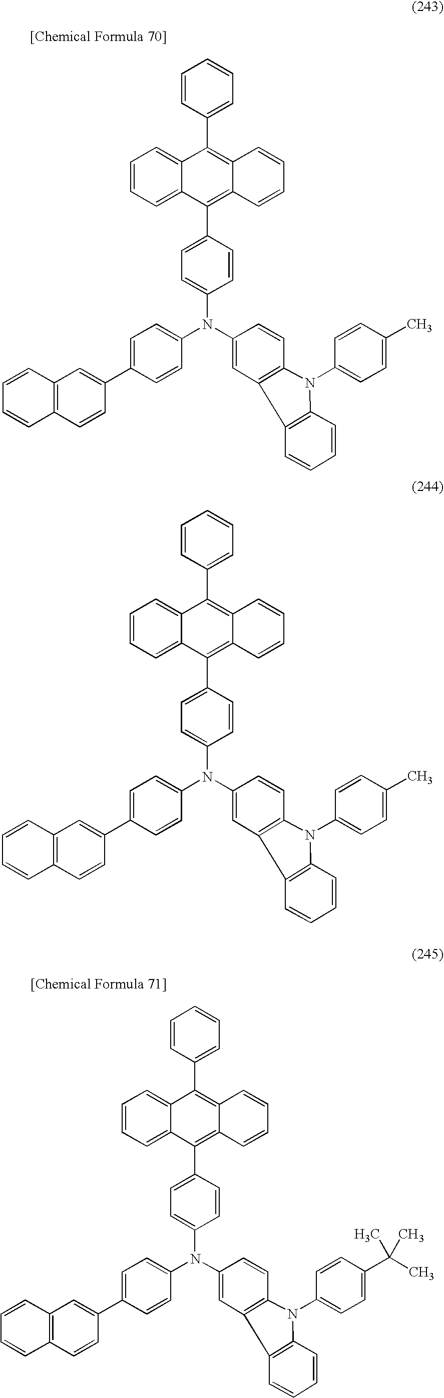

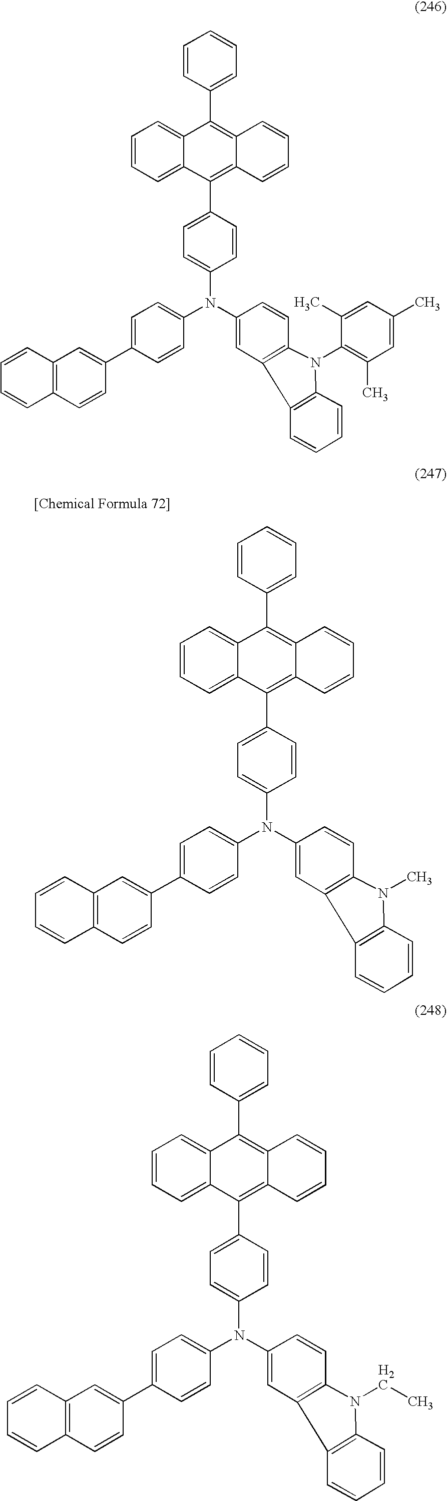

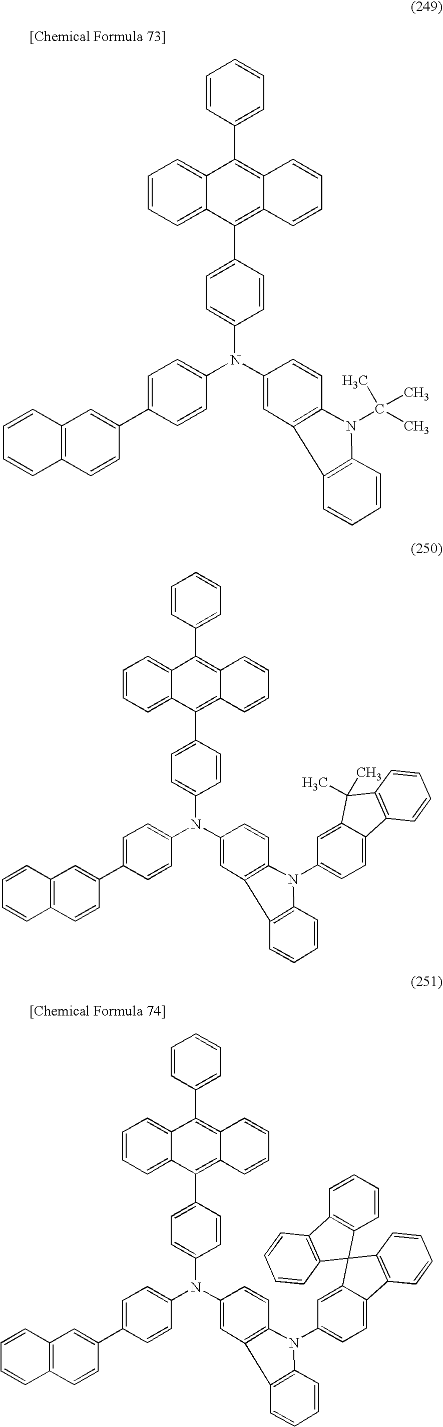

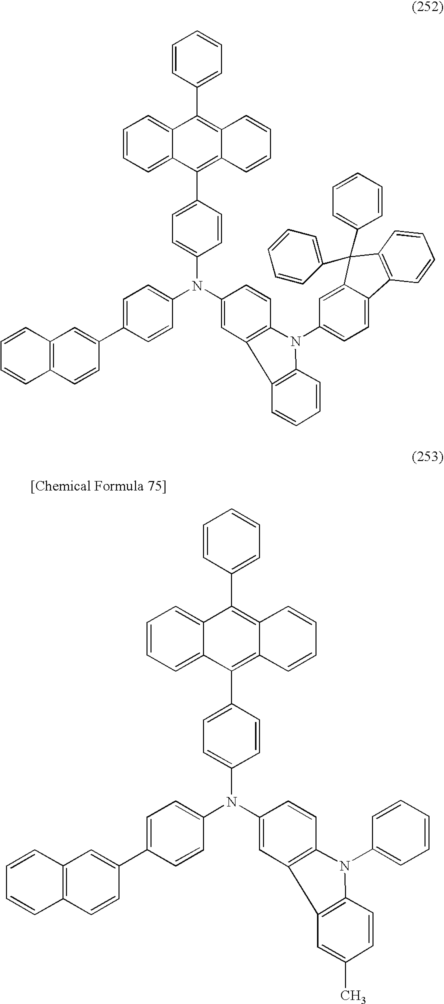

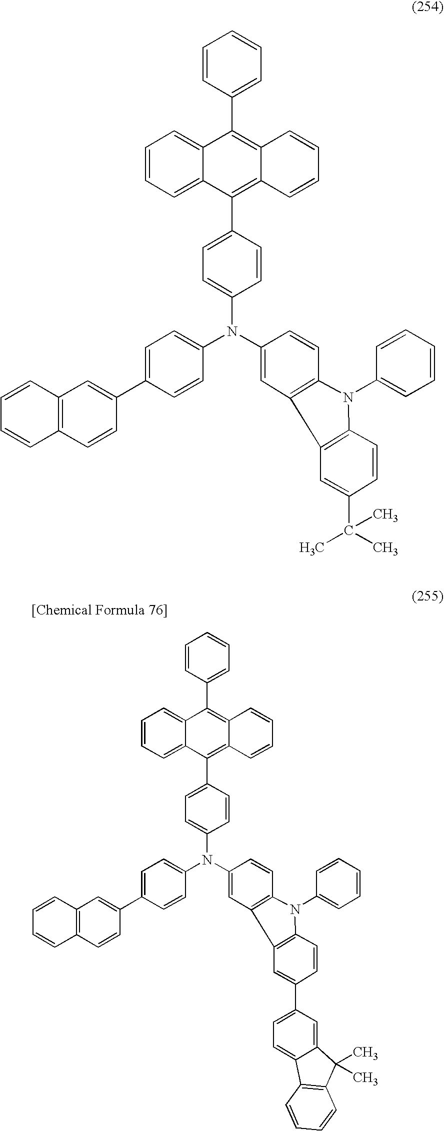

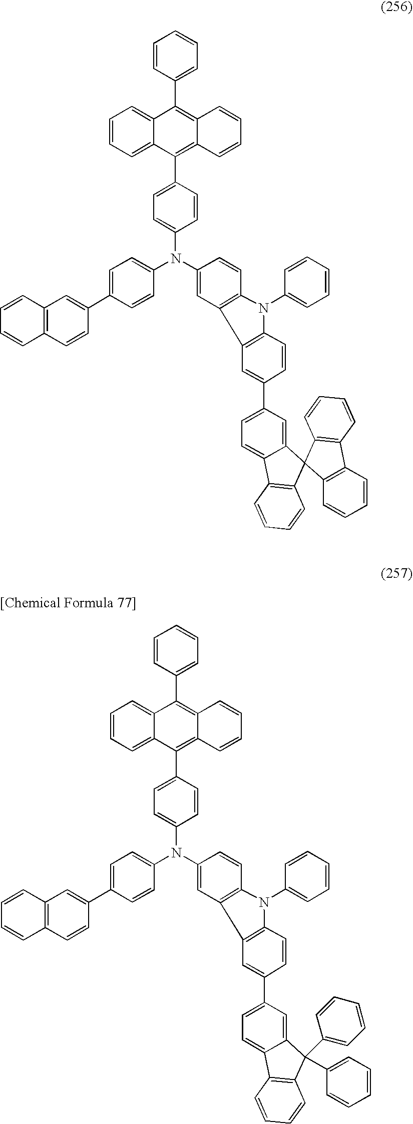

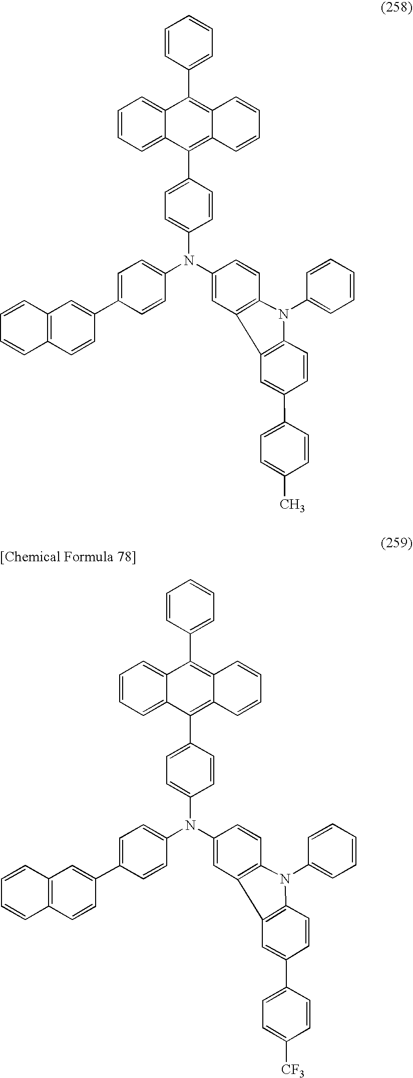

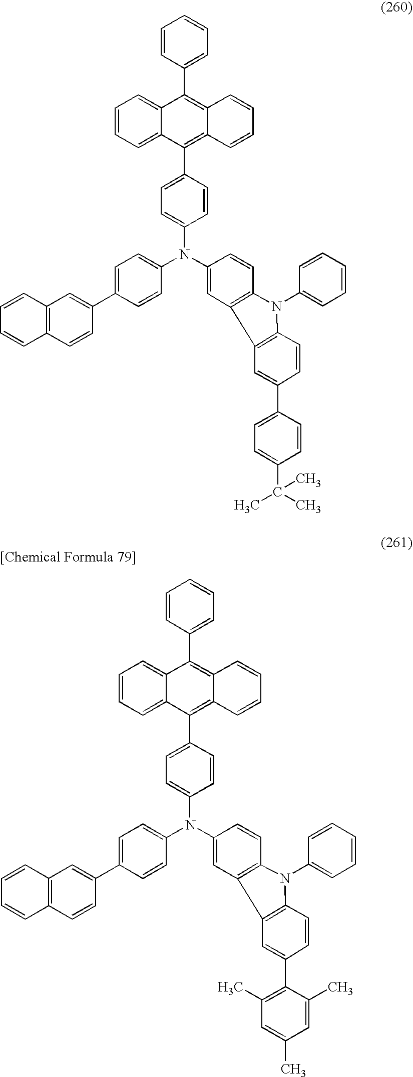

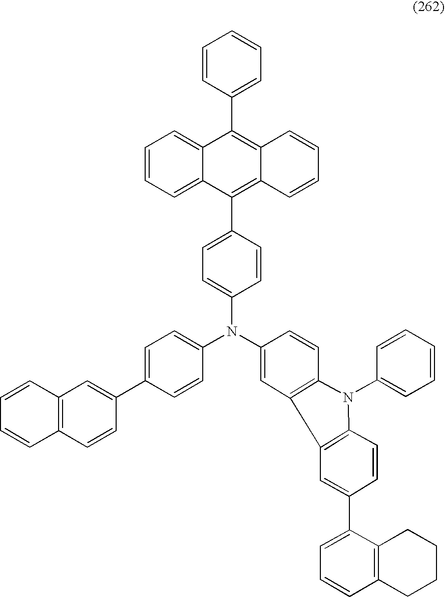

Furthermore, since the organic compounds used for the synthesis of the anthracene derivatives described in this embodiment are also novel materials, the organic compounds used for the synthesis of the anthracene derivatives of this embodiment are also included in an embodiment the present invention. Thus, an embodiment of the present invention is an organic compound represented by General Formula (G2) below.

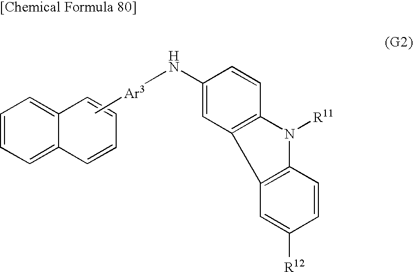

In General Formula (G2) above, Ar³ represents a substituted or unsubstituted arylene group having 6 to 13 carbon atoms. When Ar³ has substituents, the substituents may be bonded to each other to form a ring. Furthermore, when a carbon atom of Ar³ has two substituents, the substituents may be bonded to each other to form a spiro ring. In General Formula (G2) above, R¹¹ represents either an alkyl group having 1 to 4 carbon atoms or a substituted or unsubstituted aryl group having 6 to 13 carbon atoms. When R¹¹ is an aryl group having substituents, the substituents may be bonded to each other to form a ring. Furthermore, when a carbon atom of R¹¹ has two substituents, the substituents may be bonded to each other to form a spiro ring. In General Formula (G2) above, R¹² represents hydrogen, an alkyl group having 1 to 4 carbon atoms, or a substituted or unsubstituted aryl group having 6 to 13 carbon atoms. When R¹² is an aryl group having substituents, the substituents may be bonded to each other to form a ring. Furthermore, when a carbon atom of R¹² has two substituents, the substituents may be bonded to each other to form a spiro ring.

In General Formula (G2) above, structures represented by Structural Formulae (5-1) to (5-11) can be given as an example of arylene group represented by Ar³.

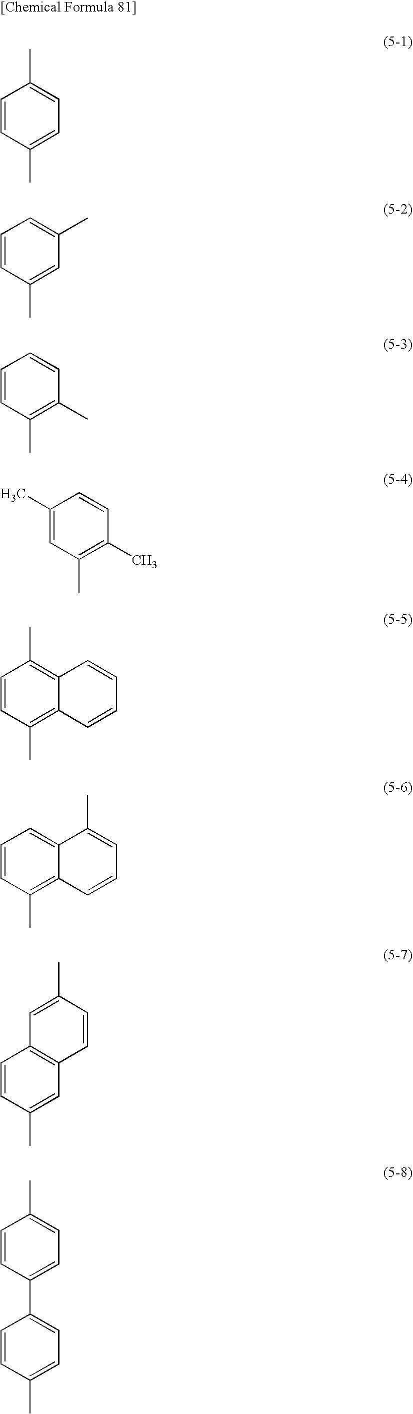

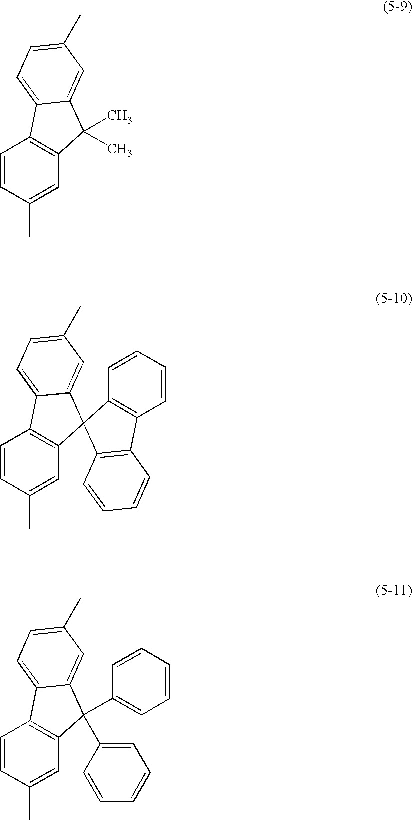

Further, in General Formula (G2) above, structures represented by Structural Formulae (6-1) to (6-28) can be given as an example of an arylene group represented by R¹¹.

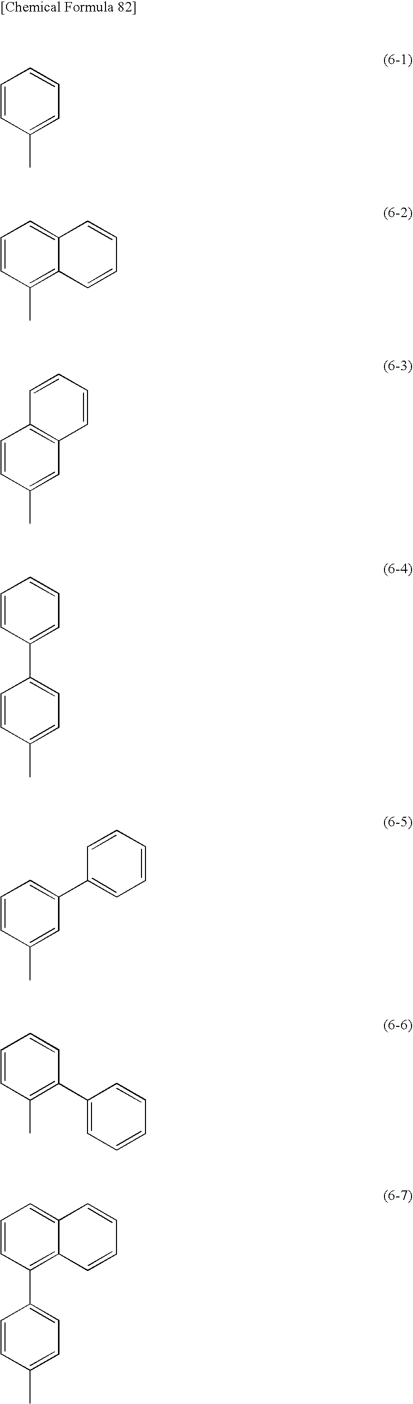

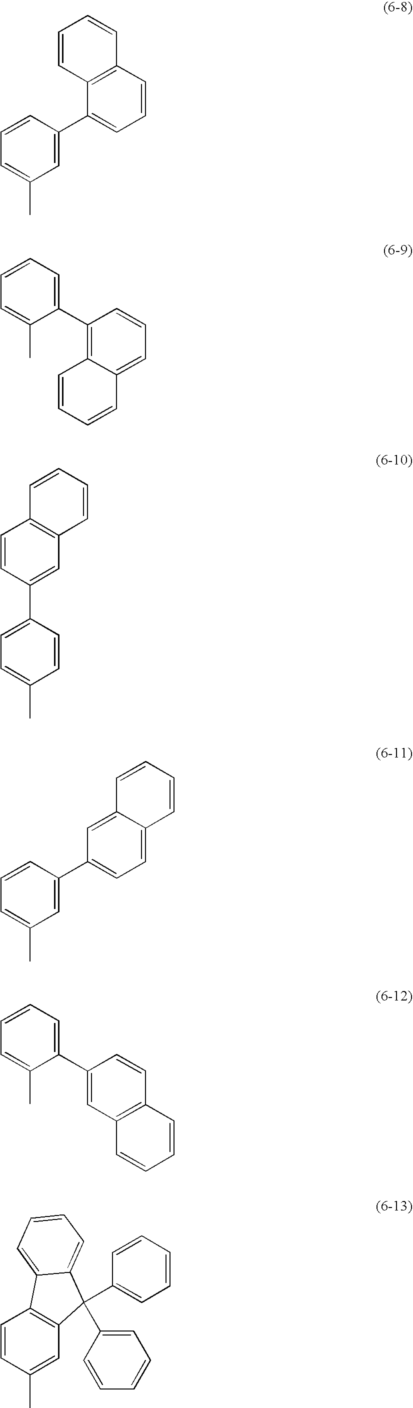

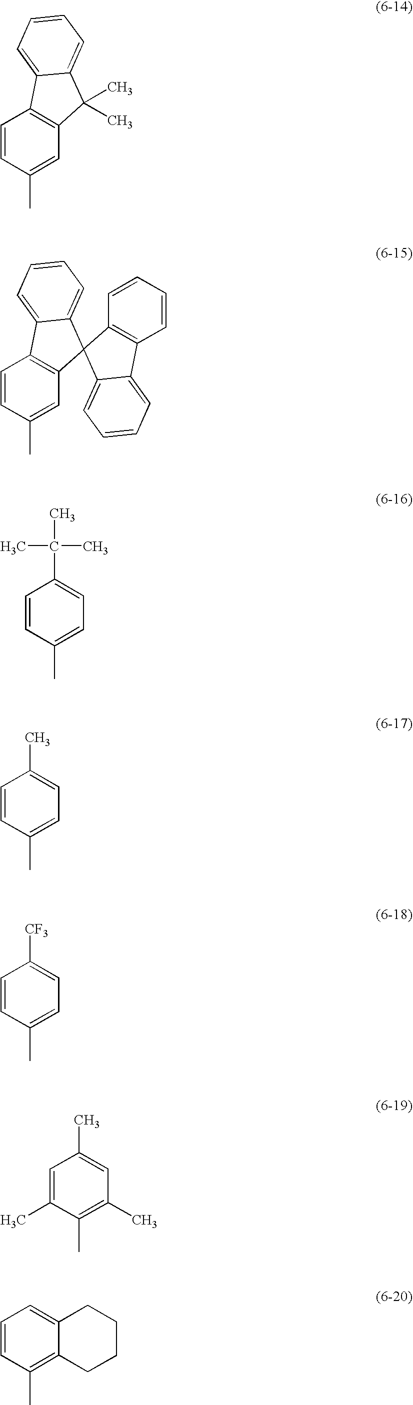

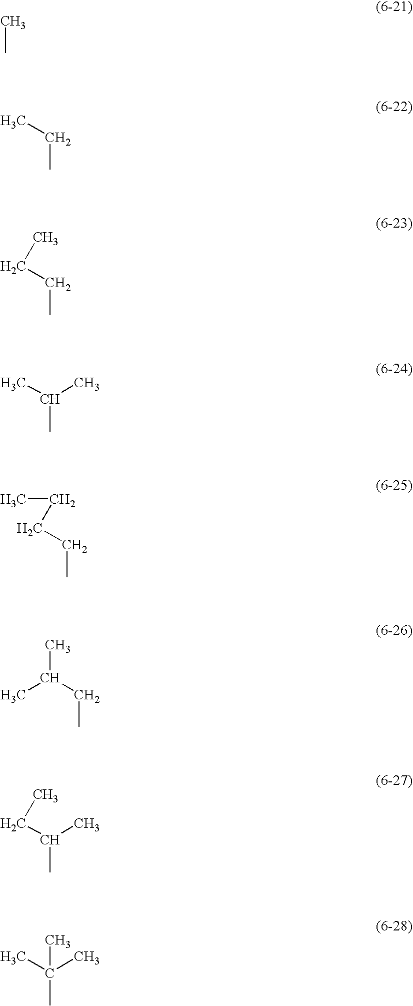

Further, in General Formula (G2) above, structures represented by Structural Formulae (7-0) to (7-28) can be given as an example of an arylene group represented by R¹².

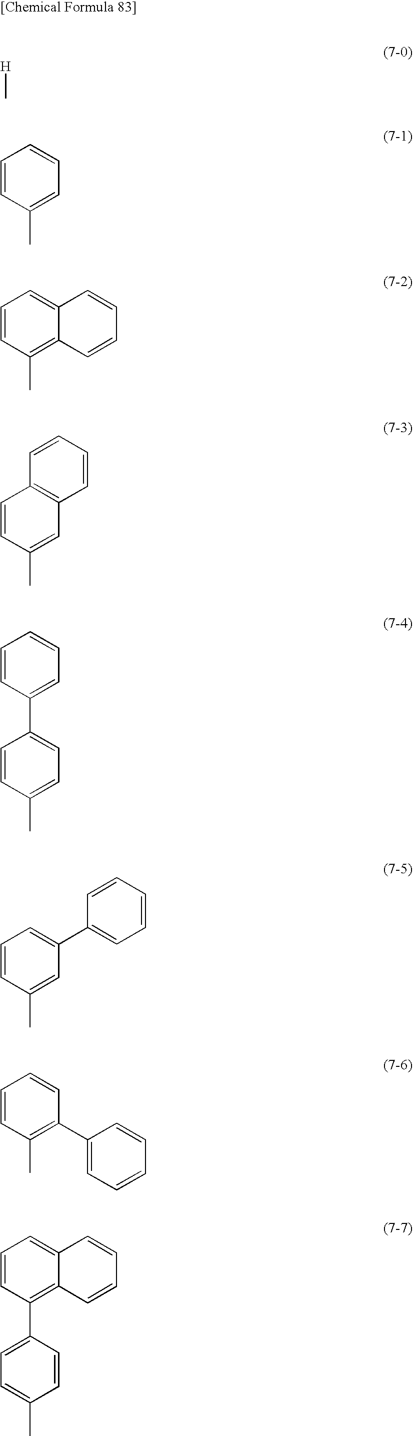

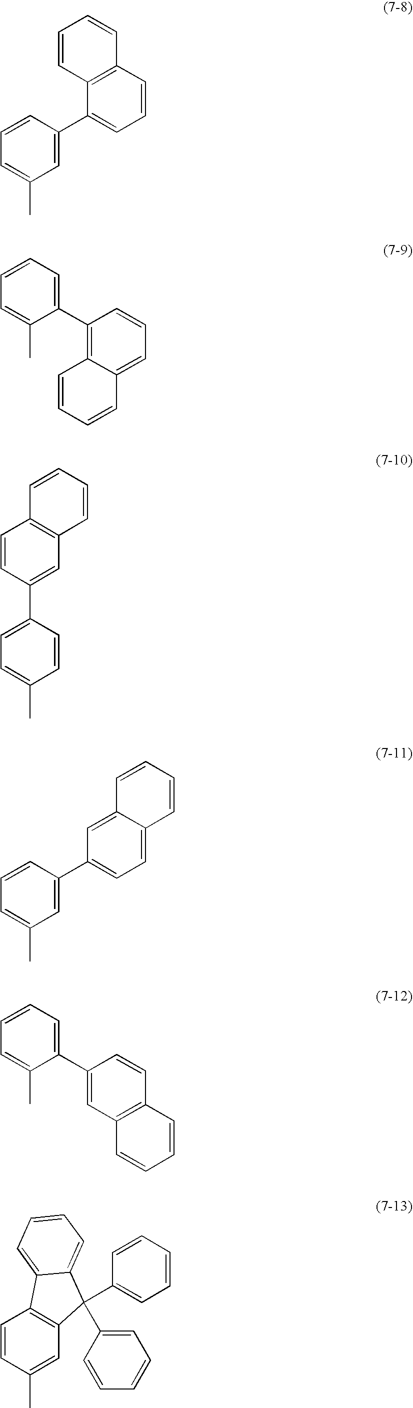

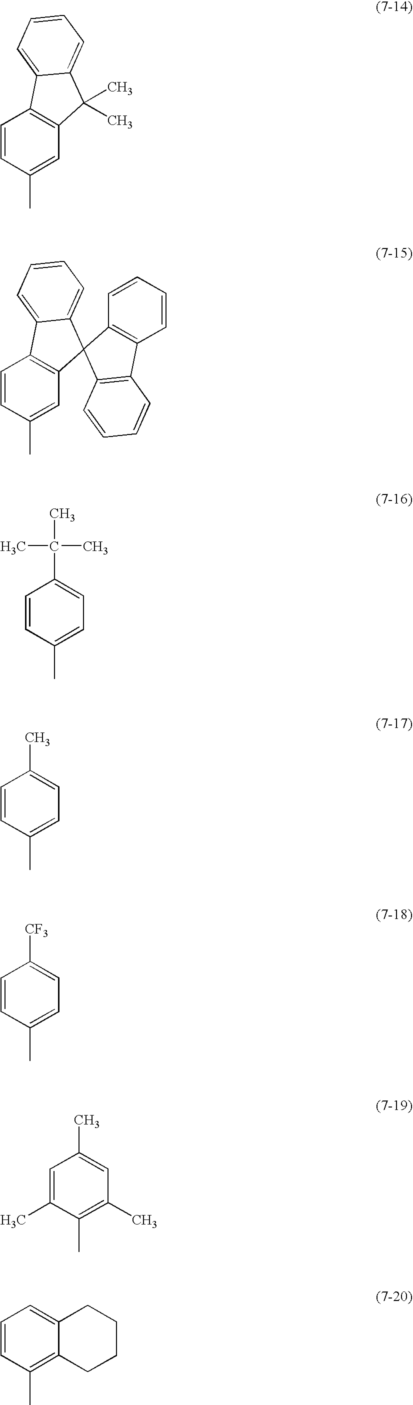

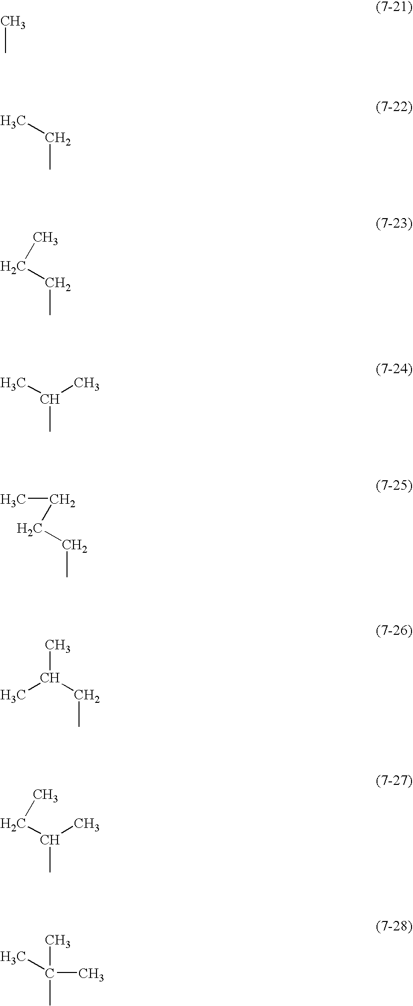

Specific examples of such organic compounds used in the synthesis of the anthracene derivatives of this embodiment can be anthracene derivatives represented by Structural Formulae (301) to (333) and by Structural Formulae (401) to (433) below. However, the present invention is not limited to these examples.

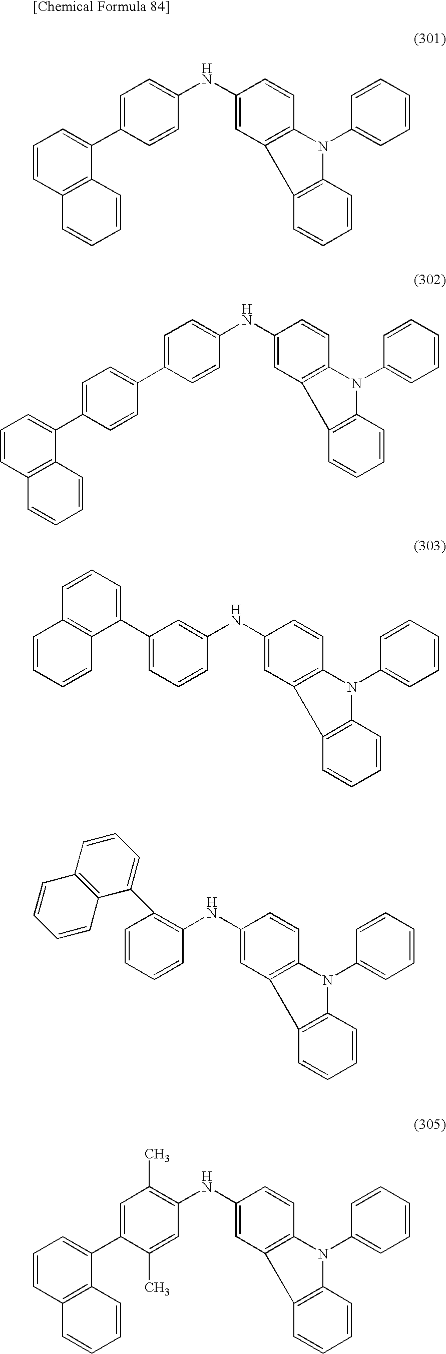

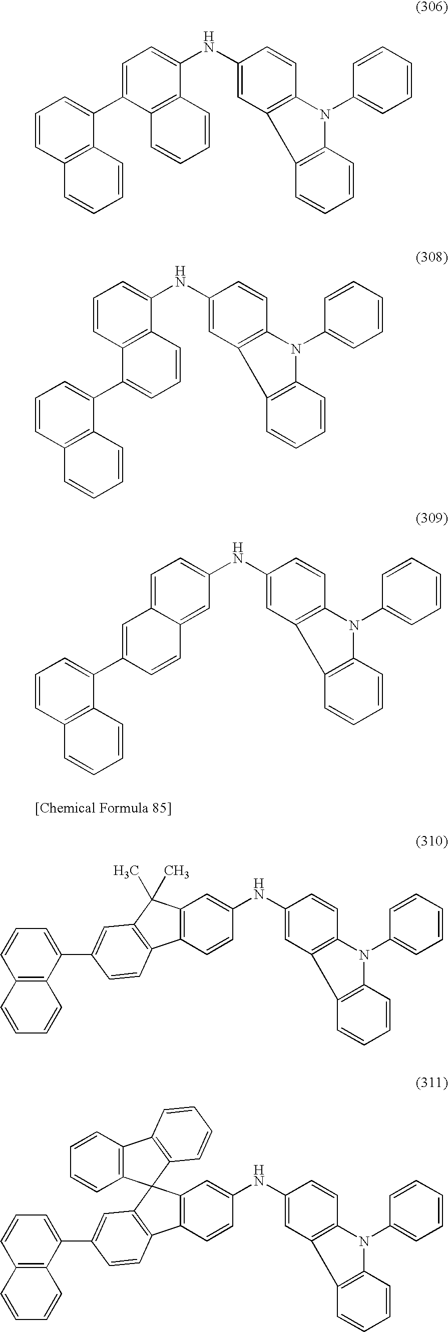

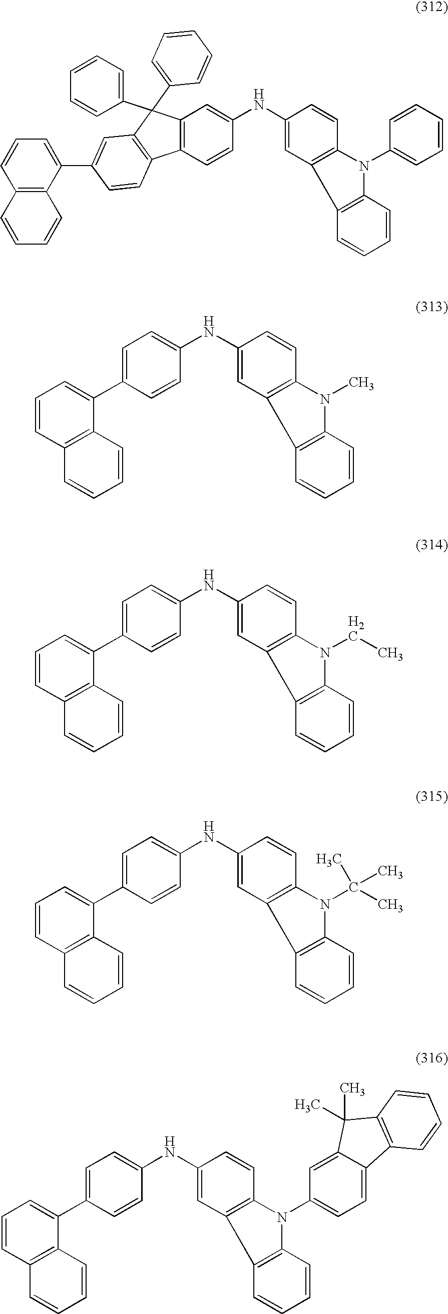

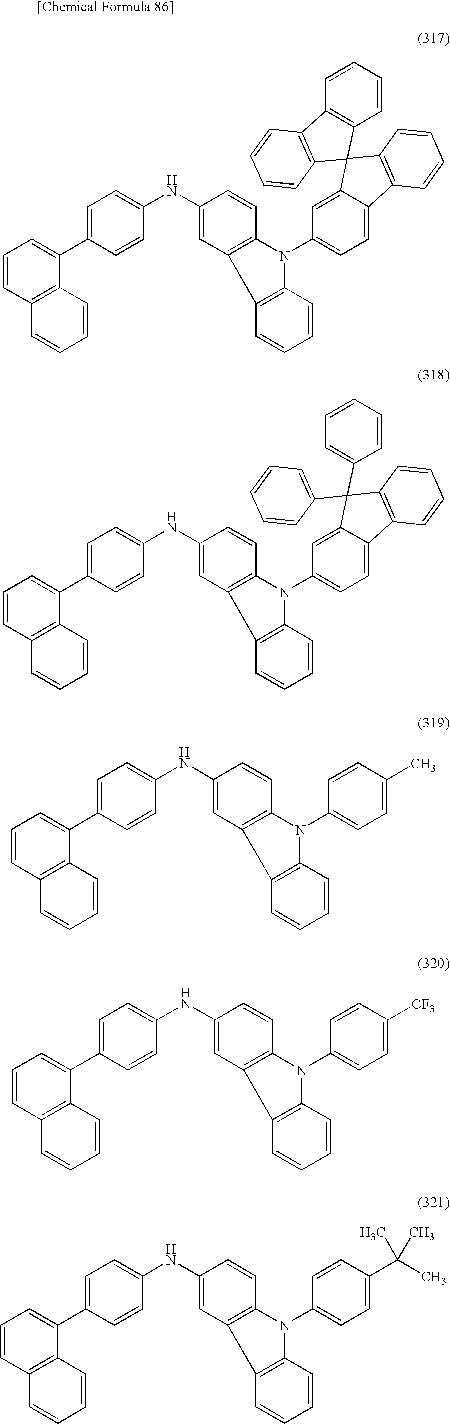

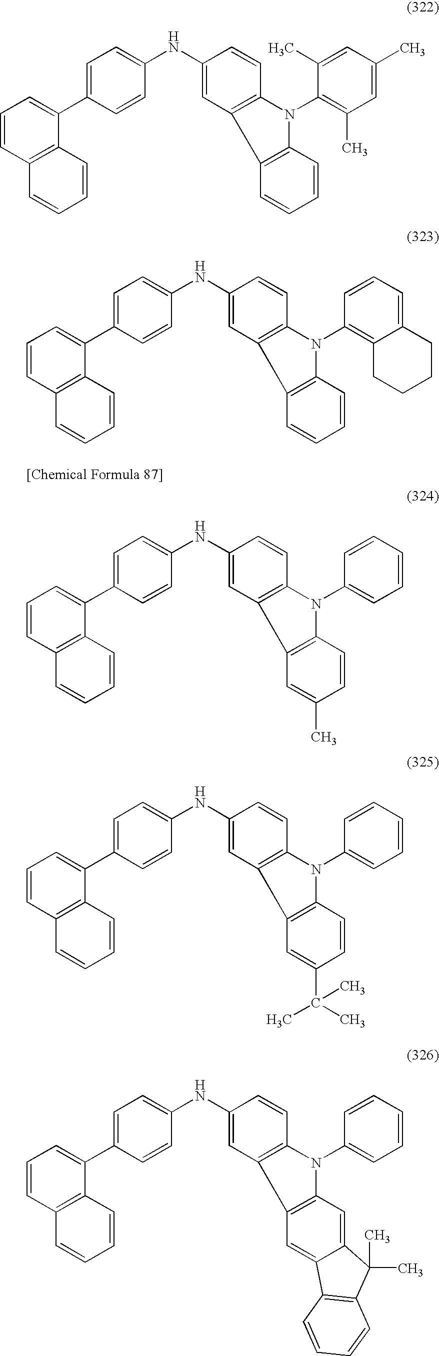

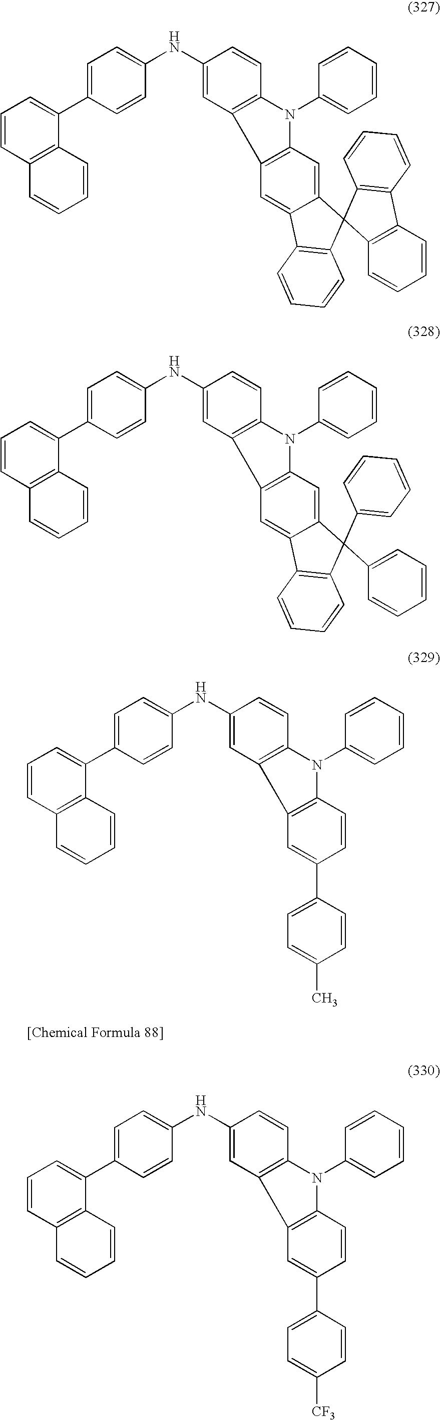

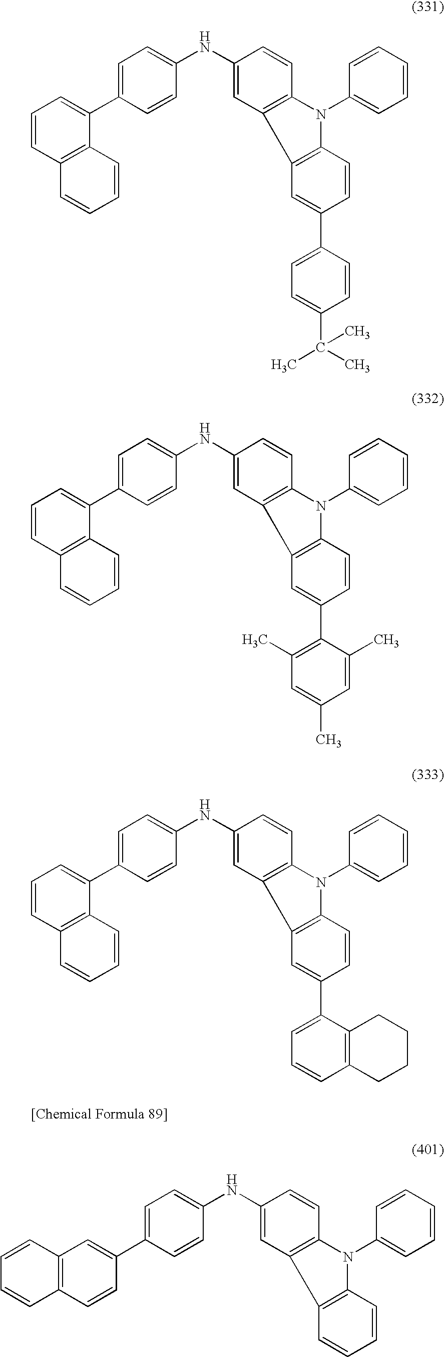

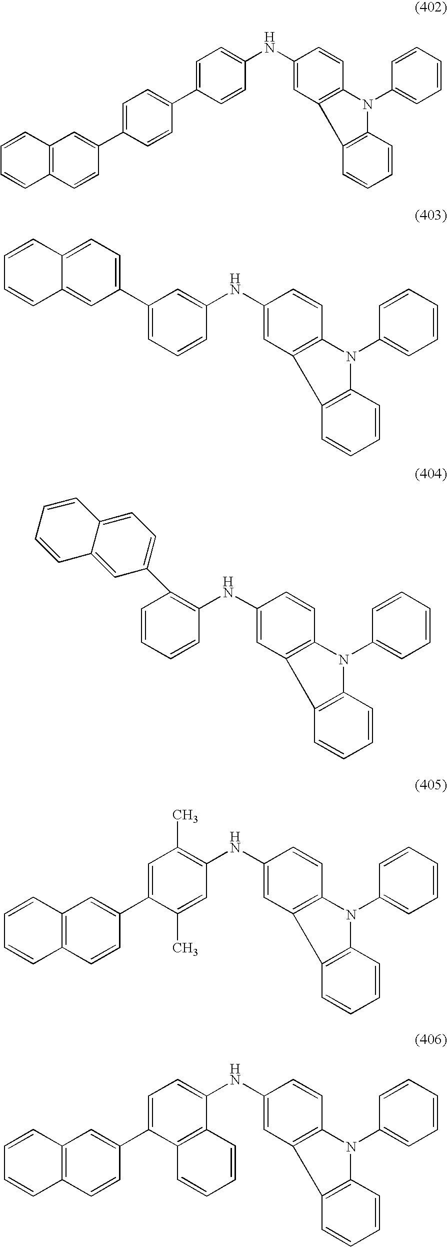

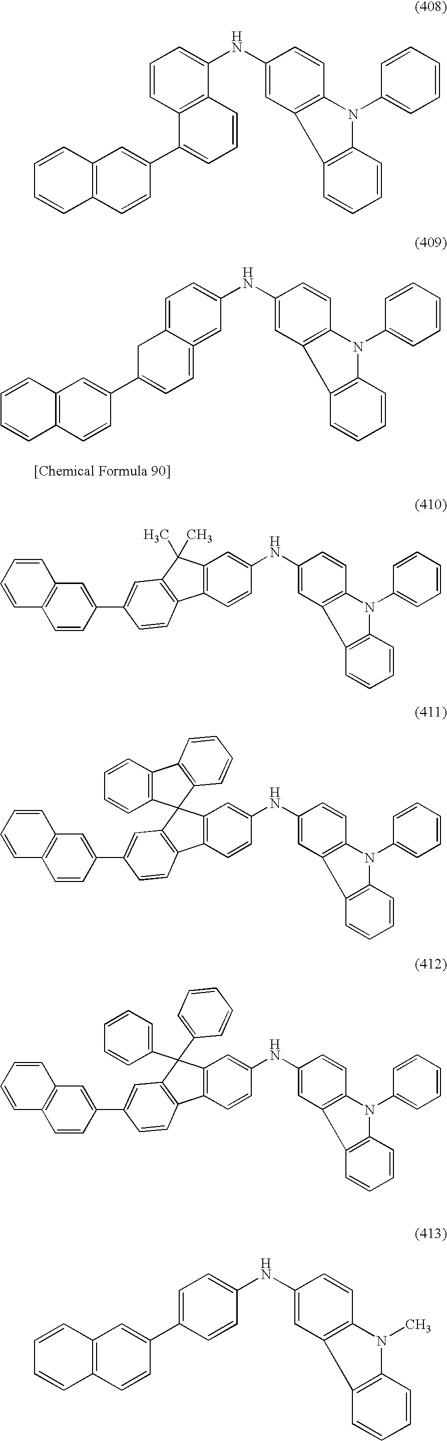

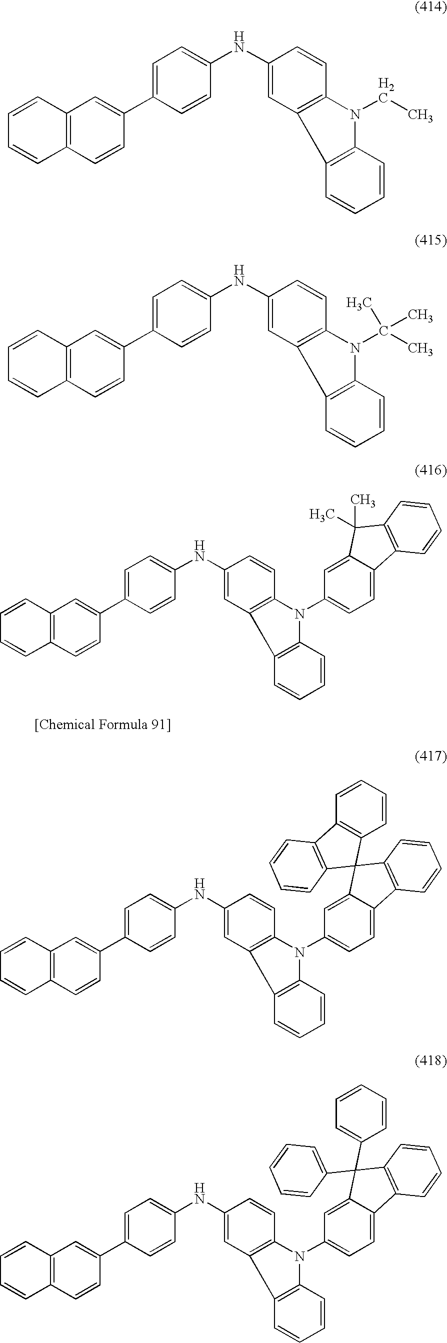

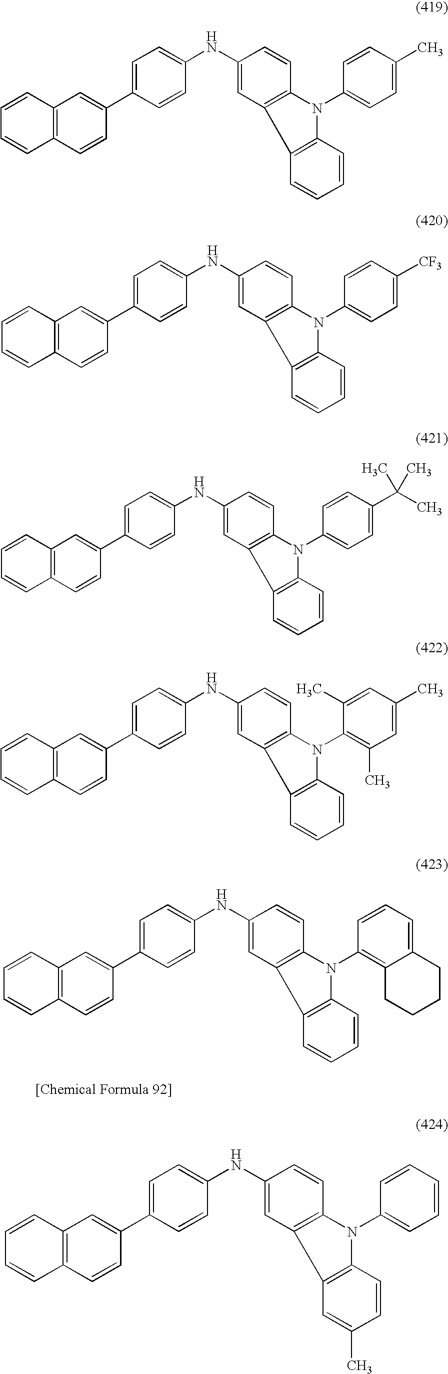

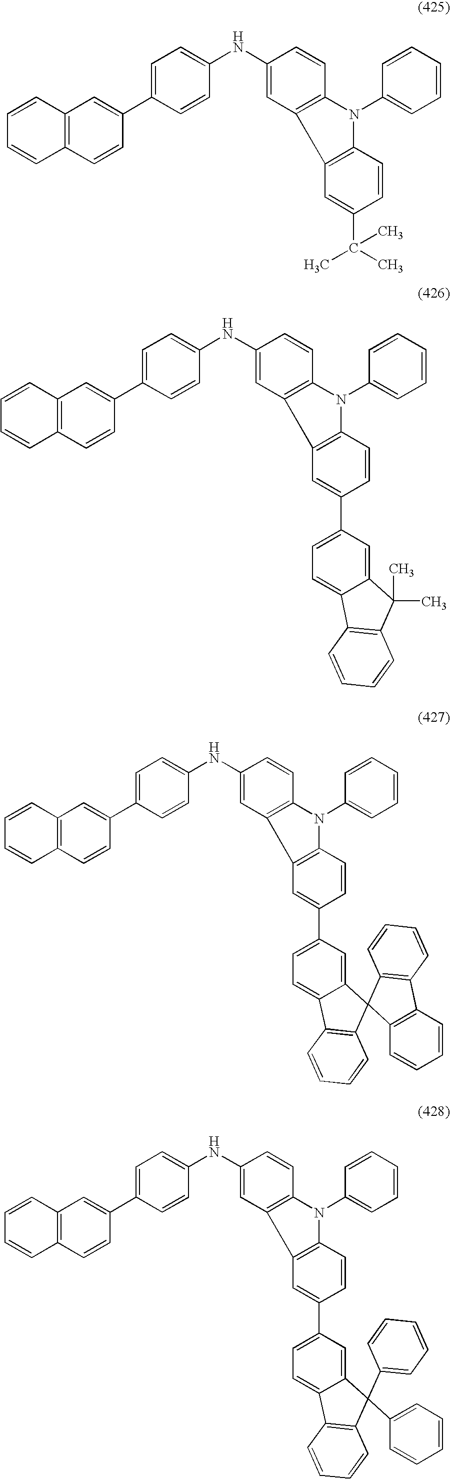

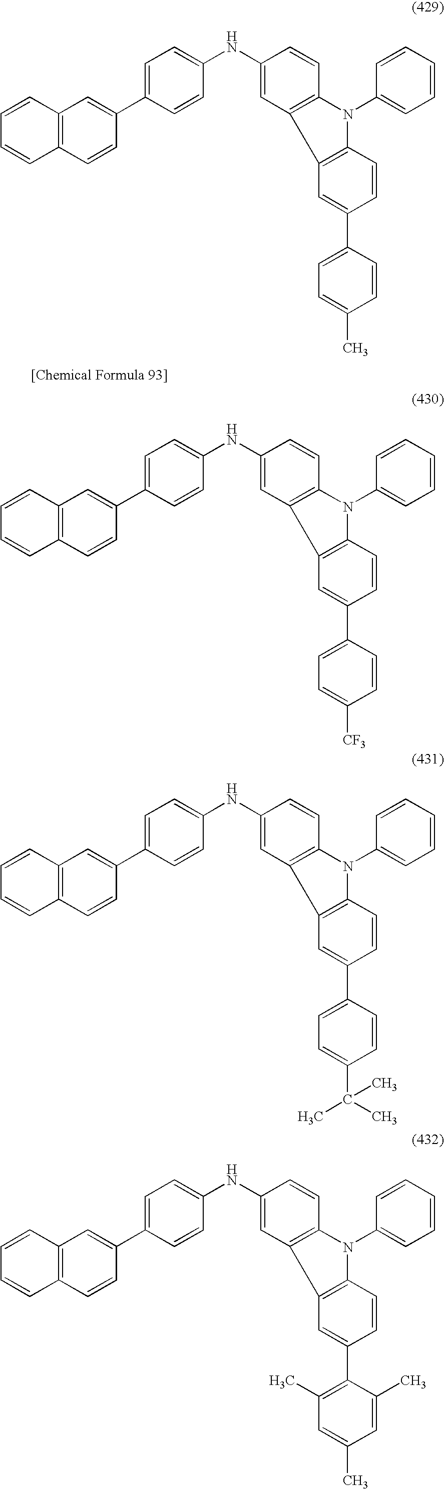

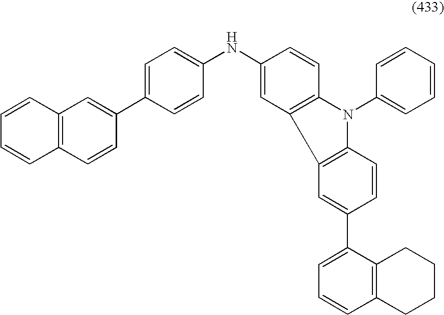

A variety of reactions can be applied to a synthetic method of the anthracene derivatives of this embodiment and a synthetic method of organic compounds used for the synthesis of the anthracene derivatives. For example, the synthesis can be performed by applying synthetic methods illustrated in Reaction Schemes 1 to 6 below. Since the organic compounds used for the synthesis of the anthracene derivatives are novel materials, they are included in an embodiment of the present invention.

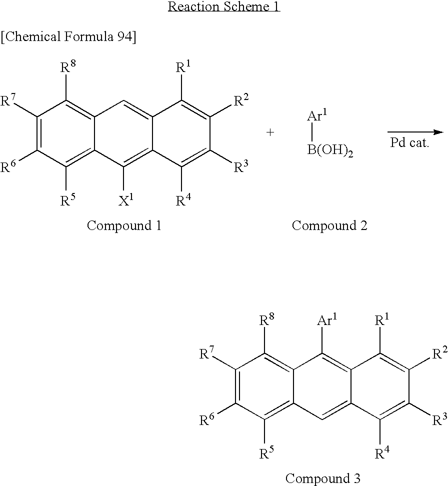

First, an anthracene derivative (Compound 1) and arylboronic acid (Compound 2) are subjected to the coupling by the Suzuki-Miyaura reaction using a palladium catalyst, whereby 9-arylanthracene derivative (Compound 3) can be obtained (Reaction Scheme 1).

In Reaction Scheme 1, X¹ represents a halogen or a triflate group, and iodine, bromine, and chlorine are preferred as the halogen. In Reaction Scheme 1, R¹ to R⁸ represent either hydrogen or an alkyl group having 1 to 4 carbon atoms. In Reaction Scheme 1, Ar¹ represents a substituted or unsubstituted aryl group having 6 to 13 carbon atoms. When Ar¹ has substituents, the substituents may be bonded to each other to form a ring. Furthermore, when a carbon atom of Ar¹ has two substituents, the substituents may be bonded to each other to form a spiro ring. In this reaction, an arylorganoboron compound which is obtained by protecting the boronic acid of Compound 2 using ethylene glycol, pinacol, or the like may be used instead of Compound 2.

Examples of palladium catalysts which can be used in Reaction Scheme 1 include palladium(II)acetate, tetrakis(triphenylphosphine)palladium(0), and the like. Examples of ligands of palladium catalysts which can be used in Reaction Scheme 1 include tri(o-tolyl)phosphine, triphenylphosphine, tricyclohexylphosphine, and the like. Examples of bases which can be used in Reaction Scheme 1 include organic bases such as sodium tert-butoxide, inorganic bases such as potassium carbonate, and the like. Examples of solvents which can be used in Reaction Scheme 1 include a mixed solvent of toluene and water; a mixed solvent of toluene, alcohol such as ethanol, and water; a mixed solvent of xylene and water; a mixed solvent of xylene, alcohol such as ethanol, and water; a mixed solvent of benzene and water; a mixed solvent of benzene, alcohol such as ethanol, and water; a mixed solvent of ether such as ethylene glycol dimethyl ether, and water; and the like. Use of a mixed solvent of toluene and water or a mixed solvent of toluene, ethanol, and water is more preferred.

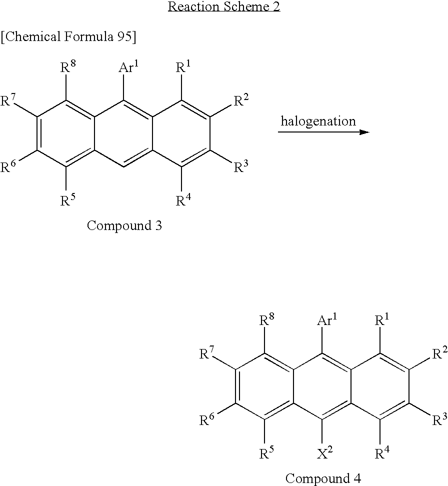

Next, a halogenated arylanthracene derivative (Compound 4) can be obtained by the halogenation of 9-arylanthracene derivative (Compound 3) obtained by Reaction Scheme 1 (Reaction Scheme 2).

In Reaction Scheme 2, X² represents a halogen, and iodine, bromine, and chlorine are preferred as the halogen. In Reaction Scheme 2, to R⁸ each represent either hydrogen or an alkyl group having 1 to 4 carbon atoms. In Reaction Scheme 2, Ar¹ represents a substituted or unsubstituted aryl group having 6 to 13 carbon atoms. When Ar¹ has substituents, the substituents may be bonded to each other to form a ring. Furthermore, when a carbon atom of Ar¹ has two substituents, the substituents may be bonded to each other to form a spiro ring.

In the case where bromination is carried out in Reaction Scheme 2, bromine, N-bromosuccinimide, and the like can be given as an example of a bromination agent. Examples of solvents which can be used in bromination with bromine include halogen-based solvents such as chloroform and carbon tetrachloride. Examples of solvents which can be used in bromination with N-bromosuccinimide are ethyl acetate, tetrahydrofuran, dimethylformamide, acetic acid, water, and the like.

In the case where iodination is carried out in Reaction Scheme 2, N-iodosuccinimide, 1,3-diiodo-5,5-dimethylimidazolidine-2,4-dione (abbreviated to DIH), 2,4,6,8-tetraiodo-2,4,6,8-tetraazabicyclo[3,3,0]octane-3,7-dione, 2-iodo-2,4,6,8-tetraazabicyclo[3,3,0]octane-3,7-dione, and the like can be used as an iodination reagent. When the iodination is carried out by using these iodination agents, the following can be used alone or in combination: an aromatic hydrocarbon such as benzene, toluene, or xylene; an ether such as 1,2-dimethoxyethane, diethyl ether, methyl-t-butyl ether, tetrahydrofuran, or dioxane; a saturated hydrocarbon such as pentane, hexane, heptane, octane, or cyclohexane; a halogen such as dichloromethane, chloroform, tetrachloromethane, 1,2-dichloroethane, or 1,1,1-trichloroethane; a nitrile such as acetonitrile or benzonitrile; an ester such as ethyl acetate, methyl acetate, or butyl acetate; acetic acid (glacial acetic acid); water; or the like. When water is used, it is preferred to be mixed with an organic solvent. Moreover, it is preferred that the reaction be conducted in the presence of an acid such as sulfuric acid and acetic acid simultaneously.

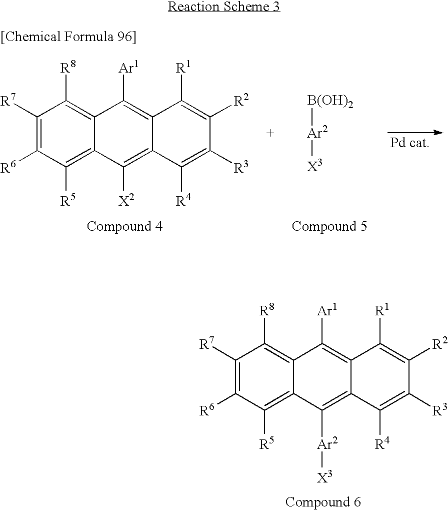

Next, the arylanthracene derivative (Compound 4) obtained by Reaction Scheme 2 and a boronic acid which is a halogenated aryl derivative (Compound 5) are subjected to the coupling by the Suzuki-Miyaura reaction using a palladium catalyst, whereby a halogenated diarylanthracene derivative (Compound 6) can be obtained (Reaction Scheme 3).

In Reaction Scheme 3, X² and X³ each represent a halogen, and iodine and bromine are preferred as the halogen. Note that X² and X³ are preferred to be iodine and bromine, respectively, in order to suppress the homocoupling of Compound 5. In Reaction Scheme 3, R¹ to R⁸ each represent either hydrogen or an alkyl group having 1 to 4 carbon atoms. In Reaction Scheme 3, Ar¹ represents a substituted or unsubstituted aryl group having 6 to 13 carbon atoms. When Ar¹ has substituents, the substituents may be bonded to each other to form a ring. Furthermore, when a carbon atom of Ar¹ has two substituents, the substituents may be bonded to each other to form a spiro ring. In Reaction Scheme 3, Ar² represents a substituted or unsubstituted arylene group having 6 to 13 carbon atoms. When Ar² has substituents, the substituents may be bonded to each other to form a ring. Furthermore, when a carbon atom of Ar² has two substituents, the substituents may be bonded to each other to form a spiro ring. In this reaction, an organoboron compound which is a compound obtained by protecting the boronic acid of Compound 5 by ethylene glycol, pinacol, or the like may be used instead of Compound 5.

Examples of palladium catalysts which can be used in Reaction Scheme 3 include palladium(II)acetate, tetrakis(triphenylphosphine)palladium(0), and the like. Examples of ligands of palladium catalysts which can be used in Reaction Scheme 3 include tri(o-tolyl)phosphine, triphenylphosphine, tricyclohexylphosphine, and the like. Examples of bases which can be used in Reaction Scheme 3 include organic bases such as sodium tert-butoxide, inorganic bases such as potassium carbonate, and the like. Examples of solvents which can be used in Reaction Scheme 3 include a mixed solvent of toluene and water; a mixed solvent of toluene, alcohol such as ethanol, and water; a mixed solvent of xylene and water; a mixed solvent of xylene, alcohol such as ethanol, and water; a mixed solvent of benzene and water; a mixed solvent of benzene, alcohol such as ethanol, and water; a mixed solvent of ethers such as ethylene glycol dimethyl ether, and water; and the like. The use of a mixed solvent of toluene and water or a mixed solvent of toluene, ethanol, and water is more preferred.

Next, a synthetic method for the organic compound used in the synthesis of the anthracene derivatives in accordance with an embodiment of the present invention is described. Note that as described above, this organic compound is also an embodiment of the present invention.

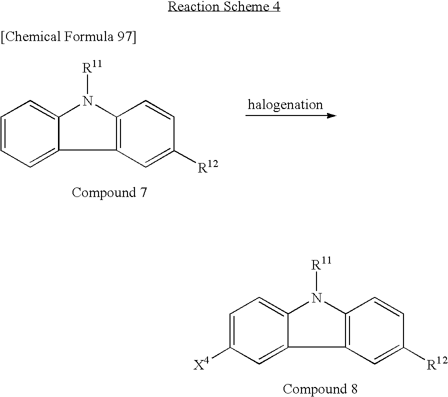

A carbazole derivative (Compound 7) is halogenated, whereby a halogenated carbazole derivative (Compound 8) can be obtained (Reaction Scheme 4).

In Reaction Scheme 4, X⁴ represents a halogen, and iodine, bromine, and chlorine are preferred as the halogen. In Reaction Scheme 4, R¹¹ represents either an alkyl group having 1 to 4 carbon atoms or a substituted or unsubstituted aryl group having 6 to 13 carbon atoms. When R¹¹ is an aryl group having substituents, the substituents may be bonded to each other to form a ring. Furthermore, when a carbon atom of R¹¹ has two substituents, the substituents may be bonded to each other to form a spiro ring. In Reaction Scheme 4, R¹² represents hydrogen, an alkyl group having 1 to 4 carbon atoms, or a substituted or unsubstituted aryl group having 6 to 13 carbon atoms. When R¹² is an aryl group having substituents, the substituents may be bonded to each other to form a ring. Furthermore, when a carbon atom of R¹² has two substituents, the substituents may be bonded to each other to form a spiro ring.

In the case where bromination of Compound 7 is carried out in Reaction Scheme 4, bromine, N-bromosuccinimide, and the like can be given as an example of a bromination agent. Examples of solvents which can be used in bromination with bromine include halogen-based solvents such as chloroform and carbon tetrachloride. Examples of solvents which can be used in bromination with N-bromosuccinimide are ethyl acetate, tetrahydrofuran, dimethylformamide, acetic acid, water, and the like.

In the case where iodination is carried out in Reaction Scheme 4, N-iodosuccinimide, 1,3-diiodo-5,5-dimethylimidazolidine-2,4-dione (abbreviated to DIH), 2,4,6,8-tetraiodo-2,4,6,8-tetraazabicyclo[3,3,0]octane-3,7-dione, 2-iodo-2,4,6,8-tetraazabicyclo[3,3,0]octane-3,7-dione, and the like can be used as an iodination reagent. When the iodination is carried out by using these iodination reagents, the following can be used alone or in combination: an aromatic hydrocarbon such as benzene, toluene, or xylene; an ether such as 1,2-dimethoxyethane, diethyl ether, methyl-t-butyl ether, tetrahydrofuran, or dioxane; a saturated hydrocarbon such as pentane, hexane, heptane, octane, or cyclohexane; a halogen such as dichloromethane, chloroform, tetrachloromethane, 1,2-dichloroethane, or 1,1,1-trichloroethane; a nitrile such as acetonitrile or benzonitrile; an ester such as ethyl acetate, methyl acetate, or butyl acetate; acetic acid (glacial acetic acid); water; or the like. When water is used, it is preferred to be mixed with an organic solvent. Moreover, it is preferred that the reaction be conducted in the presence of an acid such as sulfuric acid and acetic acid simultaneously.

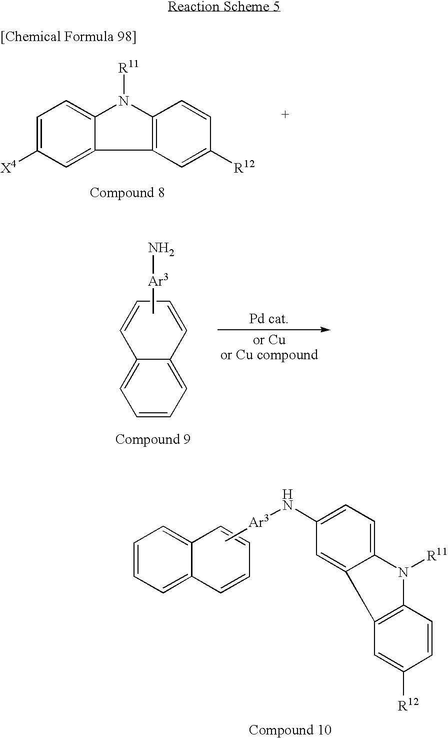

Next, coupling of the carbazole derivative (Compound 8) obtained by Reaction Scheme 4 and an arylamine derivative (Compound 9) by the Hartwig-Buchwald reaction using a palladium catalyst or the Ullmann reaction using copper or a copper compound is carried out, whereby an amine derivative (Compound 10) which is the organic compound in accordance with an embodiment of the present invention can be obtained (Reaction Scheme 5).

In Reaction Scheme 5, X⁴ represents a halogen, and iodine, bromine, and chlorine are preferred as the halogen. In Reaction Scheme 5, R¹¹ represents either an alkyl group having 1 to 4 carbon atoms or a substituted or unsubstituted aryl group having 6 to 13 carbon atoms. When R¹¹ is an aryl group having substituents, the substituents may be bonded to each other to form a ring. Furthermore, when a carbon atom of R¹¹ has two substituents, the substituents may be bonded to each other to form a spiro ring. In Reaction Scheme 5, R¹² represents hydrogen, an alkyl group having 1 to 4 carbon atoms, or a substituted or unsubstituted aryl group having 6 to 13 carbon atoms. When R¹² is an aryl group having substituents, the substituents may be bonded to each other to form a ring. Furthermore, when a carbon atom of R¹² has two substituents, the substituents may be bonded to each other to form a spiro ring. In Reaction Scheme 5, Ar³ represents a substituted or unsubstituted arylene group having 6 to 13 carbon atoms. When Ar³ has substituents, the substituents may be bonded to each other to form a ring. Furthermore, when a carbon atom of Ar³ has two substituents, the substituents may be bonded to each other to form a spiro ring.

In the case where the Hartwig-Buchwald reaction is carried out in Reaction Scheme 5, bis(dibenzylideneacetone)palladium(0), palladium(II) acetate, or the like can be used as the palladium catalyst. Examples of ligands of the palladium catalysts which can be used in Reaction Scheme 5 are tri(tert-butyl)phosphine, tri(n-hexyl)phosphine, tricyclohexylphosphine, and the like. Examples of bases which can be used in Reaction Scheme 5 are organic bases such as sodium tert-butoxide, inorganic bases such as potassium carbonate, and the like. The solvents which can be used in Reaction Scheme 5 are toluene, xylene, benzene, tetrahydrofuran, and the like.

The case of performing the Ullmann reaction in Reaction Scheme 5 is described. In Reaction Scheme 5, copper(I) iodide, copper(II) acetate, or the like can be used as the copper compound. Further, copper can be used in addition to the copper compound. Examples of bases which can be used in Reaction Scheme 5 are inorganic bases such as potassium carbonate, and the like. Examples of solvents which can be used in Reaction Scheme 5 are 1,3-dimethyl-3,4,5,6-tetrahydro-2(1H)pyrimidinone (abbreviated to DMPU), toluene, xylene, benzene, and the like. In the Ullmann reaction, since the target substance can be obtained in a shorter time and in a higher yield when the reaction temperature is 100° C. or higher, it is preferred to use DMPU or xylene which has a high boiling temperature. Since it is more preferred that the reaction temperature be 150° C. or higher, DMPU is more preferred to be used.

Next, a synthetic method of the anthracene derivative in accordance with an embodiment of the present invention is described.

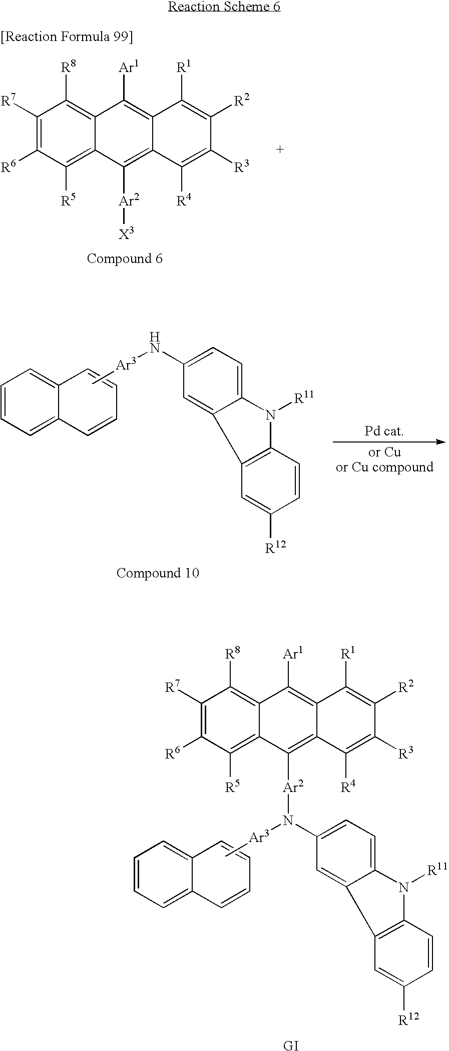

Coupling of the anthracene derivative (Compound 6) obtained by Reaction Scheme 3 and the amine derivative (Compound 10) obtained by Reaction Scheme 5 by the Hartwig-Buchwald reaction using a palladium catalyst or the Ullmann reaction using copper or a copper compound is carried out, whereby the target compound represented by General Formula (G1) can be obtained (Reaction Scheme 6).

In Reaction Scheme 6, X³ represents a halogen, and iodine, bromine, and chlorine are preferred as the halogen. In Reaction Scheme 6, Ar¹ represents a substituted or unsubstituted aryl group having 6 to 13 carbon atoms. When Ar¹ has substituents, the substituents may be bonded to each other to form a ring. Furthermore, when a carbon atom of Ar¹ has two substituents, the substituents may be bonded to each other to form a spiro ring. In Reaction Scheme 6, Ar² and Ar³ each independently represent a substituted or unsubstituted arylene group having 6 to 13 carbon atoms. When Ar² and Ar³ have substituents, the substituents may be bonded to each other to form a ring. Furthermore, when a carbon atom of Ar² and Ar³ has two substituents, the substituents may be bonded to each other to form a spiro ring. In Reaction Scheme 6, R¹ to R⁸ each represent either hydrogen or an alkyl group having 1 to 4 carbon atoms. In Reaction Scheme 6, R¹¹ represents either an alkyl group having 1 to 4 carbon atoms or a substituted or unsubstituted aryl group having 6 to 13 carbon atoms. When R¹¹ is an aryl group having substituents, the substituents may be bonded to each other to form a ring. Furthermore, when a carbon atom of R¹¹ has two substituents, the substituents may be bonded to each other to form a spiro ring. In Reaction Scheme 6, R¹² represents hydrogen, an alkyl group having 1 to 4 carbon atoms, or a substituted or unsubstituted aryl group having 6 to 13 carbon atoms. When R¹² is an aryl group having substituents, the substituents may be bonded to each other to form a ring. Furthermore, when a carbon atom of R¹² has two substituents, the substituents may be bonded to each other to form a spiro ring.

In the case where the Hartwig-Buchwald reaction is carried out in Reaction Scheme 6, bis(dibenzylideneacetone)palladium(0), palladium(II) acetate, or the like can be used as the palladium catalyst. Examples of ligands of the palladium catalysts which can be used in Reaction Scheme 6 are tri(tert-butyl)phosphine, tri(n-hexyl)phosphine, tricyclohexylphosphine, and the like. Examples of bases which can be used in Reaction Scheme 6 are organic bases such as sodium tert-butoxide, inorganic bases such as potassium carbonate, and the like. Examples of solvents which can be used in Reaction Scheme 6 are toluene, xylene, benzene, tetrahydrofuran, and the like.

The case of performing the Ullmann reaction in Reaction Scheme 6 is described. In Reaction Scheme 6, copper(I) iodide, copper(II) acetate, or the like can be used as the copper compound. Further, copper can be used in addition to the copper compound. Examples of bases which can be used in Reaction Scheme 6 are inorganic bases such as potassium carbonate, and the like. Examples of solvents which can be used in Reaction Scheme 6 are 1,3-dimethyl-3,4,5,6-tetrahydro-2(1H)pyrimidinone (abbreviated to DMPU), toluene, xylene, benzene, and the like. In the Ullmann reaction, since the target substance can be obtained in a shorter time and in a higher yield when the reaction temperature is 100° C. or higher, it is preferred to use DMPU or xylene which has a high boiling temperature. Since it is more preferred that the reaction temperature be 150° C. or higher, DMPU is more preferred to be used.

### Embodiment 2

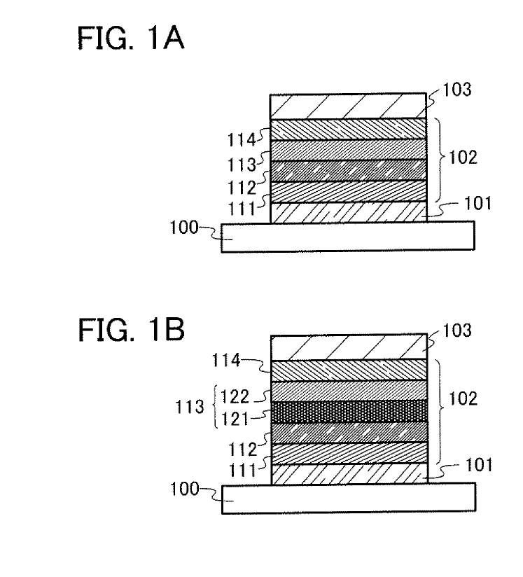

In this embodiment, an embodiment of a light-emitting element using the anthracene derivative described in Embodiment 1 is described with reference to FIG. 1A.

A light-emitting element of this embodiment has a plurality of layers between a pair of electrodes. The plurality of layers are formed by stacking layers including a substance having a high carrier injection property or a substance having a high carrier-transporting property so that a light-emitting region is formed apart from the electrodes, in other words, carriers are recombined in a portion apart from the electrodes.

In this embodiment, the light-emitting element illustrated in FIG. 1A includes a first electrode 101, a second electrode 103, and a layer 102 including an organic compound which is formed between the first electrode 101 and the second electrode 103. Note that in this embodiment, description is given below assuming that the first electrode 101 serves as an anode and the second electrode 103 serves as a cathode. That is, in the description below, it is assumed that light emission can be obtained when voltage is applied to the first electrode 101 and the second electrode 103 so that the potertial of the first electrode 101 is higher than that of the second electrode 103.

A substrate 100 is used as a support of the light-emitting element. For example, a glass substrate, a plastic substrate, or the like can be used as the substrate 100. Alternatively, the substrate 100 may be formed with any other material as long as the material can support the light-emitting element during a manufacturing process of the light-emitting element.

It is preferred that the first electrode 101 be formed using any of metals, alloys, and conductive compounds with a high work function (specifically, 4.0 eV or higher), a mixture thereof, or the like. Specifically, indium oxide-tin oxide (ITO: indium tin oxide), indium oxide-tin oxide containing silicon or silicon oxide, indium oxide-zinc oxide (IZO: indium zinc oxide), indium oxide containing tungsten oxide and zinc oxide (IWZO), or the like can be used. These conductive metal oxide films are generally formed by sputtering; however, the films may be formed by applying a sol-gel method, For example, indium oxide-zinc oxide (IZO) can be formed by a sputtering method using indium oxide to which 1 to 20 wt % of zinc oxide is added, as a target. Indium oxide containing tungsten oxide and zinc oxide (IWZO) can be formed by a sputtering method using a target in which 0.5 to 5 wt % of tungsten oxide and 0.1 to 1 wt % of zinc oxide are mixed with indium oxide. Alternatively, gold (Au), platinum (Pt), nickel (Ni), tungsten (W), chromium (Cr), molybdenum (Mo), iron (Fe), cobalt (Co), copper (Cu), palladium (Pd), a nitride of a metal (e.g., titanium nitride), or the like can be given.

When a layer including a composite material which is described later is used as a layer that is in contact with the first electrode 101, the first electrode 101 can be formed using any of a variety of metals, an alloy, a conductive compound, a mixture of them, or the like regardless of their work functions. For example, aluminum (Al), silver (Ag), an aluminum alloy (e.g., AlSi), or the like can be used. Further, an element belonging to Group 1 or Group 2 in the periodic table, which is a low work function material, that is, an alkali metal such as lithium (Li) or cesium (Cs), an alkaline earth metal such as magnesium (Mg), calcium (Ca), or strontium (Sr), an alloy including these metals (e.g., an MgAg alloy or an AlLi alloy), a rare earth metal such as europium (Eu) or ytterbium (Yb), an alloy including such rare earth metals, or the like can be used. A film of an alkali metal, an alkaline earth metal, or an alloy including these metals can be formed by a vacuum evaporation method. In addition, an alloy including an alkali metal or an alkaline earth metal can be formed by a sputtering method. Further, silver paste or the like can be applied by an ink-jet method to form the first electrode 101.

There is no particular limitation on the stack structure of the layer 102 including an organic compound. The layer 102 including an organic compound may have a structure in which one or more of layers including a substance having a high electron-transporting property, a substance having a high hole-transporting property, a substance having a high electron injection property, a substance having a high hole injection property, a bipolar substance (a substance having a high electron-transporting property and a high hole-transporting property), and/or the like is combined with the light-emitting layer described in this embodiment, as appropriate. For example, it is possible to combine a hole injection layer, a hole-transporting layer, a light-emitting layer, an electron-transporting layer, an electron injection layer, and the like to form the layer 102 including an organic compound. In this embodiment, description is given on a structure in which the layer 102 including an organic compound comprises a hole injection layer 111, a hole-transporting layer 112, a light-emitting layer 113, and an electron-transporting layer 114 stacked in that order over the first electrode 101. Specific materials to form each of the layers are given below.

The hole injection layer 111 is a layer including a substance having a high hole injection property. Molybdenum oxide, vanadium oxide, ruthenium oxide, tungsten oxide, manganese oxide, and the like can be used for the hole injection layer 111. Alternatively, the hole injection layer 111 can be formed using any one of the following materials: phthalocyanine based compounds such as phthalocyanine (abbreviated to H₂Pc) and copper phthalocyanine (abbreviated to CuPc); aromatic amine compounds such as 4,4′-bis[N-(4-diphenylaminophenyl)-N-phenylamino]biphenyl (abbreviated to DPAB) and 4,4′-bis(N-{4-[N-(3-methylphenyl)-N-phenylamino]phenyl}-N-phenylamino)biphenyl (abbreviated to DNTPD); high molecular compounds such as poly(ethylenedioxythiophene)/poly(styrenesulfonic acid) (abbreviated to PEDOT/PSS); and the like. Further, the hole injection layer 111 can be formed using a tris(p-enamine-substituted-aminophenyl)amine compound, a 2,7-diamino-9-fluorenylidene compound, a tri(p-N-enamine-substituted-aminophenyl)benzene compound, a pyrene compound having one or two ethenyl groups having at least one aryl group, N,N′-di(biphenyl-4-yl)-N,N′-diphenylbiphenyl-4,4′-diamine, N,N,N′,N′-tetra(biphenyl-4-yl)biphenyl-4,4′-diamine, N,N,N′,N′-tetra(biphenyl-4-yl)-3,3′-diethylbiphenyl-4,4′-diamine, 2,2′-(methylenedi-4,1-phenylene)bis[4,5-bis(4-methoxyphenyl)-2H-1,2,3-triazole], 2,2′-(biphenyl-4,4′-diyl)bis(4,5-diphenyl-2H-1,2,3-triazole), 2,2′-(3,3′-dimethylbipheny-4,4′-diyl)bis(4,5-diphenyl-2H-1,2,3-triazole), bis[4-(4,5-diphenyl-2H-1,2,3-triazol-2-yl)phenyl](methyl)amine, or the like.

As a further alternative, a composite material formed by mixing an acceptor substance into a substance having a high hole-transporting property can also be used for the hole injection layer 111. Note that by using a composite containing the substance having a high hole-transporting property and an acceptor substance, a material used to form an electrode may be selected regardless of its work function. That is, not only a high work function material, but also a low work function material can be used for the first electrode 101. As the acceptor substance, 7,7,8,8-tetracyano-2,3,5,6-tetrafluoroquinodimethane (abbreviated to F₄-TCNQ), chloranil, and the like can be given. In addition, a transition metal oxide is given. Furthermore, an oxide of metals that belong to Group 4 to Group 8 of the periodic table can be given. Specifically, vanadium oxide, niobium oxide, tantalum oxide, chromium oxide, molybdenum oxide, tungsten oxide, manganese oxide, and rhenium oxide are preferred because of a high electron-accepting property. Among these metal oxides, molybdenum oxide is especially preferred since it is stable in the air and its hygroscopic property is low so that it can be easily treated.

As a substance having a high hole-transporting property used for the composite material, various compounds such as aromatic amine compounds, carbazole derivatives, aromatic hydrocarbons, and high molecular compounds (e.g., an oligomer, a dendrimer, or a polymer) can be used. Note that it is preferred that the substance having a hole mobility of 10⁻⁶ cm²/Vs or higher be used as the substance having a high hole-transporting property used for the composite material. Note that a substance other than the above substances may be used as long as it has a hole-transporting property higher than an electron-transporting property. The organic compound that can be used for the composite material is specifically listed below.

Examples of the aromatic amine compounds that can be used for the composite material include N,N′-bis(4-methylphenyl)(p-tolyl)-N,N′-diphenyl-p-phenylenediamine (abbreviated to DTDPPA), 4,4′-bis[N-(4-diphenylaminophenyl)-N-phenylamino]biphenyl (abbreviated to DPAB), N,N′-bis[4-[bis(3-methylphenyl)amino]phenyl]-N,N′-diphenyl-[1,1′-biphenyl]-4,4′-diamine (abbreviated to DNTPD), 1,3,5-tris[N-(4-diphenylaminophenyl)-N-phenylamino]benzene (abbreviated to DPA3B), and the like.

Specific examples of the carbazole derivatives that can be used for the composite material include 3-[N-(9-phenylcarbazol-3-yl)-N-phenylamino]-9-phenylcarbazole (abbreviated to PCzPCA1), 3,6-bis[N-(9-phenylcarbazol-3-yl)-N-phenylamino]-9-phenylcarbazole (abbreviated to PCzPCA2), 3-[N-(1-naphthyl)-N-(9-phenylcarbazol-3-yl)amino]-9-phenylcarbazole (abbreviated to PCzPCN1), and the like.

Further, the carbazole derivatives that can be used for the composite material include 4,4′-di(N-carbazolyl)biphenyl (abbreviated to CBP), 1,3,5-tris[4-(N-carbazolyl)phenyl]benzene (abbreviated to TCPB), 9-[4-(10-phenyl-9-anthracenyl)phenyl]-9H-carbazole (abbreviated to CzPA), 1,4-bis[4-(N-carbazolyl)phenyl]-2,3,5,6-tetraphenylbenzene, and the like.

As the aromatic hydrocarbons which can be used for the composite material, the following can be given for example: 2-tert-butyl-9,10-di(2-naphthyl)anthracene (abbreviated to t-BuDNA); 2-tert-butyl-9,10-di(1-naphthyl)anthracene, 9,10-bis(3,5-diphenylphenyl)anthracene (abbreviated to DPPA); 2-tert-butyl-9,10-bis(4-phenylphenyl)anthracene (abbreviated to t-BuDBA); 9,10-di(2-naphthyl)anthracene (abbreviated to DNA); 9,10-diphenylanthracene (abbreviated to DPAnth); 2-tert-butylanthracene (abbreviated to t-BuAnth); 9,10-bis(4-methyl-1-naphthyl)anthracene (abbreviated to DMNA); 9,10-bis[2-(1-naphthyl)phenyl]-2-tert-butyl-anthracene; 9,10-bis[2-(1-naphthyl)phenyl]anthracene; 2,3,6,7-tetramethyl-9,10-di(1-naphthyl)anthracene; 2,3,6,7-tetramethyl-9,10-di(2-naphthyl)anthracene; 9,9′-bianthryl; 10,10′-diphenyl-9,9′-bianthryl; 10,10′-bis(2-phenylphenyl)-9,9′-bianthryl; 10,10′-bis[(2,3,4,5,6-pentaphenyl)phenyl]-9,9′-bianthryl; anthracene; tetracene; rubrene; perylene; 2,5,8,11-tetra(tert-butyl)perylene; and the like. Alternatively, pentacene, coronene, or the like can also be used. Thus, the aromatic hydrocarbon which has a hole mobility of 1×10⁻⁶ cm²/Vs or higher and which has 14 to 42 carbon atoms is particularly preferred.

The aromatic hydrocarbons that can be used for the composite material may have a vinyl skeleton. As an aromatic hydrocarbon having a vinyl group, for example, 4,4′-bis(2,2-diphenylvinyl)biphenyl (abbreviated to DPVBi), 9,10-bis[4-(2,2-diphenylvinyl)phenyl]anthracene (abbreviated to DPVPA), and the like are given.

For the hole injection layer 111, a high molecular compound (e.g., an oligomer, a dendrimer, or a polymer) can be used. For example, the following high molecular compound can be used: poly(N-vinylcarbazole) (abbreviated to PVK); poly(4-vinyltriphenylamine) (abbreviated to PVTPA); poly[N-(4-[N′-[4-(4-diphenylamino)phenyl]phenyl-N′-phenylamino]phenyl)methaclylamide] (abbreviated to PTPDMA); poly[N,N′-bis(4-butylphenyl)-N,N′-bis(phenyl)benzidine] (abbreviated to Poly-TPD); and the like. In addition, high molecular compounds doped with acid such as poly(3,4-ethylenedioxythiophene)/poly(styrenesulfonic acid) (PEDOT/PSS), polyaniline/poly(styrenesulfonic acid) (PAni/PSS) can be used.

Note that the hole injection layer 111 can be formed using a composite material of the above-described high molecular compound, such as PVK, PVTPA, PTPDMA, or Poly-TPD, and the above-described acceptor substance.

The hole-transporting layer 112 is a layer including a substance having a high hole-transporting property. As the substance having a high hole-transporting property, for example, an aromatic amine compound such as 4,4′-bis[N-(1-naphthyl)-N-phenylamino]biphenyl (abbreviated to NPB), N,N′-bis(3-methylphenyl)-N,N′-diphenyl-[1,1′-biphenyl]-4,4′-diamine (abbreviated to TPD), 4,4′,4″-tris(N,N-diphenylamino)triphenylamine (abbreviated to TDATA), 4,4′,4″-tris[N-(3-methyl-phenyl)-N-phenylamino]triphenylamine (abbreviated to MTDATA), or 4,4′-bis[N-(spiro-9,9′-bifluoren-2-yl)-N-phenylamino]biphenyl (abbreviated to BSPB) can be used. The materials described here are mainly materials having a hole mobility of 10⁻⁶ cm²/Vs or higher. Note that a substance other than the above substances may be used as long as it has a hole-transporting property higher than an electron-transporting property. Further, the layer including a substance with a high hole-transporting property may be not only a single layer but also a stacked layer of two or more layers using the above-mentioned substances.

For the hole-transporting layer 112, a high molecular compound such as PVK, PVTPA, PTPDMA, or Poly-TPD can be used alternatively. Further, the hole-transporting layer 112 can be formed using a tris(p-enamine-substituted-aminophenyl)amine compound, a 2,7-diamino-9-fluorenylidene compound, a tri(p-N-enamine-substituted-aminophenyl)benzene compound, a pyrene compound having one or two ethenyl groups having at least one aryl group, N,N′-di(biphenyl-4-yl)-N,N′-diphenylbiphenyl-4,4′-diamine, N,N,N′,N′-tetra(biphenyl-4-yl)biphenyl-4,4′-diamine, N,N,N′,N′-tetra(biphenyl-4-yl)-3,3′-diethylbiphenyl-4,4′-diamine, 2,2′-(methylenedi-4,1-phenylene)bis[4,5-bis(4-methoxyphenyl)-2H-1,2,3-triazole], 2,2′-(biphenyl-4,4′-diyl)bis(4,5-diphenyl-2H-1,2,3-triazole), 2,2′-(3,3′-dimethylbipheny-4,4′-diyl)bis(4,5-diphenyl-2H-1,2,3-triazole), bis[4-(4,5-diphenyl-2H-1,2,3-triazol-2-yl)phenyl](methyl)amine, or the like.

The light-emitting layer 113 is a layer including a substance having a high light-emitting property. In the light-emitting element of this embodiment, the light-emitting layer 113 is formed using any of the anthracene derivatives described in Embodiment 1. These anthracene derivatives are suitable for the use in a light-emitting element as a substance having a high light-emitting property because they exhibit high emission efficiency.

The electron-transporting layer 114 is a layer including a substance having a high electron-transporting property. For example, it is possible to employ a metal complex or the like having a quinoline or benzoquinoline skeleton, such as tris(8-quinolinolato)aluminum (abbreviated to Alq), tris(4-methyl-8-quinolinolato)aluminum (abbreviated to Almq₃), bis(10-hydroxybenzo[h]quinolinato)beryllium (abbreviated to BeBq₂), or bis(2-methyl-8-quinolinolato)(4-phenylphenolato)aluminum (abbreviated to BAlq) for the electron-transporting layer 114. Alternatively, a metal complex having an oxazole-based or thiazole-based ligand, such as bis[2-(2-hydroxyphenyl)benzoxazolato]zinc (abbreviated to Zn(BOX)₂) or bis[2-(2-hydroxyphenyl)benzothiazolato]zinc (abbreviated to Zn(BTZ)₂) can be used. Besides the metal complexes, 2-(4-biphenyl)-1)-5-(4-tert-butylphenyl)-1,3,4-oxadiazole (abbreviated to PBD), 1,3-bis[5-(p-tert-butylphenyl)-1,3,4-oxadiazole-2-yl]benzene (abbreviated to OXD-7), 3-(4-biphenyl)-1)-4-phenyl-5-(4-tert-butylphenyl)-1,2,4-triazole (abbreviated to TAZ), bathophenanthroline (abbreviated to BPhen), bathocuproine (abbreviated to BCP), bis[3-(1H-benzimidazol-2-yl)fluoren-2-olato]zinc(II), bis[3-(1H-benzimidazol-2-yl)fluoren-2-olato]beryllium(II), bis[2-(1H-benzimidazol-2-yl)dibenzo[b,d]furan-3-olato] (phenolato)aluminum(III), bis[2-(benzoxazol-2-yl)-7,8-methylenedioxydibenzo[b,d]furan-3-olato] (2-naphtholato)aluminum(III), or the like can also be used. The substances described here are mainly substances having electron mobility of 10⁻⁶ cm²/Vs or higher. Note that a substance other than the above substances may be used as long as it has an electron-transporting property higher than a hole-transporting property. Further, the electron-transporting layer may be not only a single layer but also a stacked layer of two or more layers using the above-mentioned substances.

For the electron-transporting layer 114, a high molecular compound can be used. For example, poly[(9,9-dihexylfluorene-2,7-diyl)-co-(pyridine-3,5-diyl)] (abbreviated to PF-Py), poly[(9,9-dioctylfluorene-2,7-diyl)-co-(2,2′-bipyridine-6,6′-diyl)] (abbreviated to PF-BPy), and the like can be used.

An electron injection layer may be provided between the electron-transporting layer 114 and the second electrode 103. The electron injection layer can be formed using an alkali metal compound or an alkaline earth metal compound such as lithium fluoride (LiF), cesium fluoride (CsF), or calcium fluoride (CaF₂). Further, a layer in which a substance having an electron-transporting property is combined with an alkali metal or an alkaline earth metal can be employed. For instance, a layer comprising Alq to which magnesium (Mg) is added can be used. It is more preferred to use the layer in which a substance having an electron-transporting property is combined with an alkali metal or an alkaline earth metal as the electron injection layer because electron injection from the second electrode 103 efficiently proceeds.

It is preferred that the second electrode 103 be formed using a metal, an alloy, or a conductive compound with a low work function (specifically, 3.8 eV or lower), a mixture of them, or the like. Specific examples of such cathode materials include elements belonging to Group 1 or Group 2 of the periodic table, i.e., alkali metals such as lithium (Li) and cesium (Cs) and alkaline earth metals such as magnesium (Mg), calcium (Ca), and strontium (Sr); alloys of them (e.g. MgAg and AlLi); rare earth metals such as europium (Eu) and ytterbium (Yb), alloys of them; and the like. A film using an alkali metal, an alkaline earth metal, or an alloy including these metals can be formed by a vacuum evaporation method. In addition, an alloy including an alkali metal or an alkaline earth metal can be formed by a sputtering method. Further, silver paste or the like can be applied by an ink-jet method to form the second electrode 103.

When the electron injection layer is provided between the second electrode 103 and the electron-transporting layer 114, any of a variety of conductive materials such as Al, Ag, ITO, and indium oxide-tin oxide containing silicon or silicon oxide can be used for the second electrode 103 regardless of its work function. These conductive materials can be formed by a sputtering method, an ink-jet method, a spin coating method, or the like.

In the light-emitting element described in this embodiment, which has the above-mentioned structure, current flows by application of voltage between the first electrode 101 and the second electrode 103. Then, holes and electrons are recombined in the light-emitting layer 113 which is a layer including a substance having a high light-emitting property. That is, the light-emitting element has a structure in which a light-emitting region is formed in the light-emitting layer 113.

Light is extracted outside through one or both of the first electrode 101 and the second electrode 103. Thus, one or both of the first electrode 101 and the second electrode 103 is/are light-transmitting electrodes. When only the first electrode 101 is a light-transmitting electrode, light is extracted from the substrate 100 side through the first electrode 101. In contrast, when only the second electrode 103 is a light-transmitting electrode, light is extracted from the side opposite to the substrate 100 side through the second electrode 103. When both the first electrode 101 and the second electrode 103 are light-transmitting electrodes, light is extracted from both the substrate 100 side and the side opposite to the substrate 100 side through the first electrode 101 and the second electrode 103.

Although in FIG. 1A, a structure in which the first electrode 101 which serves as an anode is provided on the substrate 100 side is illustrated, the second electrode 103 which serves as a cathode may be provided on the substrate 100 side. Note that in this case, it is preferred that a TFT connected to the second electrode 103 be an n-channel TFT.

Any of a variety of methods can be employed for forming the layer 102 including an organic compound regardless of whether it is a dry process or a wet process. Moreover, a different forming method may be used for each electrode or each layer. A vacuum evaporation method, a sputtering method, or the like can be employed as a dry process. An ink-jet method, a spin-coating method, or the like can be employed as a wet process.

Further, the electrodes may be formed by a sol-gel method, which is a wet process, or may also be formed by a wet process using a paste of a metal material. Further, the electrode may be formed by a dry method such as a sputtering method or a vacuum evaporation method.

Hereinafter, specific methods for forming a light-emitting element are described. When the light-emitting element in accordance with an embodiment of the present invention is applied to a display device and its light-emitting layer is separately coated, it is preferred that the light-emitting layer be formed by a wet process. By the use of a wet process such as an ink-jet method, separate coloring for the light-emitting layers is easy even if a large substrate is employed, whereby the productivity is improved.

For example, in the structure described in this embodiment, the first electrode 101 may be formed by a sputtering method, which is a dry process; the hole injection layer 111 may be formed by an ink-jet method or a spin coating method, which are wet processes; the hole-transporting layer 112 may be formed by a vacuum evaporation method, which is a dry process; the light-emitting layer 113 may be formed by an ink-jet method, which is a wet process; the electron-transporting layer 114 may be formed by a co-deposition method, which is a dry process; and the second electrode 103 may be formed by an ink-jet method or a spin coating method, which are wet processes. Further, the first electrode 101 may be formed by an ink-jet method, which is a wet process; the hole injection layer 111 may be formed by a vacuum evaporation method, which is a dry process; the hole-transporting layer 112 may be formed by an ink-jet method or a spin coating method, which are wet processes; the light-emitting layer 113 may be formed by an ink-jet method, which is a wet process; the electron-transporting layer 114 may be formed by an ink-jet method or a spin coating method, which are wet processes; and the second electrode 103 may be formed by an ink-jet method or a spin coating method, which are wet processes. The method for forming the light-emitting element is not particularly limited to the above methods, and a wet method and a dry method may be combined as appropriate.

For example, the first electrode 101 may be formed by a sputtering method which is a dry process; the hole injection layer 111 and the hole-transporting layer 112 may be formed by an ink-jet method or a spin coating method, which are wet processes; the light-emitting layer 113 may be formed by an ink-jet method which is a wet process; the electron-transporting layer 114 may be formed by a vacuum evaporation method, which is a dry process; and the second electrode 103 may be formed by a vacuum evaporation method which is a dry process. That is, it is possible to form the hole injection layer 111 to the light-emitting layer 113 by wet processes and to form the electron-transporting layer 114 to the second electrode 103 by dry processes over the substrate 100 over which the first electrode 101 is formed in a desired shape. By this method, the hole injection layer 111 to the light-emitting layer 113 can be formed at atmospheric pressure, and separate coloring for the light-emitting layer 113 is easy. In addition, the electron-transporting layer 114 to the second electrode 103 can be formed in vacuum consistertly. Therefore, the processes can be simplified, and the productivity can be improved.

When the light-emitting layer 113 is formed by a wet process, a liquid composition in which any of the anthracene derivatives described in Embodiment 1 is dissolved in a solvent can be used. In this case, the liquid composition comprising any of the anthracene derivatives described in Embodiment 1 and a solution is applied to a region where the light-emitting layer 113 is to be formed, the solvent is then removed by heat treatment or the like, and the anthracene derivative described in Embodiment 1 is solidified, whereby a thin film of the light-emitting layer 113 is formed.

As regards the light-emitting element having the above-mentioned structure, current flows when a potertial difference is given between the first electrode 101 and the second electrode 103, and then holes and electrons are recombined in the light-emitting layer 113, which is the layer including a substance having a high light-emitting property, resulting in emission of light. That is, the light-emitting element has a structure in which a light-emitting region is formed in the light-emitting layer 113.

Note that a structure of the layers provided between the first electrode 101 and the second electrode 103 is not limited to the structure described above. Any structure other than the above structure can be employed as long as a light-emitting region in which holes and electrons are recombined is provided away from the first electrode 101 and the second electrode 103 in order to prevent quenching of emitted light which is caused by the approach of the light-emitting region to a metal.

For example, a structure may be employed in which a hole-transporting layer is not provided and an electron-injection suppression layer is provided for suppressing injection of electrons from the hole injection layer including an acceptor and a light-emitting layer. In that case, it is preferred that the electron affinity of a material for forming the electron-injection suppression layer be smaller than that of a material for forming the light-emitting layer and the acceptor. Alternatively, a structure may be employed in which not an electron-transporting layer but a hole-injection suppression layer is provided for suppressing injection of holes from the electron injection layer and from the light-emitting layer. In that case, it is preferred that the ionization potertial of a material for forming the hole-injection suppression layer be larger than that of a material for forming the light-emitting layer and the donor.

Further, a light-emitting element described in this embodiment may have a structure in which two or more layers of the hole injection layer 111 and two or more layers of the hole-transporting layer 112 described above are alternately stacked. Further, the electrode which serves as a cathode may have a three-layer structure in which a second metal electrode which prevents oxidation is interposed between an oxide transparent conductive film and a metal electrode.

The anthracene derivatives described in Embodiment 1 have high emission efficiency; thus, as described in this embodiment, they can be used for a light-emitting layer without adding any other light-emitting substance. Furthermore, since the anthracene derivatives described in Embodiment 1 have high emission efficiency, a light-emitting element with high emission efficiency can be obtained.

Since the anthracene derivatives described in Embodiment 1 emit blue light with excellent color purity, a light-emitting element which exhibits blue light emission with excellent color purity can be obtained.

Additionally, since the anthracene derivatives described in Embodiment 1 are able to emit blue light with excellent color purity at high efficiency, a light-emitting element which can emit blue light with high luminous efficiency can be obtained.

Further, with use of the anthracene derivatives described in Embodiment 1, a blue emissive light-emitting element having a long lifetime can be obtained.

Further, since a light-emitting element using any of the anthracene derivatives described in Embodiment 1 can emit blue light at high efficiency, the light-emitting element is suitable for use in a full-color display. Moreover, the light-emitting element can emit blue light for a long period of time; therefore, the light-emitting element is suitable for use in a full-color display. In particular, since blue emissive light-emitting elements are less developed in terms of lifetime and efficiency than green emissive or red emissive light-emitting elements, high-performance blue emissive light-emitting elements have been desired. A light-emitting element using any of the anthracene derivatives described in Embodiment 1 is capable of emitting blue light at high efficiency and exhibiting a long lifetime and thus is suitable for a full-color display.

### Embodiment 3

In this embodiment, a light-emitting element having a different structure from that described in Embodiment 2 is described.

The light-emitting layer 113 described in Embodiment 2 is formed by dispersing the anthracene derivative described in Embodiment 1 into another substance, whereby light emission can be obtained from the anthracene derivative described in Embodiment 1. With the anthracene derivative described in Embodiment 1 which emits blue light, a blue emissive light-emitting element can be obtained.

Here, as the substance in which the anthracene derivative described in Embodiment 1 is dispersed, in addition to the substance with a high hole-transporting property and the substance with a high electron-transporting property described in Embodiment 2, other various materials can be used such as 4,4′-bis(N-carbazolyl)-biphenyl (abbreviated to CBP), 2,2′,2″-(1,3,5-benzenetriyl)tris[1-phenyl-1H-benzimidazole] (abbreviated to TPBI), 9,10-di(2-naphthyl)anthracene (abbreviated to DNA), 2-tert-butyl-9,10-di(2-naphthyl)anthracene (abbreviated to t-BuDNA), 9-[4-(N-carbazolyl)phenyl]-10-phenylanthracene (abbreviated to CzPA). Further, as the substance in which the anthracene derivative described in Embodiment 1 is dispersed, a high molecular compound can be used. For example, poly(N-vinylcarbazole) (abbreviated to PVK); poly(4-vinyltriphenylamine) (abbreviated to PVTPA); poly[N-(4-{N′-[4-(4-diphenylamino)phenyl]phenyl-N′-phenylamino}phenyl)methacrylamide] (abbreviated to PTPDMA); poly[N,N′-bis(4-butylphenyl)-N,N′-bis(phenyl)benzidine] (abbreviated to Poly-TPD); poly[(9,9-dihexylfluorene-2,7-diyl)-co-(pyridine-3,5-diyl)] (abbreviated to PF-Py); poly[(9,9-dioctylfluorene-2,7-diyl)-co-(2,2′-bipyridine-6,6′-diyl)] (abbreviated to PF-BPy); or the like can be used. Alternatively, it is possible to use a compound in which six or more aryl groups are substituted at terphenyl, 4,4′-bis(2,2-diphenylvinyl)-1,1′-binaphthyl, 4,4′-bis[2,2-bis(4-methylphenyl)vinyl]-1,1′-binaphthyl, 4,4′-bis[2,2-bis(4-methoxyphenyl)vinyl]-1,1′-binaphthyl, 4,4′-bis(2-methyl-2-phenylvinyl)-1,1′-binaphthyl, 4,4′-distyryl-1,1′-binaphthyl, 4,4′-bis[2-(2-naphtyl)-2-phenylvinyl]-1,1′-binaphthyl, 4,4′-bis[2-(1-naphtyl)-2-phenylvinyl]-1,1′-binaphthyl, 4,4′-bis[2-(biphenyl-4-yl)-2-phenylvinyl]-1,1′-binaphthyl, bis[3-(1H-benzimidazol-2-yl)fluoren-2-olato]zinc(II), bis[3-(1H-benzimidazol-2-yl)fluoren-2-olato]beryllium(II), bis[2-(1H-benzimidazol-2-yl)dibenzo[b,d]furan-3-olato](phenolato)aluminum(III), bis[2-(benzoxazol-2-O-7,8-methylenedioxydibenzo[b,d]furan-3-olato] (2-naphtholato)aluminum(III), or the like.

Since the anthracene derivatives described in Embodiment 1 have high emission efficiency, a light-emitting element with high emission efficiency can be obtained by using any of the anthracene derivatives for a light-emitting element.

Since the anthracene derivatives described in Embodiment 1 emit blue light with high color purity, a light-emitting element which emits blue light with high color purity can be obtained.

Further, the anthracene derivatives described in Embodiment 1 emit light at high efficiency, and thus a light-emitting element that can emit blue light with high luminous efficiency can be obtained.

Further, with the use of any of the anthracene derivatives described in Embodiment 1, a light-emitting element having a long lifetime can be obtained.

Since the light-emitting element using any of the anthracene derivatives described in Embodiment 1 can emit blue light with high color purity at high efficiency, the light-emitting element is suitable for use in a full-color display. Further, since the light-emitting element can emit blue light with a long lifetime, the light-emitting element is suitable for the use in a full-color display.

In addition, in the light-emitting layer 113, not only the anthracene derivatives but also a substance doped with an alkali metal salt of a carboxyl acid having a pyridine ring, a substance doped with a pyridine derivative including an alkali metal, or a substance doped with an alkali metal salt of a phenol compound is used, whereby a light-emitting element which can be driven at a low voltage can be realized in addition to the above-described effects.

Note that, regarding the layers other than the light-emitting layer 113, the structure described in Embodiment 2 can be used as appropriate. In addition, other description is based on Embodiment 2.

### Embodiment 4

In this embodiment, a light-emitting element having a different structure from those described in Embodiments 2 and 3 is described.

The light-emitting layer 113 described in Embodiment 2 is formed by dispersing a light-emitting substance in the anthracene derivative described in Embodiment 1, whereby light emission from the light-emitting substance can be obtained.

When the anthracene derivative described in Embodiment 1 is used as a material in which another light-emitting substance is dispersed, emission color resulting from the light-emitting substance can be obtained. Alternatively, a mixed emission color of an emission color resulting from the anthracene derivative described in Embodiment 1 and an emission color resulting from the light-emitting substance dispersed in the anthracene derivative can be obtained.

Here, a variety of materials can be used as the light-emitting substance dispersed in the anthracene derivative described in Embodiment 1. Specifically, the following fluorescent substances which emit fluorescence can be used: N,N″-diphenylquinacridone (abbreviated to DPQd), coumarin 6, coumarin 545T, 4-(dicyanomethylene)-2-methyl-6-(p-dimethylaminostyryl)-4H-pyran (abbreviated to DCM1), 4-(dicyanomethylene)-2-methyl-6-(julolidin-4-yl-vinyl)-4H-pyran (abbreviated to DCM2), N,N″-dimethylquinacridone (abbreviated to DMQd), {2-(1,1-dimethylethyl)-6-[2-(2,3,6,7-tetrahydro-1,1,7,7-tetramethyl-1H,5H-benzo[ij]quinolizin-9-yl)ethenyl]-4H-pyran-4-ylidene}propanedinitrile (abbreviated to DCJTB), 5,12-diphenyltetracene (abbreviated to DPT), N,9-diphenyl-N-[4-(10-phenyl-9-anthryl)phenyl]-9H-carbazol-3-amine (abbreviated to PCAPA), N,N″-(2-tert-butylanthracene-9,10-diyldi-4,1-phenylene)bis[N,N′,N′-triphenyl-1,4-phenylenediamine] (abbreviated to DPABPA), N,N″-diphenyl-N,N′-bis(9-phenylcarbazol-3-yl)stilbene-4,4′-diamine (abbreviated to PCA2S), 2,5,8,11-tetra(tert-butyl)perylene (abbreviated to TBP), perylene, rubrene, 1,3,6,8-tetraphenylpyrene, bis[3-(1H-benzimidazol-2-yl)fluoren-2-olato]zinc(II), bis[3-(1H-benzimidazol-2-yl)fluoren-2-olato]beryllium(II), bis[2-(1H-benzimidazol-2-yl)dibenzo[b,d]furan-3-olato](phenolato)aluminum(III), bis[2-(benzoxazol-2-yl)-7,8-methylenedioxydibenzo[b,d]furan-3-olato](2-naphtholato)aluminum(III), and the like. Besides, it is possible to use a compound in which six or more aryl groups are substituted at terphenyl. Further, phosphorescent substances that emit phosphorescence such as (acetylacetonato)bis[2,3-bis(4-fluorophenyl)quinoxalinato]iridium(III) (abbreviated to Ir(Fdpq)₂(acac)) and 2,3,7,8,12,13,17,18-octaethyl-21H,23H-porphyrinplatinum(II) (abbreviated to PtOEP) can be used.

Note that, regarding the layers other than the light-emitting layer 113, the structure described in Embodiment 2 can be used as appropriate. In addition, other description is based on Embodiment 2.

### Embodiment 5

In this embodiment, a light-emitting element having a structure different from those described in Embodiments 2 to 4 is described with reference to FIG. 1B.

In the light-emitting element described in this embodiment, a first layer 121 and a second layer 122 are provided in the light-emitting layer 113 of the light-emitting element described in Embodiment 2.

The light-emitting layer 113 is a layer including a substance having a high light-emitting property. In the light-emitting element described in this embodiment, the light-emitting layer 113 has the first layer 121 and the second layer 122. The first layer 121 includes a first organic compound and an organic compound having a hole-transporting property, and the second layer 122 includes a second organic compound and an organic compound having an electron-transporting property. The first layer 121 is provided in contact with the first electrode side of the second layer 122, that is, in contact with the anode side.

Both the first organic compound and the second organic compound are substances having a high light-emitting property. In the light-emitting element described in this embodiment, the first organic compound or the second organic compound contains any of the anthracene derivatives described in Embodiment 1. Since the anthracene derivatives described in Embodiment 1 emit blue green to blue light, the anthracene derivatives are each suitable for use as a substance having a high light-emitting property in the light-emitting element described in this embodiment. Note that the first organic compound and the second organic compound may be the same or different from each other.

When any of the anthracene derivatives described in Embodiment 1 is used as one of the first organic compound and the second organic compound, as the other one thereof, it is possible to use substances that emit blue light, such as 4-(9H-carbazol-9-yl)-4′-(10-phenyl-9-anthryl)triphenylamine (abbreviated to YGAPA), 4,4′(2-tert-butylanthracen-9,10-diyl)bis{N-[4-(9H-carbazol-9-yl)phenyl]-N-phenylaniline} (abbreviated to YGABPA), N,9-diphenyl-N-[4-(10-phenyl-9-anthryl)phenyl]-9H-carbazol-3-amine (abbreviated to PCAPA), N,N″-(2-tert-butylanthracene-9,10-diyldi-4,1-phenylene)bis[N,N′,N″-triphenyl-1,4-phenylenediamine] (abbreviated to DPABPA), N,N′-bis[4-(9H-carbazol-9-yl)phenyl]-N,N-diphenylstilbene-4,4′-diamine (abbreviated to YGA2S), N-[4-(9H-carbazol-9-yl)phenyl]-N-phenylstilbene-4-amine (abbreviated to YGAS), N,N-diphenyl-N,N-bis(9-phenylcarbazol-3-yl)stilbene-4,4′-diamine (abbreviated to PCA2S), 4,4′-bis(2,2-diphenylvinyl)biphenyl (abbreviated to DPVBi), 2,5,8,11-tetra(tert-butyl)perylene (abbreviated to TBP), perylene, rubrene, and 1,3,6,8-tetraphenylpyrene. Since each of these substances emits light with a color which is similar to that of the anthracene derivatives described in Embodiment 1, they are suitable for use in the light-emitting element of this embodiment.

The organic compound having a hole-transporting property, which is included in the first layer 121, is a substance having a hole-transporting property higher than an electron-transporting property. The organic compound having an electron-transporting property included in the second layer 122 is a substance having an electron-transporting property higher than a hole-transporting property.

The mechanism of the light-emitting element of this embodiment having the above-described structure is described with reference to FIG. 1B.

In FIG. 1B, holes are injected from the first electrode 101 into the first layer 121 of the light-emitting layer 113 through the hole injection layer 111 and the hole-transporting layer 112. The holes injected into the first layer 121 are transported through the first layer 121 and further injected into the second layer 122. Here, the organic compound having an electron-transporting property which is included in the second layer 122 is a substance which has an electron-transporting property higher than a hole-transporting property, and thus, the holes injected into the second layer 122 are difficult to move. Consequently, a large number of holes are present in the vicinity of the interface between the first layer 121 and the second layer 122. In addition, a phenomenon in which holes reach the electron-transporting layer 114 without recombining with electrons can be suppressed.

Meanwhile, electrons are injected from the second electrode 103 into the second layer 122 of the light-emitting layer 113 through the electron-transporting layer 114. The electrons injected into the second layer 122 are transported through the second layer 122 and further injected into the first layer 121. Here, the organic compound having a hole-transporting property included in the first layer 121 is a substance which has a hole-transporting property higher than an electron-transporting property, and thus, the electrons injected into the first layer 121 are difficult to move. Consequently, a phenomenon in which electrons reach the hole-transporting layer 112 without recombining with holes can be suppressed.

Accordingly, a large number of holes and electrons are present in a region in the vicinity of the interface between the first layer 121 and the second layer 122 of the light-emitting layer 113, whereby the probability of recombination in the vicinity of the interface can be increased. In other words, the light-emitting region is formed in the vicinity of the center of the light-emitting layer 113. As a result, the phenomenon in which holes reach the electron-transporting layer 114 without recombining with electrons or in which electrons reach the hole-transporting layer 112 without recombining with holes can be suppressed, whereby a reduction in the probability of recombination can be prevented. Thus, a reduction in balance of carriers with time can be prevented, which leads to an increase in reliability.

It is preferred that the organic compound having a hole-transporting property be an organic compound capable of being oxidized and reduced and that the highest occupied molecular orbital level (HOMO level) thereof is greater than or equal to −6.0 eV and less than or equal to −5.0 eV in order to allow holes and electrons to be injected into the first layer 121 of the light-emitting layer 113. In addition, the lowest unoccupied molecular orbital level (LUMO level) of the organic compound having a hole-transporting property is preferred to be greater than or equal to −3.0 eV and less than or equal to −2.0 eV.

As such an organic compound having a hole-transporting property, which can be oxidized and reduced, use of anthracene derivatives is particularly preferred among tricyclic polyacene derivatives, tetracyclic polyacene derivatives, pentacyclic polyacene derivatives, and hexacyclic polyacene derivatives. Thus, specific examples of the organic compound having a hole-transporting property, which is included in the first layer 121 of the light-emitting layer 113, include 9,10-diphenylanthracene (abbreviated to DPAnth), N,N-diphenyl-9-[4-(10-phenyl-9-anthryl)phenyl]-9H-carbazol-3-amine (abbreviated to CzA1PA), 4-(10-phenyl-9-anthryl)triphenylamine (abbreviated to DPhPA), N,9-diphenyl-N-[4-(10-phenyl-9-anthryl)phenyl]-9H-carbazol-3-amine (abbreviated to PCAPA), N,9-diphenyl-N-{4-[4-(10-phenyl-9-anthryl)phenyl]phenyl}-9H-carbazol-3-amine (abbreviated to PCAPBA), and the like.

Similarly, it is preferred that the organic compound having an electron-transporting property be an organic compound capable of being oxidized and reduced and that the HOMO level thereof be greater than or equal to −6.0 eV and less than or equal to −5.0 eV in order to allow holes and electrons to be injected into the second layer 122 of the light-emitting layer 113. In addition, the lowest unoccupied molecular orbital level (LUMO level) of the organic compound having an electron-transporting property is preferred to be greater than or equal to −3.0 eV and less than or equal to −2.0 eV.

As such an organic compound having an electron-transporting property, which can be oxidized and reduced, a tricyclic polyacene derivative, a tetracyclic polyacene derivative, a pentacyclic polyacene derivative, or a hexacyclic polyacene derivative can be used. Specifically, an anthracene derivative, a phenanthrene derivative, a pyrene derivative, a chrysene derivative, a dibenzo[g,p]chrysene derivative, and the like are given. For example, as a compound having an electron-transporting property, which can be used for the second layer 122 of the light-emitting layer 113, 9-[4-(10-phenyl-9-anthryl)phenyl]-9H-carbazole (abbreviated to CzPA), 3,6-diphenyl-9-[4-(10-phenyl-9-anthryl)phenyl]-9H-carbazole (abbreviated to DPCzPA), 9,10-bis(3,5-diphenyl-phenyl)anthracene (abbreviated to DPPA), 9,10-di(2-naphthyl)anthracene (abbreviated to DNA), 2-tert-butyl-9,10-di(2-naphthyl)anthracene (abbreviated to t-BuDNA), 9,9′-bianthryl (abbreviated to BANT), 9,9′-(stilbene-3,3′-diyl)diphenanthrene (abbreviated to DPNS), 9,9′-(stilbene-4,4′-diyl)diphenanthrene (abbreviated to DPNS2), 3,3′,3″-(benzene-1,3,5-triyl)tripyrene (abbreviated to TPB3), and the like can be given.

As described above with reference to FIG. 1B, the light-emitting element in accordance with an embodiment of the present invention is formed so that holes can be injected from the first layer 121 into the second layer 122 of the light-emitting layer 113. Therefore, it is preferred that the difference in HOMO level between the organic compound having a hole-transporting property and the organic compound having an electron-transporting property be small. Also, the light-emitting element is formed so that electrons can be injected from the second layer 122 into the first layer 121 of the light-emitting layer 113. Therefore, it is preferred that the difference in LUMO level between the organic compound having a hole-transporting property and the organic compound having an electron-transporting property be small. When the difference in HOMO level between the organic compound having a hole-transporting property and the organic compound having an electron-transporting property is large, the light-emitting region can be shifted toward either the first layer 121 or the second layer 122. In a similar way, when the difference in LUMO level between the organic compound having a hole-transporting property and the organic compound having an electron-transporting property is large, the light-emitting region can be shifted toward either the first layer 121 or the second layer 122. Therefore, it is preferred that the difference in HOMO level between the organic compound having a hole-transporting property and the organic compound having an electron-transporting property be less than or equal to 0.3 eV, and more preferably, less than or equal to 0.1 eV. Further, it is preferred that the difference in LUMO level between the organic compound having a hole-transporting property and the organic compound having an electron-transporting property be less than or equal to 0.3 eV, and more preferably, less than or equal to 0.1 eV.

Since light is emitted from the light-emitting element by recombination of electrons and holes, it is preferred that the organic compound used for the light-emitting layer 113 be stable with respect to repetitive oxidation and reduction. In other words, it is preferred that the organic compound undergo reversible oxidation and reduction. In particular, the organic compound having a hole-transporting property and the organic compound having an electron-transporting property are preferred to be stable even if the oxidation and reduction thereof are repeated. Stability to repetitive oxidation and reduction can be confirmed by cyclic voltammetry (CV) measurement.

Specifically, changes in values of an oxidation peak potertial (Eₚₐ) in the oxidation or a reduction peak potertial (E_{pc}) in the reduction of the organic compound, changes of the peak shape, and the like are observed, whereby whether or not the organic compounds are stable to the repetitive oxidation and reduction can be confirmed. It is preferred that in the organic compound having a hole-transporting property and the organic compound having an electron-transporting property used for the light-emitting layer 113, the changes in the intensity of the oxidation peak potertial and the intensity of the reduction peak potertial be less than 50%, more preferably, less than 30%. In other words, for example, the peak intensity of 50% or higher is preferred to be kept even when the oxidation peak potertial decreases. More preferably, a peak intensity of 70% or higher is kept. In addition, the changes in the values of the oxidation peak potertial and the reduction peak potertial are preferred to be 0.05 V or lower, more preferably, 0.02 V or lower.

Further, when the first layer 121 and the second layer 122 of the light-emitting layer 113 each include a different substance having a high light-emitting property, there is a possibility that light might be emitted from only one of the layers. However, when the first organic compound included in the first layer 121 and the second organic compound included in the second layer 122 are the same substance, light can be made to be emitted in the vicinity of the center of the light-emitting layer 113. Accordingly, it is preferred that the substance having a high light-emitting property included in the first layer 121 and the substance having a high light-emitting property included in the second layer 122 be any of the anthracene derivatives described in Embodiment 1. Since the anthracene derivatives described in Embodiment 1 have high emission efficiency, application thereof to the structure described in this embodiment leads to the formation of a light-emitting element with high emission efficiency and a long lifetime.

In the light-emitting element described in this embodiment, the light-emitting region is not located in the vicinity of the interface between the light-emitting layer 113 and the hole-transporting layer 112 or the interface between the light-emitting layer 113 and the electron-transporting layer 114, but formed in the vicinity of the center of the light-emitting layer 113. Therefore, there is almost no influence of deterioration which is caused when the light-emitting region is adjacent to the hole-transporting layer 112 or the electron-transporting layer 114. Therefore, a light-emitting element having a long lifetime and negligible deterioration can be obtained. Furthermore, since the light-emitting layer 113 of the light-emitting element in this embodiment includes the compound which is stable in repetitive oxidation and reduction, it is scarcely deteriorated even if light emission caused by recombination of holes and electrons is repeated. Therefore, the above structure is preferred because a light-emitting element having a long lifetime can be obtained.

Since in the light-emitting element described in this embodiment, the organic compound included in the first layer 121 and the organic compound included in the second layer 122 of the light-emitting layer 113 emit light of similar colors, light with high color purity can be obtained even if not only the organic compound included in the first layer 121 but also the organic compound included in the second layer 122 emits light. Further, since each of the anthracene derivatives described in Embodiment 1 is a blue emissive substance having a high light-emitting property, the element structure described in this embodiment is particularly effective for use in a blue emissive light-emitting element and a blue-green emissive light-emitting element. Development of a blue color is the most essential for fabrication of a full-color display, and the deterioration can be suppressed by applying the present invention. This embodiment can be combined with any other embodiment as appropriate.

Moreover, as regards the light-emitting element described in this embodiment, the first layer 121 and the second layer 122 of the light-emitting layer 113 may have a structure in which the anthracene derivative described in Embodiment 1 is dispersed in a host material and may also have a structure in which the anthracene derivative described in Embodiment 1 is not dispersed in a host material but is used solely.

### Embodiment 6

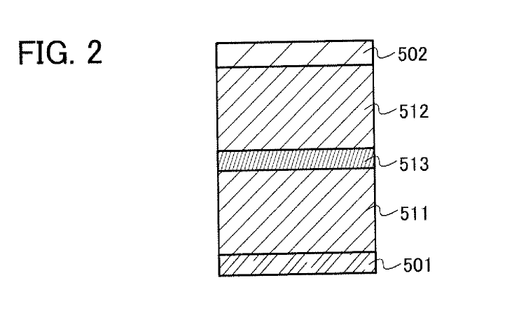

In this embodiment, a mode of a light-emitting element with a structure in which a plurality of light-emitting units in accordance with the above embodiments are stacked (hereinafter referred to as a stacked element) is described with reference to FIG. 2. This light-emitting element is a light-emitting element having a plurality of light-emitting units between a first electrode and a second electrode. Each of the light-emitting units can have a structure similar to that of the layer 102 including an organic compound described in Embodiment 2. That is, the light-emitting element described in Embodiment 2 is a light-emitting element having one light-emitting unit, whereas the light-emitting element described in this embodiment has a plurality of light-emitting units.

In FIG. 2, a first light-emitting unit 511 and a second light-emitting unit 512 are stacked between a first electrode 501 and a second electrode 502. The first electrode 501 and the second electrode 502 may be similar to those described in Embodiment 2. The first light-emitting unit 511 and the second light-emitting unit 512 may have the same structure or different structure, and the structure of the layer including an organic compound described in any of Embodiments 2 to 5 can be applied thereto.

A charge generation layer 513 includes a composite material of an organic compound and a metal oxide. This composite material of an organic compound and metal oxide is described in Embodiment 2 or 5, and includes an organic compound and metal oxide such as vanadium oxide, molybdenum oxide, or tungsten oxide. As the organic compound, various compounds such as an aromatic amine compound, a carbazole derivative, aromatic hydrocarbon, and a high molecular compound (an oligomer, a dendrimer, a polymer, or the like) can be used. An organic compound having a hole-transporting property, which has a hole mobility of greater than or equal to 10⁻⁶ cm²/Vs, is preferred to be applied as the organic compound. Note that a substance other than the above substances may be used as long as it has a hole-transporting property higher than an electron-transporting property. The composite material of an organic compound and metal oxide is superior in carrier injection property and carrier-transporting property, and accordingly, low-voltage driving and low-current driving can be realized.

Note that the charge generation layer 513 may be formed with a combination of a composite material of an organic compound and a metal oxide, and other materials. For example, the charge generation layer 513 may be formed with a combination of a layer including the composite material of an organic compound and a metal oxide and a layer including one compound selected from electron-donating substances and a compound having a high electron-transporting property. Further, the charge generation layer 513 may be formed with a combination of a layer including the composite material of an organic compound and a metal oxide and a transparent conductive film.

In any case, the charge generation layer 513 interposed between the first light-emitting unit 511 and the second light-emitting unit 512 is acceptable as long as electrons are injected to one light-emitting unit and holes are injected to the other light-emitting unit when voltage is applied between the first electrode 501 and the second electrode 502. For example, in FIG. 2, any layer can be employed as the charge generation layer 513 as long as the layer injects electrons into the first light-emitting unit 511 and holes into the second light-emitting unit 512 when voltage is applied so that the potertial of the first electrode 501 is higher than that of the second electrode 502.

In this embodiment, a light-emitting element having two light-emitting units is explained; however, this embodiment can be similarly applied to a light-emitting element in which three or more light-emitting units are stacked. When the charge generation layer is provided between the pair of electrodes so as to partition the plural light-emitting units like the light-emitting element of this embodiment, it is possible to provide a light-emitting element which has a long lifetime and is able to emit light at a high luminance at a low current density.

If the light-emitting units are allowed to emit light of different colors from each other, light emission of a desired color can be obtained from the whole light-emitting element. For example, in the case of a light-emitting element having two light-emitting units, if the emission colors of the first light-emitting unit and the second light-emitting unit are made complementary to each other, white light can be obtained from the whole light-emitting element. Note that “complementary color” means a relation between colors which becomes an achromatic color when they are mixed. That is, white light emission can be obtained by mixing light from substances whose emission colors are complementary colors. This technique can be similarly applied to a light-emitting element having three light-emitting units. For example, white light emission can be obtained from the whole light-emitting element when emission colors of the first, second, and third light-emitting units are red, green, and blue, respectively.

This embodiment can be combined with any other embodiment as appropriate.

### Embodiment 7

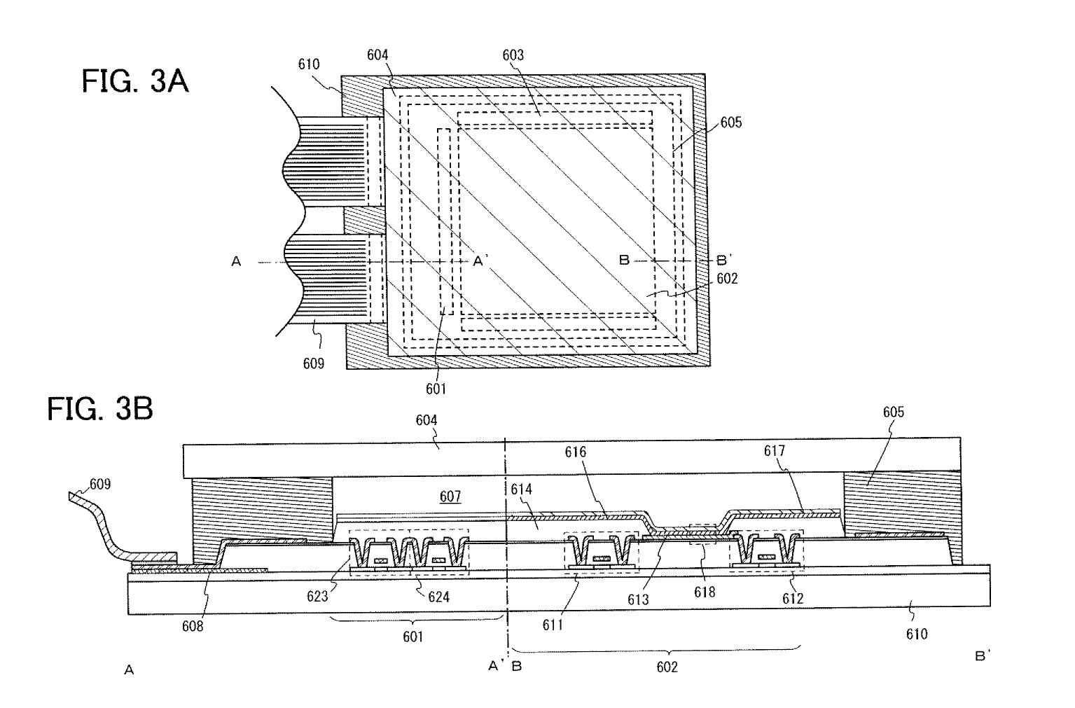

In this embodiment, a light-emitting device manufactured using the anthracene derivative described in Embodiment 1 is described with reference to FIGS. 3A and 3B. FIG. 3A is a top view of the light-emitting device, and FIG. 3B is a cross-sectional view taken along lines A-A′ and B-B′ of FIG. 3A. This light-emitting device includes a driver circuit portion (a source side driver circuit) 601 and a driver circuit portion (a gate side driver circuit) 603, each illustrated in dotted lines, which are configured to control the light emission of the light-emitting element provided in a pixel portion 602. A reference numeral 604 represents a sealing substrate, a reference numeral 605 represents a sealant, and the inside surrounded by the sealant 605 is a space 607.

A lead wiring 608 is a wiring for transmitting signals to be inputted to the source side driver circuit 601 and the gate side driver circuit 603 and receives a video signal, a clock signal, a start signal, a reset signal, and the like from a flexible printed circuit (FPC) 609 which is an external input terminal. Although only the FPC is illustrated here, the FPC may be provided with a printed wiring board (PWB). The light-emitting device described in the specification includes not only a light-emitting device itself but also a state in which an FPC or a PWB is attached thereto.

Next, a sectional structure will be described with reference to FIG. 3B. Although the driver circuit portions and the pixel portion are formed over an element substrate 610, the source side driver circuit 601, which is one of the driver circuit portions, and one pixel in the pixel portion 602 are illustrated here.

Note that as the source side driver circuit 601, a CMOS circuit in which an n-channel TFT 623 and a p-channel TFT 624 are combined is formed. The driver circuit may be formed by various CMOS circuits, PMOS circuits, or NMOS circuits. In this embodiment, although a driver-integrated type structure in which a driver circuit is formed over a substrate is described, a driver circuit is not necessarily formed over the substrate but can be formed externally.

The pixel portion 602 has a plurality of pixels, each of which includes a switching TFT 611, a current control TFT 612, and a first electrode 613 which is electrically connected to a drain of the current control TFT 612. An insulator 614 is formed so as to cover the edge of the first electrode 613. Here, the insulator 614 is formed using a positive photosensitive acrylic resin.

In order to enhance coverage by a subsequently deposited layer, the insulator 614 is provided so that at least one of an upper edge portion and a lower edge portion thereof can have a curved surface with a curvature. For example, in the case of using a positive photosensitive acrylic as a material of the insulator 614, it is preferred that only the upper end portion of the insulator 614 have a curved surface with curvature (a radius of curvature of 0.2 to 3 μm). Note that the insulator 614 can be formed using either a negative type that becomes insoluble in an etchant after photo-irradiation or a positive type that becomes insoluble in an etchant after photo-irradiation.

A layer including an organic compound (EL layer) 616 and a second electrode 617 are formed over the first electrode 613. Here, a material having a high work function is preferred to be used as a material used for the first electrode 613 which serves as an anode. For example, a single-layer film such as an ITO film, an indium tin oxide film containing silicon, an indium oxide film containing 2 to 20 wt % of zinc oxide, a titanium nitride film, a chromium film, a tungsten film, a Zn film, a Pt film, or the like; a stacked film such as a stack of a titanium nitride film and a film containing aluminum as its main component; or a stacked film having a three-layer structure of a titanium nitride film, a film containing aluminum as its main component, and another titanium nitride film can be used. A stacked layer structure is preferred because the resistance as a wiring is low, and a good ohmic contact can be obtained.

The EL layer 616 is formed by various methods such as an evaporation method using an evaporation mask, an ink-jet method, a spin coating method, or the like. The EL layer 616 includes the anthracene derivative described in Embodiment 1. As another material included in the EL layer 616, a low molecular compound or a high molecular compound (including an oligomer or a dendrimer) may be used. Further, not only organic compounds but also inorganic compounds can be used for the material for forming the EL layer 616.

Further, it is preferred that a material with a low work function (e.g., Al, Mg, Li, Ca, or an alloy or a compound thereof such as Mg—Ag, Mg—In, Al—Li, LiF, or CaF₂) be used as a material used for the second electrode 617 which is formed over the EL layer 616 and serves as a cathode. When light generated in the EL layer 616 passes through the second electrode 617, it is preferred that the second electrode 617 be formed by a stack of a thin metal film and a transparent conductive film (ITO, indium oxide containing 2 to 20 wt % of zinc oxide, indium oxide-tin oxide containing silicon or silicon oxide, zinc oxide, or the like).

The sealing substrate 604 is attached to the element substrate 610 with the sealant 605, whereby a light-emitting element 618 is provided in the space 607 surrounded by the element substrate 610, the sealing substrate 604, and the sealant 605. The space 607 is filled with a filler, which may be an inert gas (e.g., nitrogen or argon) or the sealant 605.

As a material for the sealant 605, it is preferred that an epoxy resin be used. In addition, it is preferred that the material allow permeation of moisture or oxygen as little as possible. As the sealing substrate 604, a plastic substrate formed of fiberglass-reinforced plastics (FRP), polyvinyl fluoride (PVF), a polyester, an acrylic, or the like can be used besides a glass substrate or a quartz substrate.

In this manner, the light-emitting device manufactured using the anthracene derivative described in Embodiment 1 can be obtained.

Since the anthracene derivative described in Embodiment 1 is used for the light-emitting device of this embodiment, the light-emitting device with favorable characteristics can be obtained. Specifically, a light-emitting device having a long lifetime can be obtained.

Also, since the anthracene derivatives described in Embodiment 1 have high emission efficiency, a light-emitting device with low power consumption can be provided.

Further, since the light-emitting element in which any of the anthracene derivatives described in Embodiment 1 is used can emit blue light with high color purity at high efficiency, the light-emitting element are suitable for use in full-color displays. Further, since the light-emitting element can emit blue light with low power consumption and has a long lifetime, the light-emitting element is suitable for use in full-color displays.

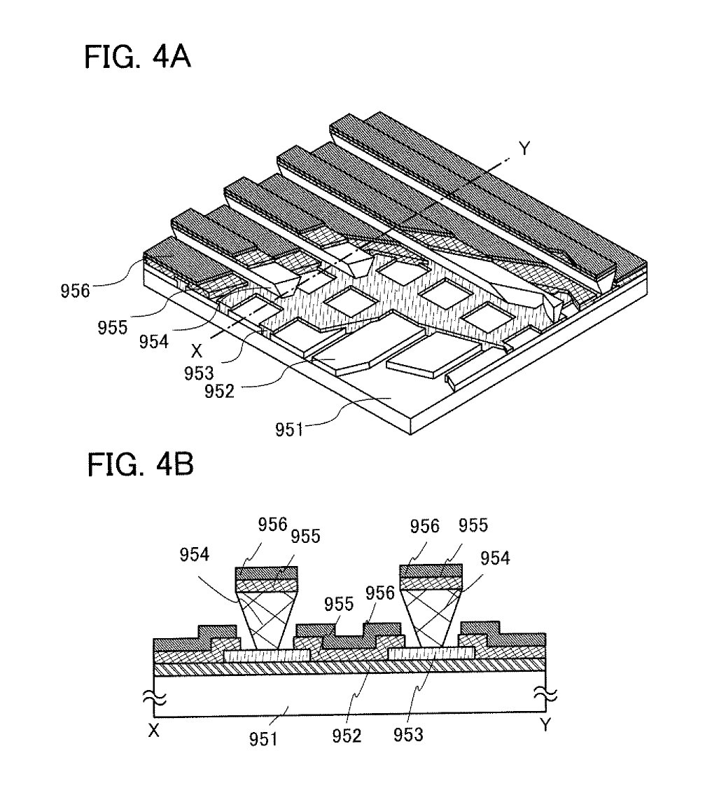

As described above, in this embodiment, an active matrix type light-emitting device in which operation of a light-emitting element is controlled by a transistor is described. Alternatively, a passive matrix type light-emitting device may also be used. FIGS. 4A and 4B illustrate a passive matrix type light-emitting device manufactured by applying an embodiment of the present invention. FIG. 4A is a perspective view of the light-emitting device, and FIG. 4B is a cross-sectional view taken along line X-Y of FIG. 4A. As illustrated in FIGS. 4A and 413, an electrode 952 and an electrode 956 are provided over a substrate 951, and an EL layer 955 is provided between the electrode 952 and the electrode 956. The edge of the electrode 952 is covered with an insulating layer 953. A partition layer 954 is provided on the insulating layer 953. The sidewalk of the partition layer 954 are aslope so that a distance between both sidewalls is gradually narrowed toward the surface of the substrate 951. That is, a cross section in the direction of a narrow side of the partition layer 954 has a trapezoidal shape, and a lower side (which is in the same direction as a surface of the insulating layer 953 and is in contact with the insulating layer 953) is shorter than an upper side (which is in the same direction as the surface of the insulating layer 953 and is not in contact with the insulating layer 953). By providing the partition layer 954 in this manner, defects of the light-emitting element due to a cross talk and the like can be prevented. By using the above-described light-emitting element, a passive matrix type light-emitting device having a long lifetime can be obtained. Further, a light-emitting device with low power consumption can be manufactured.

### Embodiment 8

In this embodiment, an electronic device in accordance with an embodiment of the present invention which includes the light-emitting device described in Embodiment 7 in part thereof is described. The electronic device in accordance with an embodiment of the present invention includes the anthracene derivative described in Embodiment 1, and has a display portion with a long lifetime. Further, it has a display portion with low power consumption.

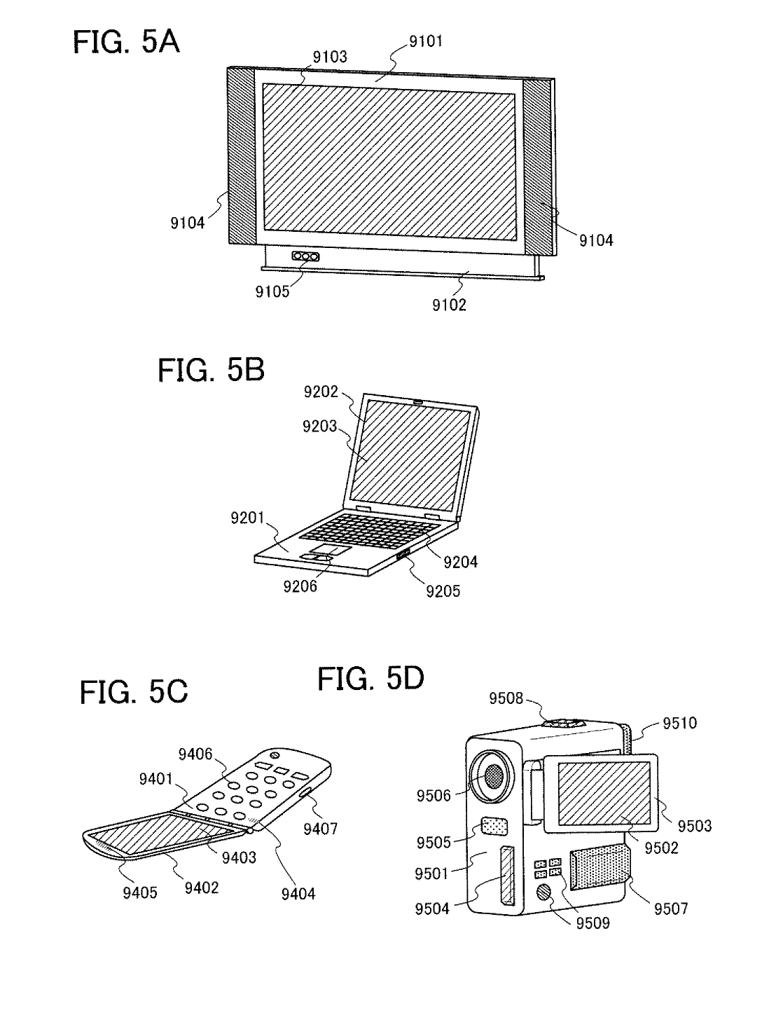

Examples of electronic devices each including a light-emitting element manufactured using the anthracene derivative described in Embodiment 1 include video cameras, digital cameras, goggle type displays, navigation systems, audio playback devices (e.g., car audio systems and other audio systems), computers, game machines, portable information terminals (e.g., mobile computers, mobile phones, portable game machines, and electronic books), image playback devices provided with recording media (i.e., devices that are capable of playing back recording media such as digital versatile discs (DVDs) and equipped with display devices that can display the image), and the like. Specific examples of such electronic devices are illustrated in FIGS. 5A to 5D.

FIG. 5A illustrates a television set in accordance with an embodiment of the present invention, which includes a housing 9101, a supporting base 9102, a display portion 9103, speaker portions 9104, a video input terminal 9105, and the like. In the display portion 9103 of this television set, light-emitting elements similar to those described in any of Embodiments 2 to 6 are arranged in a matrix form. The features of the light-emitting element are exemplified by high emission efficiency and a long lifetime. The display portion 9103 which includes the light-emitting elements has a similar feature. Therefore, in the television device, image quality is hardly deteriorated and low power consumption is attained. With such a feature, a deterioration compensation functional circuit and a power supply circuit can be significantly reduced or downsized in the television set; therefore, the housing 9101 and the supporting base 9102 can be lightened and downsized. Since the television set in accordance with an embodiment of the present invention can realize low power consumption, high image quality, and reduction in size and weight, products suitable for any residential environment can be provided. Also, since a light-emitting element utilizing any of the anthracene derivatives described in Embodiment 1 can emit blue light with high color purity, a television set that can display full-color image and possesses a display portion having a long lifetime can be obtained.

FIG. 5B illustrates a computer in accordance with an embodiment of the present invention, which includes a main body 9201, a housing 9202, a display portion 9203, a keyboard 9204, an external connection port 9205, a pointing device 9206, and the like. In the display portion 9203 of this computer, light-emitting elements similar to those described in any of Embodiments 2 to 6 are arranged in a matrix form. The features of the light-emitting element are exemplified by high emission efficiency and a long lifetime. The display portion 9203 which includes the light-emitting elements has a similar feature. Therefore, in the computer, image quality is hardly deteriorated and low power consumption is attained. With such a feature, a deterioration compensation functional circuit and a power supply circuit can be significantly reduced or downsized in the computer; therefore, the main body 9201 and the housing 9202 can be lightened and downsized. Since the computer in accordance with an embodiment of the present invention can realize low power consumption, high image quality and reduction in size and weight, products suitable for environment can be provided. Also, since a light-emitting element utilizing any of the anthracene derivatives described in Embodiment 1 can emit blue light with high color purity, a computer that can display full-color image and possesses a display portion having a long lifetime can be obtained.

FIG. 5C illustrates a mobile phone in accordance with an embodiment of the present invention, which includes a main body 9401, a housing 9402, a display portion 9403, an audio input portion 9404, an audio output portion 9405, an operation key 9406, an external connecting port 9407, and the like. In the display portion 9403 of this mobile phone, light-emitting elements similar to those described in any of Embodiments 2 to 6 are arranged in a matrix form. The features of the light-emitting element are exemplified by high emission efficiency and a long lifetime. The display portion 9403 which includes the light-emitting elements has a similar feature. Therefore, in this mobile phone, image quality is hardly deteriorated and low power consumption is attained. With such a feature, a deterioration compensation functional circuit and a power supply circuit can be significantly reduced or downsized in the mobile phone; therefore, the main body 9401 and the housing 9402 can be lightened and downsized. Since the mobile phone in accordance with an embodiment of the present invention can realize low power consumption, high image quality and reduction in size and weight, products suitable for carrying can be provided. Also, since a light-emitting element utilizing any of the anthracene derivatives described in Embodiment 1 can emit blue light with high color purity, a mobile phone which can display full-color image and possesses a display portion having a long lifetime can be obtained.

FIG. 5D illustrates a camera in accordance with an embodiment of the present invention, which includes a main body 9501, a display portion 9502, a housing 9503, an external connecting port 9504, a remote controller receiving portion 9505, an image receiving portion 9506, a battery 9507, an audio input portion 9508, operation keys 9509, an eye piece portion 9510, and the like. In the display portion 9502 of this camera, light-emitting elements similar to those described in any of Embodiments 2 to 6 are arranged in a matrix form. The features of the light-emitting element are exemplified by high emission efficiency and a long lifetime. The display portion 9502 which includes the light-emitting elements has a similar feature. Therefore, in the camera, image quality is hardly deteriorated and low power consumption is attained. With such a feature, a deterioration compensation functional circuit and a power supply circuit can be significantly reduced or downsized in the camera; therefore, the main body 9501 can be lightened and downsized. Since the camera in accordance with an embodiment of the present invention can realize low power consumption, high image quality and reduction in size and weight, products suitable for carrying can be provided. Also, since a light-emitting element utilizing any of the anthracene derivatives described in Embodiment 1 can emit blue light with high color purity, a camera that can display full-color image and possesses a display portion having a long lifetime can be obtained.

As described above, the range of application of a light-emitting device in accordance with an embodiment of the present invention is extremely wide, and the light-emitting device can be applied to electronic devices in all kinds of fields. By using the anthracene derivative described in Embodiment 1, electronic devices which have display portions with a long lifetime can be provided. Furthermore, with use of the anthracene derivatives described in Embodiment 1, an electronic device which has a display portion with low power consumption can be obtained.

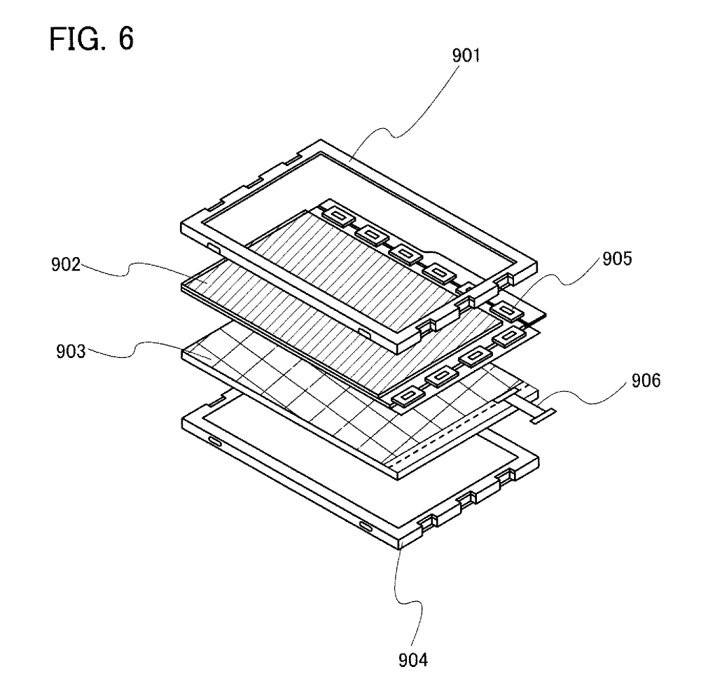

In addition, the light-emitting device in accordance with an embodiment of the present invention can also be used as a lighting device. One mode using the light-emitting device in accordance with an embodiment of the present invention as the lighting device will be described with reference to FIG. 6.

FIG. 6 illustrates an example of a liquid crystal display device using the light-emitting device in accordance with an embodiment of the present invention as a backlight. The liquid crystal display device illustrated in FIG. 6 includes a housing 901, a liquid crystal layer 902, a backlight 903 and a housing 904, and the liquid crystal layer 902 is connected to a driver IC 905. The light-emitting device in accordance with an embodiment of the present invention is used as the backlight 903, and current is supplied through a terminal 906.

By using the light-emitting device in accordance with an embodiment of the present invention as the backlight of the liquid crystal display device, a backlight with reduced power consumption and high emission efficiency can be obtained. The light-emitting device in accordance with an embodiment of the present invention is a lighting device with plane emission, and can have a large area. Therefore, it can readily increase the area of the backlight, which contributes to the increase in the area of the liquid crystal display device. Further, the light-emitting device in accordance with an embodiment of the present invention has a thin shape and exhibits low power consumption; therefore, a display device with low power consumption can be formed in a thin shape. Since the light-emitting device in accordance with an embodiment of the present invention has a long lifetime, a liquid crystal display device using the light-emitting device in accordance with an embodiment of the present invention also has a long lifetime.

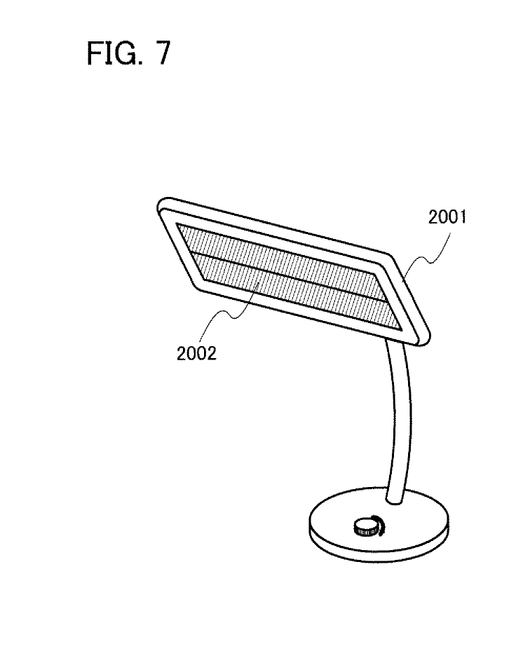

FIG. 7 illustrates an example in which the light-emitting device to which an embodiment of the present invention is applied is used as a desk lamp which is a lighting device. The desk lamp illustrated in FIG. 7 includes a housing 2001 and a light source 2002. The light-emitting device in accordance with an embodiment of the present invention is used as the light source 2002. The light-emitting device in accordance with an embodiment of the present invention has high emission efficiency and has a long lifetime; therefore, a desk lamp also has high emission efficiency and a long lifetime.

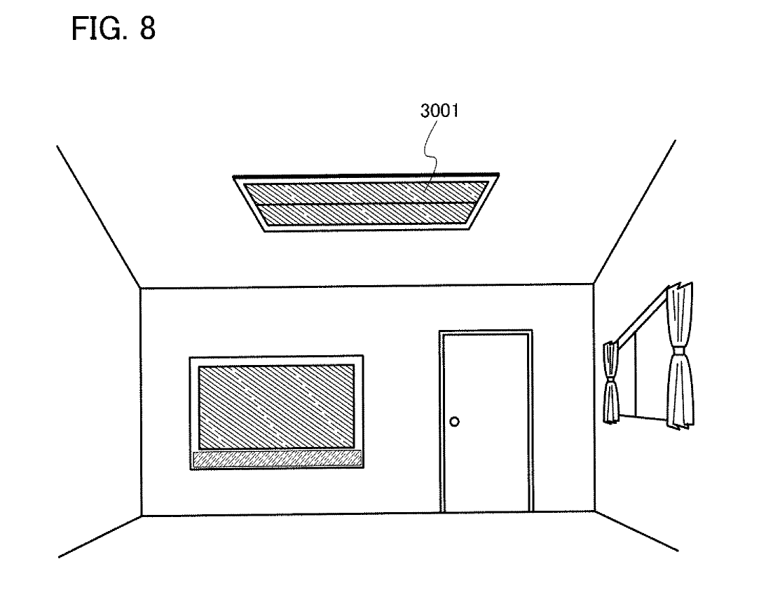

FIG. 8 illustrates an example in which the light-emitting device to which an embodiment of the present invention is applied is used for an indoor lighting device 3001. Since the light-emitting device in accordance with an embodiment of the present invention can also have a large area, it can be used as a lighting device having a large area. Further, the light-emitting device in accordance with an embodiment of the present invention has a thin shape and exhibits low power consumption; accordingly, it can be used as a lighting device having a thin shape and low power consumption.

### Example 1

In this example, the synthetic methods of N-[4-(1-naphthyl)phenyl-N-[4-(10-phenyl-9-anthryl)phenyl]-9-phenyl-9H-carbazol-3-amine (abbreviated to PCNAPA), which is an anthracene derivative in accordance with an embodiment of the present invention and is represented by Structural Formula (101) is specifically described.

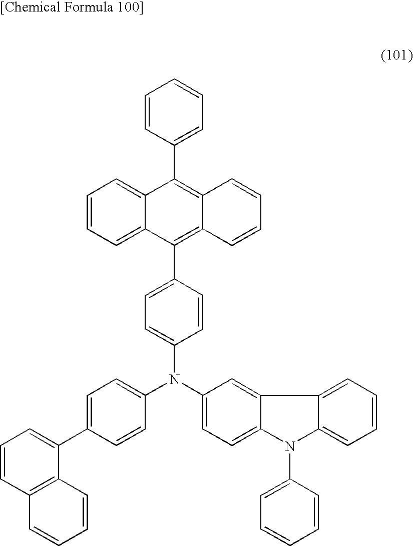

### Step 1: Synthesis of 9-phenylanthracene

Into a 200 mL three-neck flask were put 6.4 g (25 mmol) of 9-bromoanthracene, 3.0 g (25 mmol) of phenylboronic acid, 0.76 g (2.5 mmol) of tri(o-tolyl)phosphine, 60 mL of 1,2-dimethoxyethane (DME), and 25 mL of a 2.0 M potassium carbonate aqueous solution. The mixture was degassed under reduced pressure with stirring, and the atmosphere in the flask was substituted by nitrogen. To the mixture was added 0.11 g (0.50 mmol) of palladium(H) acetate, and the mixture was stirred under nitrogen at 80° C. for 3 hours. After the stirring, water was added to the mixture, and an aqueous layer was extracted with toluene. The obtained extract and the organic layer were combined, washed with brine, and dried with magnesium sulfate. The mixture was subjected to suction filtration through Celite (produced by Wako Pure Chemical Industries, Ltd., catalog number: 531-16855), Florisil (produced by Wako Pure Chemical Industries, Ltd., catalog number: 540-00135), and alumina, and the obtained filtrate was concentrated to give a solid. The solid was recrystallized with a mixed solvent of toluene and methanol to give 5.8 g of a white powder which was a target substance in a yield of 92%. The synthetic scheme of 9-phenylanthracene is shown in (A-1) below.

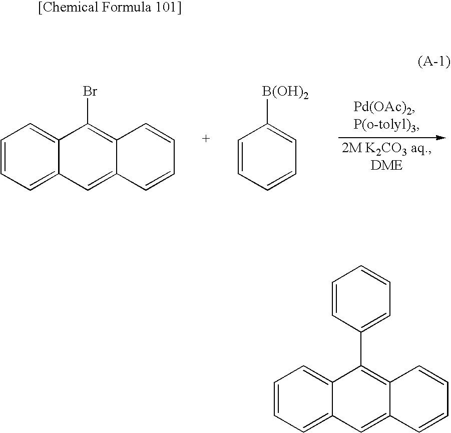

### Step 2: Synthesis of 9-iodo-10-phenylanthracene

Into a 500 mL Erlenmeyer flask was put 4.5 g (18 mmol) of 9-phenylanthracene. To the flask was added 200 mL of acetic acid, followed by heating at 70° C. to dissolve 9-phenylanthracene therein. To the solution was added 5.2 g (13 mmol) of 1,3-diiodo-5,5-dimethylimidazolidine-2,4-dione (DIH), and the solution was stirred under air at 70° C. for 3 hours. After the stirring, about 100 mL of water and about 200 mL of chloroform were added to this solution. The mixture was washed with water twice, and the aqueous layer was extracted with chloroform. The extract was combined with the organic layer and washed with brine, and then the combined organic layer was dried with magnesium sulfate. This mixture was gravity filtered, and the obtained filtrate was concentrated to give a brown solid. This solid was washed with hexane to give 5.8 g of a yellow solid which was a target substance in a yield of 86%. The synthetic scheme is shown in (A-2).

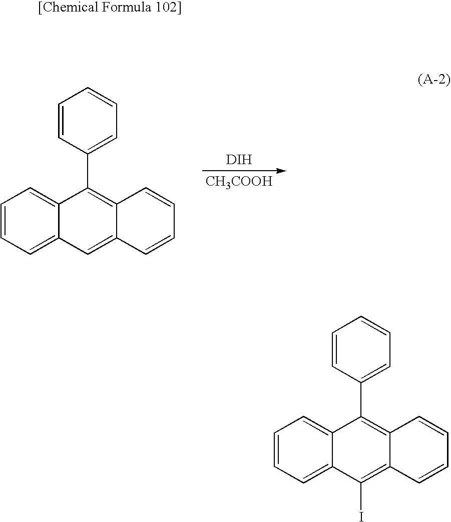

### Step 3: Synthesis of 9-(4-bromophenyl)-10-phenylanthracene

A mixture containing 1.0 g (2.6 mmol) of 9-iodine-10-phenylanthracene, 540 mg (2.7 mmol) of p-bromophenylboronic acid, 46 mg (30 μmol) of tetrakis(triphenylphosphine)palladium(0) (abbreviated to Pd(PPh₃)₄), 3.0 mL (6.0 mmol) of a potassium carbonate aqueous solution (2.0 mol/L), and 10 mL of toluene was stirred at 80° C. for 9 hours. After the stirring, toluene was added to the mixture, and the mixture was filtered through Florisil (produced by Wako Pure Chemical Industries, Ltd., catalog number: 540-00135), Celite (produced by Wako Pure Chemical Industries, Ltd., catalog number: 531-16855), and alumina. The obtained filtrate was washed with water and brine, followed by drying with magnesium sulfate. This mixture was gravity filtered, and the obtained filtrate was concentrated to give a solid, which was subjected to recrystallization with a mixed solvent of chloroform and hexane, resulting in 560 mg of a light brown solid which was a target substance in a yield of 45%. The synthetic scheme of 9-(4-bromophenyl)-10-phenylanthracene is shown in (A-3).

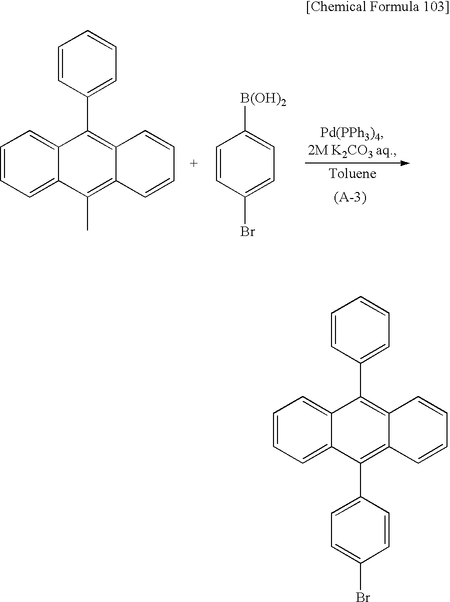

### Step 4: Synthesis of 4-(1-naphthyl)aniline

Into a 500 mL three-neck flask were put 5.0 g (29 mmol) of 4-bromoaniline, 5.0 g (29 mmol) of 1-naphthylboronic acid, and 0.45 g (1.5 mmol) of tris(2-methylphenyl)phosphine, and the atmosphere in the flask was substituted by nitrogen. To this mixture were added 100 mL of toluene, 50 mL of ethanol, and 31 mL of a potassium carbonate aqueous solution (2 mol/L). The mixture was degassed under reduced pressure with stirring. Then, after the mixture was heated at 60° C., 66.2 mg (0.29 mmol) of palladium(II) acetate was added into the mixture. The mixture was refluxed at 80° C. for 2.3 hours. After the reaction, toluene and water were added to the reacted mixture, the organic layer was separated from the aqueous layer, and the aqueous layer was extracted twice with toluene. The extract and the organic layer were combined, washed with brine, and dried with magnesium sulfate. The obtained mixture was gravity filtered to remove magnesium sulfate, and the filtrate was concentrated to give an oily product, which was subjected to suction filtration through Florisil, Celite, and alumina to obtain the filtrate. The obtained filtrate was concentrated to give 2.5 g of an oily substance which was a target substance in a yield of 40%. The synthetic scheme of 4-(1-naphthyl)aniline is shown in (A-5).

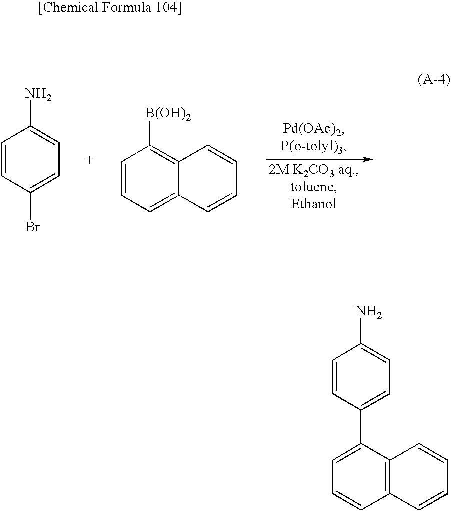

### Step 5: Synthesis of N-[4-(1-naphthyl)phenyl-9-phenyl-9H-carbazol-3-amine (abbreviated to PCNA)

Into a 200 mL three-neck flask were put 3.5 g (11 mmol) of 4-bromo-9-phenyl-9H-carbazole and 3.2 g (33 mmol) of sodium tert-butoxide, and the atmosphere in the flask was substituted by nitrogen. After 2.5 g (11 mmol) of 4-(1-naphthyl)aniline dissolved in 40 mL of toluene was added to this mixture, 15 mL of toluene and 0.5 mL of tri(tert-butyl)phosphine (a 10 wt % hexane solution) were further added to the mixture. The mixture was heated at 60° C., and then 75 mg (0.13 mmol) of bis(dibenzylideneacetone)palladium(0) was added. The mixture was stirred at 80° C. for about 11 hours. After the stirring, 61 mg (0.1 mmol) of bis(dibenzylideneacetone)palladium(0) was added and this mixture was further stirred at 110° C. for about 1 hour. After the stirring, toluene was added to this mixture, and the mixture was subjected to suction filtration through Florisil (produced by Wako Pure Chemical Industries, Ltd., catalog number: 540-00135), Celite (produced by Wako Pure Chemical Industries, Ltd., catalog number: 531-16855), and alumina to obtain filtrate. The obtained filtrate was concentrated to give an oily substance, which was purified by silica gel column chromatography (developing solvent, hexane:toluene=3:2). The obtained fraction was concentrated to obtain 1.4 g of a yellow solid which was a target substance in a yield of 27%. The synthetic scheme of PCNA is shown in (A-5).

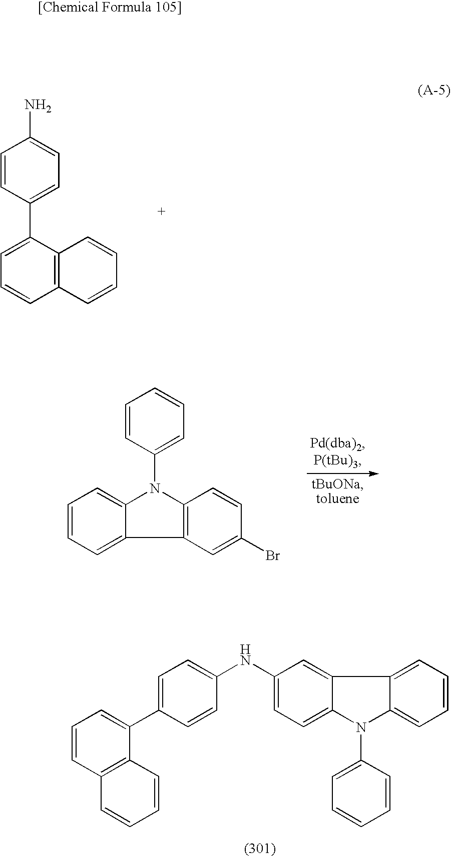

MS spectrum of the solid obtained in the above Step 5 of Example 1 was measured. Measurement result is shown below.

MS (ESI-MS):m/z=461 (M+H)⁺; C₃₄H₂₄N₂ (460.19)

Note that the organic compound and PCNA obtained in this step are novel substances; thus, these substances are included in an embodiment of the present invention.

### Step 6: Synthetic method of N-[4-(1-naphthyl)phenyl-N-[4-(10-phenyl-9-anthryl)phenyl]-9-phenyl-9H-carbazol-3-amine (abbreviation: PCNAPA)

Into a 50 mL three-neck flask were put 0.45 g (1.1 mmol) of 9-(4-bromophenyl)-10-phenylanthracene, 0.3 g (3.2 mmol) of sodium tert-butoxide, and 0.5 g (1.1 mmol) of N-[4-(1-naphthyl)phenyl-9-phenyl-9H-carbazol-3-amine (abbreviated to PCNA)], and the atmosphere in the flask was substituted by nitrogen. Then, 5.4 mL of tri(tert-butyl)phosphine (a 10 wt % hexane solution) was added to this mixture. After the mixture was heated at 60° C., 23 mg (0.04 mmol) of bis(dibenzylideneacetone)palladium(0) was added to the mixture. This mixture was stirred at 80° C. for 4.5 hours. After the stirring, toluene was added to this mixture, and the mixture was subjected to suction filtration through Florisil (produced by Wako Pure Chemical Industries, Ltd., catalog number: 540-00135), Celite (produced by Wako Pure Chemical Industries, Ltd., catalog number: 531-16855), and alumina to obtain filtrate. The obtained filtrate was concentrated to give a solid, which was purified by silica gel column chromatography (developing solvent, hexane:toluene=3:7). The obtained fraction was concentrated to obtain a yellow solid. The obtained solid was subjected to recrystallization with a mixed solvent of toluene and hexane, resulting in 0.2 mg of a yellow solid which was a target substance in a yield of 25%. The synthetic scheme of PCNAPA is shown in (A-6) below.

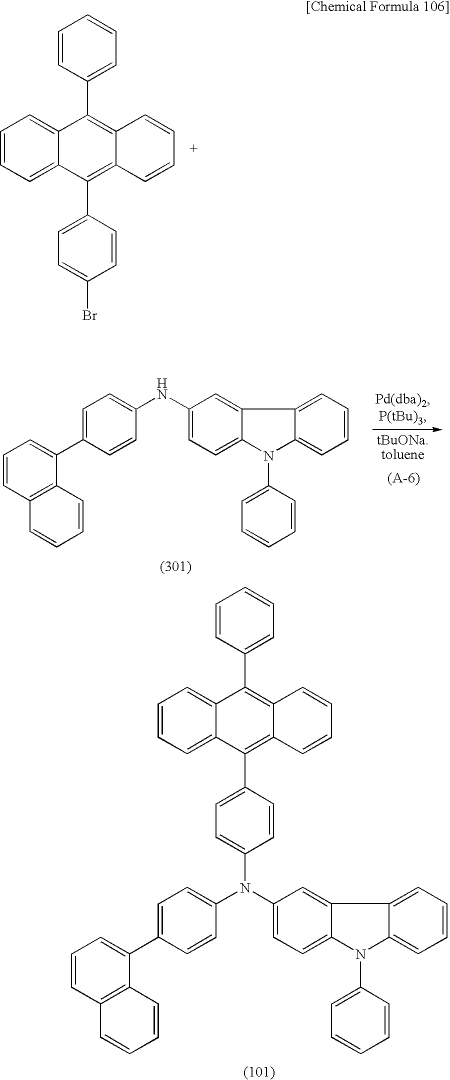

Then, 0.7 g of the obtained yellow solid was sublimated and purified by a train sublimation method. For sublimation purification conditions, the yellow solid was heated at 335° C. under a pressure of 4.6 Pa with a flow rate of argon gas of 5.0 mL/min. After the sublimation purification, 0.6 g of a yellow solid was obtained in a yield of 86%.

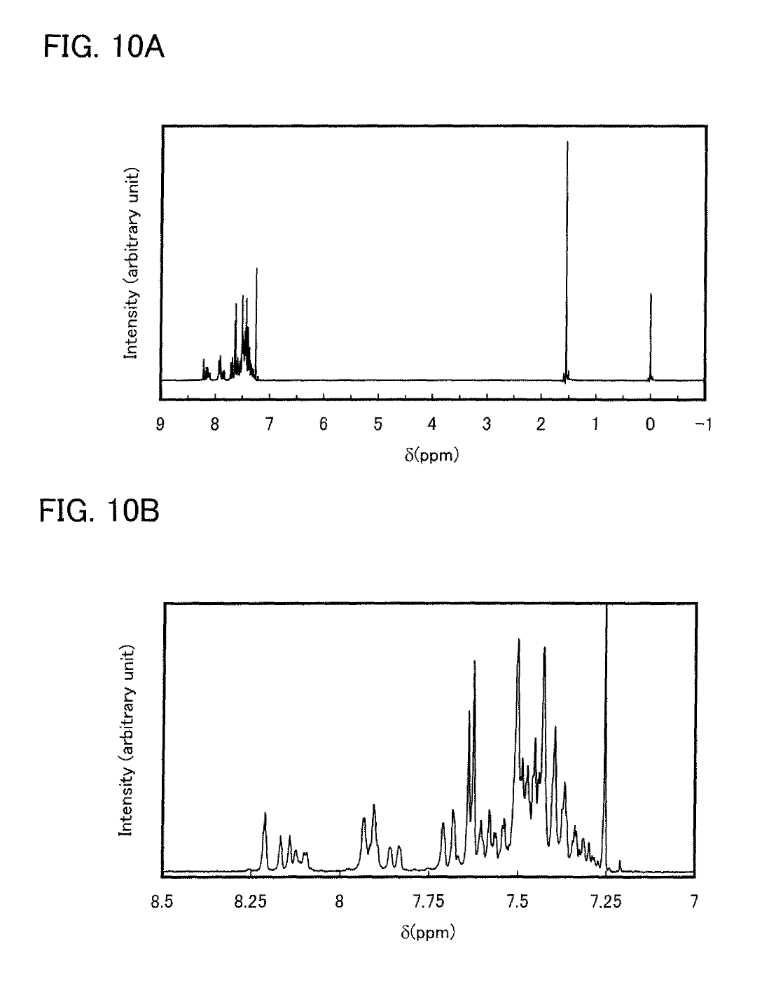

The solid obtained in the above Step 6 was analyzed by ¹H-NMR. The ¹H-NMR chart is shown in FIGS. 10A and 10B. FIG. 10B is a chart in which the range of 7.0 ppm to 8.5 ppm in FIG. 10A is expanded. From the measurement results, it was confirmed that the anthracene derivative PCNAPA which is an embodiment of the present invention and is represented by above Structural Formula (101) was obtained. The measurement data are described below.

¹H-NMR (CDCl₃, 300 MHz): δ=7.27-7.71 (m, 33H), 7.85 (d, J=7.8 Hz, 1H), 7.91-7.94 (m, 3H), 8.09-8.13 (m, 1H), 8.16 (d, J=7.8 Hz, 1H), 8.21 (s, 1H).

The thermogravimetry-differential thermal analysis (TG-DTA) of the obtained PCNAPA was carried out. The measurement was conducted by using a high vacuum differential type differential thermal balance (produced by Bruker AXS K.K., TG-DTA 2410SA). The measurement was carried out under nitrogen stream (flow rate: 200 mL/min) and a normal pressure at a temperature rising rate of 10° C./min. From the relationship between the weight and the temperature (thermogravimetry), it was understood that a 5% weight loss temperature was 500° C. or higher, which is indicative of high thermal stability.

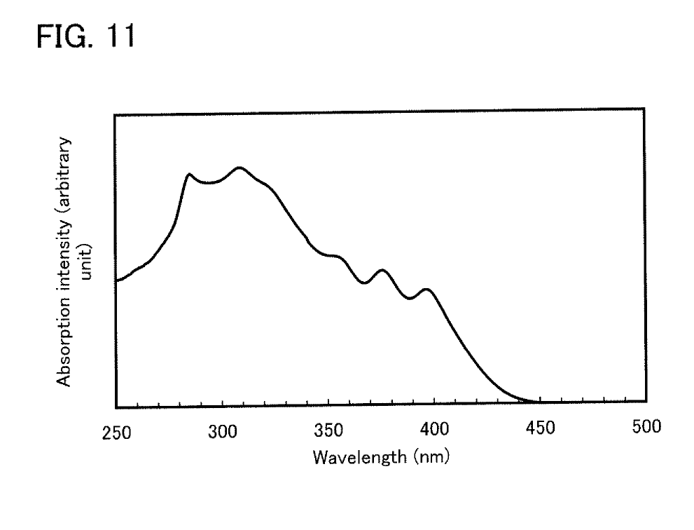

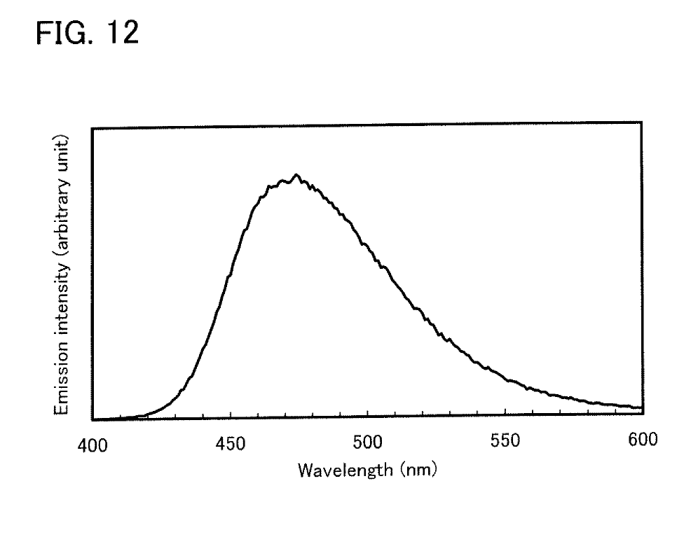

The absorption spectrum and the emission spectrum of a toluene solution of PCNAPA are shown in FIG. 11 and FIG. 12, respectively. An ultraviolet-visible spectrophotometer (produced by JASCO Corporation, V-550) was used for the measurement of the absorption spectrum of the toluene solution in a quartz cell. The absorption spectrum was obtained after subtracting an absorption spectrum of toluene in the quartz cell. In FIG. 11, the horizontal axis indicates the wavelength (nm) and the vertical axis indicates absorption intensity (arbitrary unit). In FIG. 12, the horizontal axis indicates the wavelength (nm) and the vertical axis indicates the light emission intensity (arbitrary unit). Absorption is observed around 307 nm, 353 nm, 375 nm, and 395 nm for the toluene solution. The maximum emission wavelength of the solution was 489 nm (excitation wavelength: 399 nm).

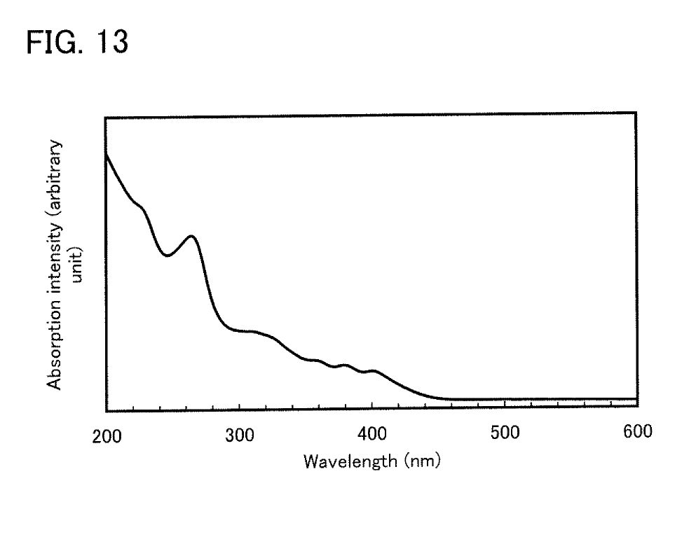

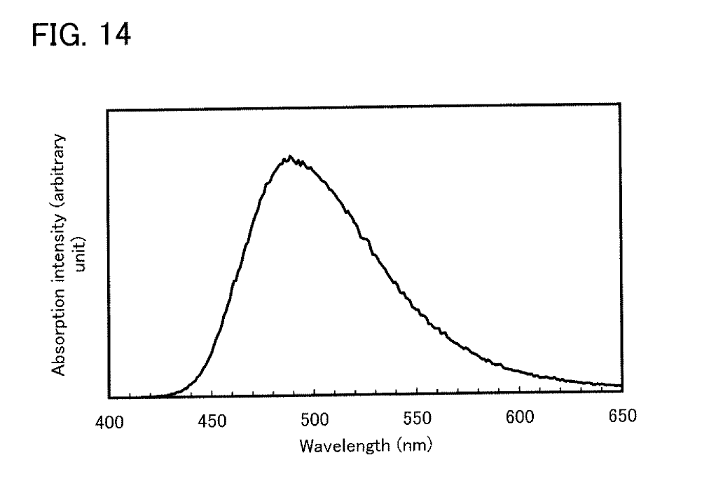

The absorption spectrum and the emission spectrum of a thin film of PCNAPA are shown in FIG. 13 and FIG. 14, respectively. An ultraviolet-visible spectrophotometer (produced by JASCO Corporation, V-550) was used for the measurement of the absorption spectrum. The sample was fabricated by evaporation on a quartz substrate. The absorption spectrum was obtained after subtracting an absorption spectrum of the quartz cell. In FIG. 13, the horizontal axis indicates the wavelength (nm) and the vertical axis indicates absorption intensity (arbitrary unit). In FIG. 14, the horizontal axis indicates the wavelength (nm) and the vertical axis indicates the light emission intensity (arbitrary unit). In the ease of the thin film, absorption was observed around 315 nm, 360 nm, 379 nm, and 400 nm. The maximum emission wavelength was 489 nm (excitation wavelength: 399 nm) in the case of the thin film.

As described above, the measurements reveal that the anthracene derivative PCNAPA, which is an embodiment of the present invention and is represented by Structural Formula (101), exhibits blue light emission in the toluene solution and light blue emission in the thin film state.

The oxidation characteristics and reduction characteristics of PCNAPA were measured. The oxidation characteristics and reduction characteristics were evaluated by cyclic voltammetry (CV) measurement. An electrochemical analyzer (produced by BAS Inc., ALS model 600A) was used for the measurement.

The solution for the CV measurement was prepared by using dehydrated N,N-dimethylformamide (DMF) (produced by Sigma-Aldrich Corp., 99.8%, catalog number: 22705-6) as a solvent, dissolving a supporting electrolyte of tetra-n-butylammonium perchlorate (n-Bu₄NClO₄) (produced by Tokyo Chemical Industry Co., Ltd., catalog number: T0836) at a concentration of 100 mmol/L, and dissolving the sample at a concentration of 1 mmol/L. A platinum electrode (produced by BAS Inc., a PTE platinum electrode) was used as a work electrode, a platinum electrode (produced by BAS Inc., a VC-3 Pt counter electrode (5 cm)) was used as an auxiliary electrode, and an Ag/Ag⁺ electrode (produced by BAS Inc., an RE5 non-aqueous solvent reference electrode) was used as a reference electrode. The measurement was carried out at room temperature. The scan speed at these CV measurements was set at 0.1 V/s.

The reduction reaction characteristics of PCNAPA were evaluated by 100 measurement cycles where the potertial of the work electrode with respect to the reference electrode was scanned from −1.24 V to −2.40 V and then scanned from −2.40 V to −1.24 V in each of the cycles. Similarly, the oxidation characteristics were evaluated by 100 measurement cycles where the potertial was scanned from 0.27 V to 0.55 V and then scanned from 0.55 V to 0.27 V in each of the cycles.

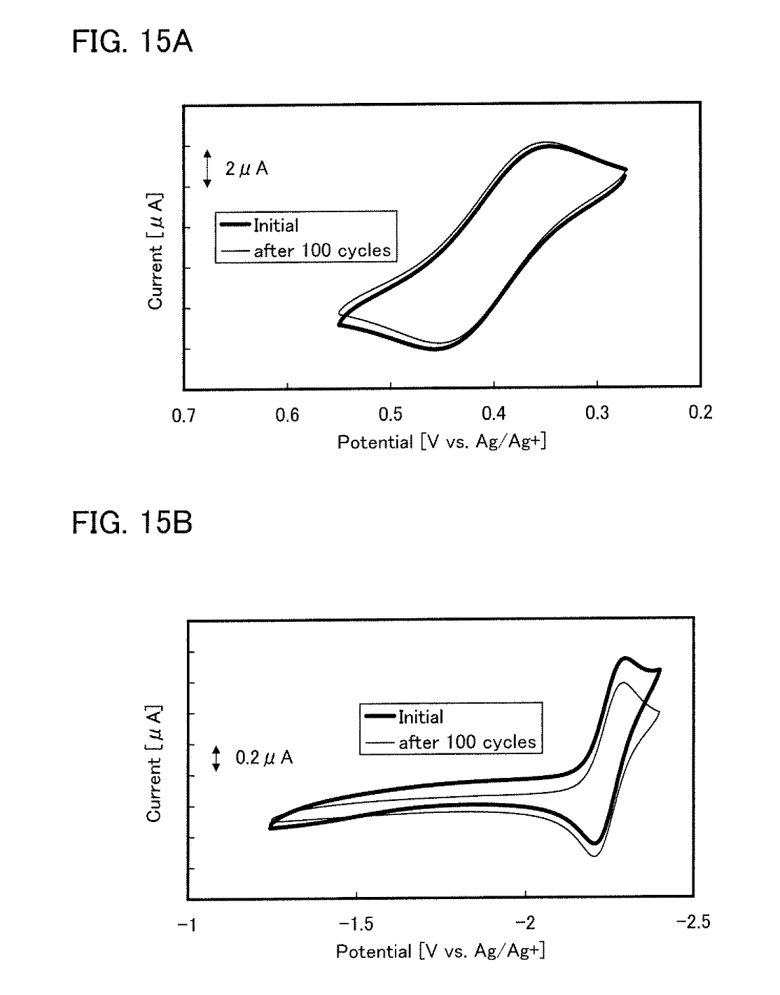

FIGS. 15A and 15B show CV measurement results on the oxidation characteristics and reduction characteristics of PCNAPA, respectively. In each of FIGS. 15A and 15B, the horizontal axis indicates a potertial (V) of the work electrode with respect to the reference electrode, and the vertical axis indicates a current value (μA) flowing between the work electrode and the auxiliary electrode.

It was observed from FIG. 15A that the current indicating the oxidation was observed around 0.40 V (vs. Ag/Ag⁺) and from FIG. 15B that the current indicating the reduction was observed around −2.25 V (vs. Ag/Ag⁺).

Although the scan was repeated as many as 100 cycles, PCNAPA showed no significant change in the peak position of the CV curves in the oxidation and the reduction. The peak intensity thereof remained 95% of the initial state on the oxidation side and 83% of the initial state on the reduction side. Thus, it was confirmed that PCNAPA is relatively stable even when an oxidation from a neutral state to an oxidized state and a reduction from the oxidized state to the neutral state are repeated and when a reduction from the neutral state to a reduced state and an oxidation from the reduced state to the neutral state are repeated.

The results of measuring the thin film of PCNAPA by a photoelectron spectrometer (produced by Riken Keiki Co., Ltd., AC-2) under the air indicated that the HOMO level of PCNAPA was −5.33 eV. The absorption edge was obtained from Tauc plot, with an assumption of direct transition, using data on the absorption spectrum in FIG. 13, and the energy gap thereof was estimated to be 2.89 eV assuming that the absorption edge corresponds to the optical energy gap. The LUMO level was found to be −2.44 eV by calculation from the value of the obtained energy gap and the HOMO level. As thus described, it was found that PCNAPA has a large energy gap of 2.89 eV.

### Example 2

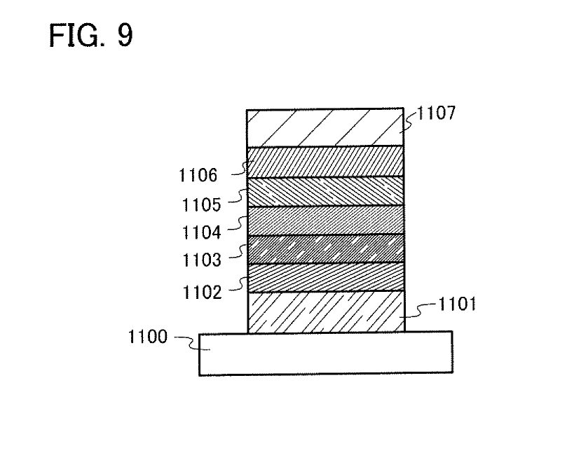

In this example, a light-emitting element in accordance with an embodiment of the present invention is described with reference to FIG. 9. A chemical formula of materials used in this example is given below.

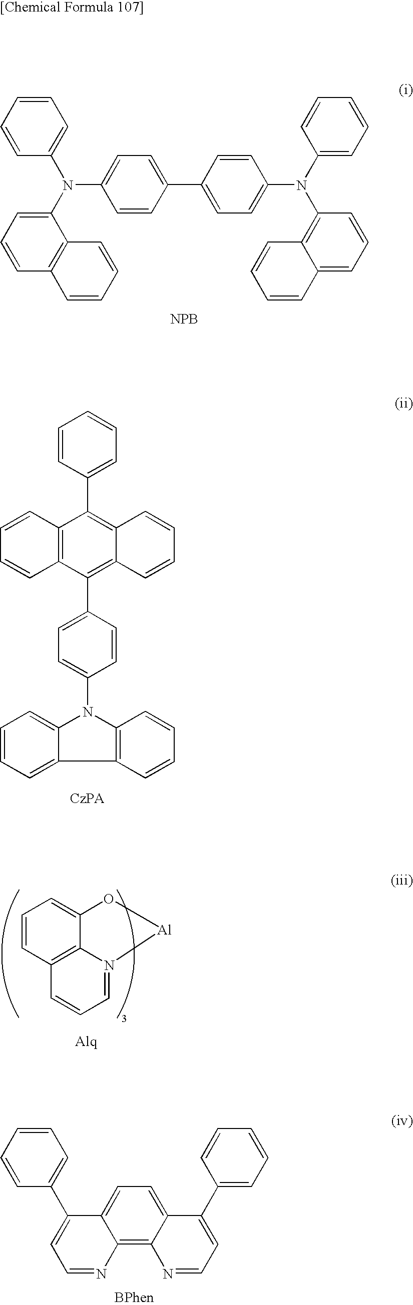

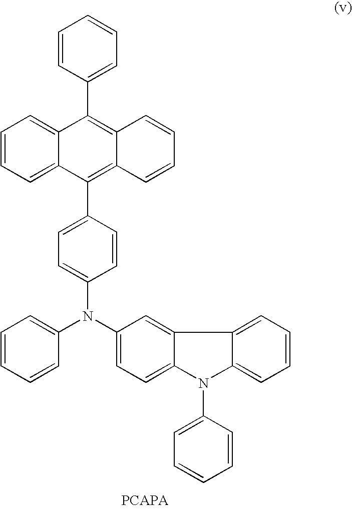

### (Light Emitting Element 1)

The structure of the light-emitting element 1 is explained with reference to FIG. 9. First, indium tin oxide containing silicon oxide was deposited over a substrate 1100 by a sputtering method to form a first electrode 1101. The thickness and the area of the first electrode 1101 were set to 110 nm and 2 mm×2 mm, respectively.

Next, the substrate 1100 over which the first electrode 1101 was formed was fixed to a substrate holder provided in a vacuum evaporation apparatus in such a way that a surface of the substrate 1100, over which the first electrode 1101 was formed, faced downward, and then the pressure was reduced to about 10⁻⁴ Pa. Then, 4,4′-bis[N-(1-naphthyl)-N-phenylamino]biphenyl (abbreviated to NPB) and molybdenum(VI) oxide were co-evaporated over the first electrode 1101, whereby a layer 1102 including a composite material of an organic compound and an inorganic compound was formed. The film thickness of the layer 1102 was set to 50 nm, and the weight ratio between NPB and molybdenum oxide (=NPB:molybdenum oxide) was adjusted to 4:1. Note that the “co-evaporation method” means an evaporation method in which evaporation of a plurality of materials is performed from a plurality of evaporation sources at the same time in one treatment chamber.

Next, NPB was deposited to a thickness of 10 nm over the layer 1102 including the composite material by the evaporation method utilizing resistive heating, whereby a hole-transporting layer 1103 was formed.

Further, by co-evaporation of 9-[4-(10-phenyl-9-anthryl)phenyl]-9H-carbazole (abbreviated to CzPA) and PCNAPA, a light-emitting layer 1104 was formed over the hole-transporting layer 1103 to a thickness of 30 nm. The weight ratio of CzPA and PCNAPA was adjusted to 1:0.10 (=CzPA:PCNAPA).

Then, tris(8-quinolinolato)aluminum(III) (abbreviated to Alq) was deposited over the light-emitting layer 1104 to a thickness of 30 nm by the evaporation method utilizing resistive heating to form an electron-transporting layer 1105.

Furthermore, lithium fluoride was deposited over the electron-transporting layer 1105 to a thickness of 1 nm, whereby an electron injection layer 1106 was formed.

Lastly, aluminum was deposited to a thickness of 200 nm over the electron injection layer 1106 by the evaporation method utilizing resistive heating to form a second electrode 1107. Thus, the light-emitting element 1 was fabricated.

### (Comparative Light-Emitting Element 1)

Next, a comparative light-emitting element 1 was prepared for comparison with the light-emitting element 1. The structure of the comparative light-emitting element 1 is described with reference to FIG. 9. The comparative light-emitting element 1 was prepared using N-phenyl-N-[4-(10-phenyl-9-anthryl)phenyl]-9H-carbazol-3-amine (abbreviated to PCAPA) instead of PCNAPA, which is the anthracene derivative in accordance with an embodiment of the present invention, to form the light-emitting layer 1104. The weight ratio of CzPA and PCAPA was adjusted to 1:0.10 (=CzPA:PCAPA). The structure of the comparative light-emitting element 1 is the same as that of the light-emitting element 1 with the exception of the light-emitting layer 1104.

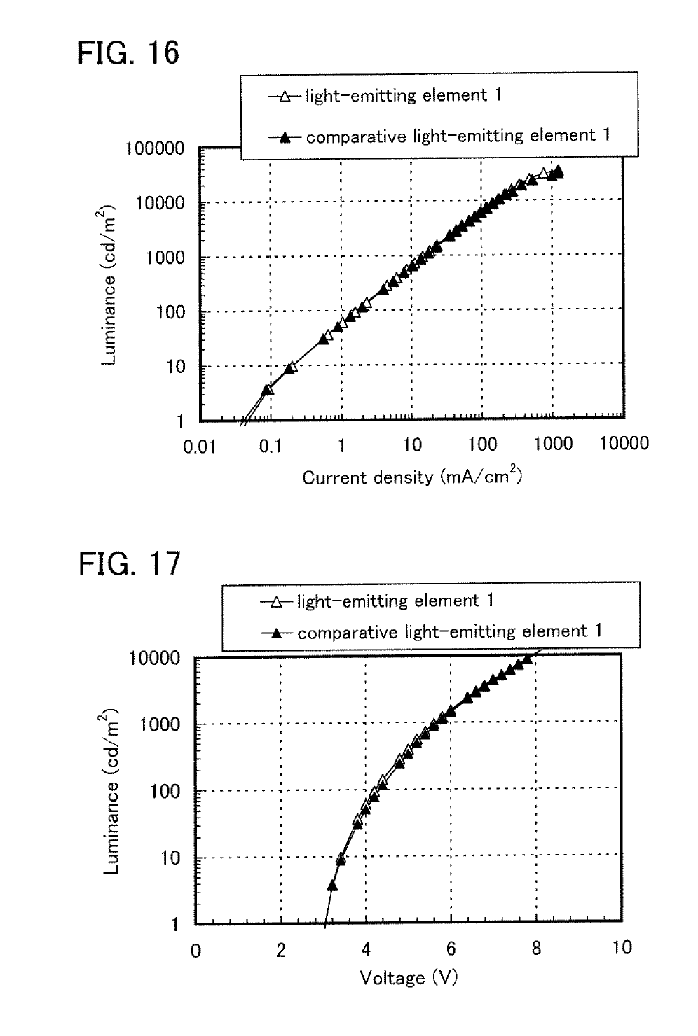

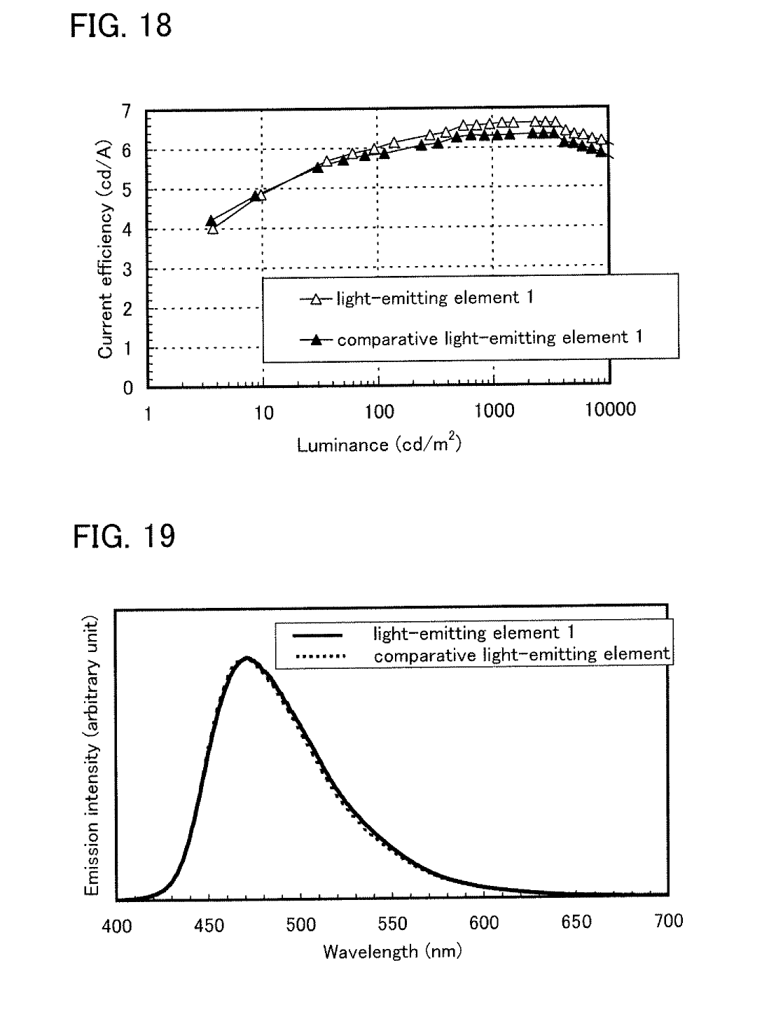

FIG. 16 shows current density-luminance characteristics of the light-emitting element 1 and the comparative light-emitting element 1. FIG. 17 shows voltage-luminance characteristics thereof. FIG. 18 shows luminance-current efficiency characteristics thereof. FIG. 19 shows emission spectra thereof measured at a current of 1 mA. FIG. 19 revealed that the light emission of the light-emitting element 1 originated from PCNAPA, while the light emission of the comparative light-emitting element 1 originated from PCAPA. The CIE chromaticity coordinates of the light-emitting element 1 at a luminance of 950 cd/m² are (x, y)=(0.16, 0.25). FIG. 18 revealed that current efficiency of the light-emitting element 1 at a luminance of 950 cd/m² was 6.6 cd/A, which means that the light-emitting element 1 exhibited a high current efficiency. FIG. 17 revealed that the driving voltage at 950 cd/m² was 5.6 V, and power efficiency was 3.7 μm/W. From these results, it was found that a voltage required to obtain a certain luminance was low and power consumption was also low in the case of the light-emitting element 1. On the other hand, the CIE chromaticity coordinates of the comparative light-emitting element 1 at a luminance of 1100 cd/m² are (x, y)=(0.16, 0.14), indicating that excellent blue emission was obtained. FIG. 18 revealed that current efficiency of the comparative light-emitting element 1 at a luminance of 1100 cd/m² was 6.3 cd/A, which means that the comparative light-emitting element 1 exhibited relatively high current efficiency. FIG. 17 revealed that the driving voltage at 1100 cd/m² was 5.8 V, and power efficiency was 3.4 lm/W. From these results, it was found that a voltage required to obtain a certain luminance was low and power consumption was also low in the case of the comparative light-emitting element 1.

Comparison of the light-emitting element 1 with the comparative light-emitting element 1 revealed that the light-emitting element 1 exhibited a higher current efficiency than the comparative light-emitting element 1. The difference in structure of the emission material in the light-emitting layer between the light-emitting element 1 and the comparative light-emitting element 1 is whether a 1-naphthyl group is provided or not at the terminal of the amine skeleton which is included in the anthracene derivative as an emission material. Whether the 1-naphthyl group was provided or not resulted in the difference in emission efficiency between the light-emitting element 1 and the comparative light-emitting element 1. Accordingly, these results revealed that the 1-naphthyl group at the terminal of the amine skeleton of the anthracene derivative, which is an embodiment of the present invention, provided an effect to realize high emission efficiency. Further, it was found that the use of the anthracene derivative, which is an embodiment of the present invention, in a light-emitting element enabled the production of a light-emitting element which can be driven at a low voltage. Moreover, it was confirmed that a light-emitting element which had high efficiency and low power consumption and was able to be driven at a low voltage was able to be provided.

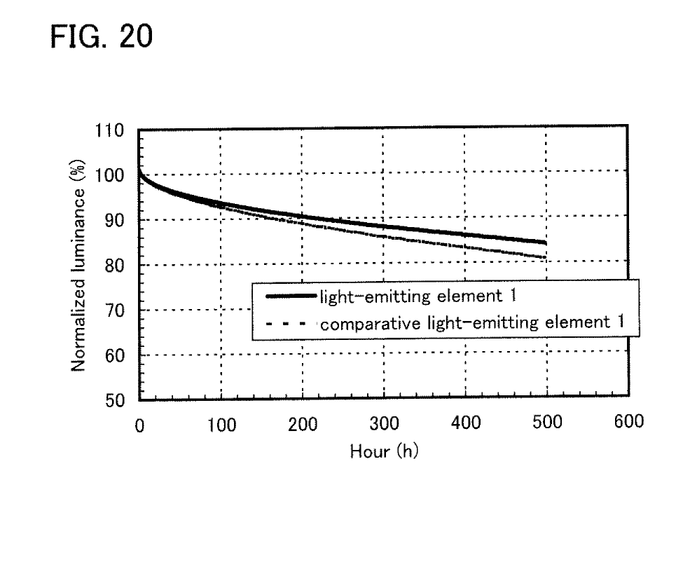

Next, reliability tests of the light-emitting element 1 and the comparative light-emitting element 1 were carried out. Results of the reliability tests are shown in FIG. 20. In FIG. 20, the vertical axis represents normalized luminance (%) on the assumption that an initial luminance is 100%, and the horizontal axis represents driving time (h) of the light-emitting elements. The reliability tests were carried out by driving the light-emitting element 1 and the comparative light-emitting element 1 of this example at a constant current density under the conditions that an initial luminance was set at 1000 cd/m². FIG. 20 revealed that the light-emitting element 1 kept 84% of the initial luminance after the driving for approximately 500 hours; on the other hand, the comparative light-emitting element 1 kept 80% of the initial luminance after the driving for approximately 500 hours. Therefore, it was confirmed that although both the light-emitting element 1 and the comparative light-emitting element 1 exhibited high reliability, the light-emitting element 1 showed higher reliability than the comparative light-emitting element 1. Thus, it was found that the use of the anthracene derivative, which is an embodiment of the present invention, allowed the production of a light-emitting element with a long lifetime. Furthermore, the results of the reliability tests revealed that the 1-naphthyl group at the amine skeleton of the anthracene derivative, which is an embodiment of the present invention, had an effect to realize a light-emitting element with a long lifetime.

The present application is based on Japanese Patert Application serial No. 2008-323612 filed with Japan Patert Office on Dec. 19, 2008, the entire conterts of which are hereby incorporated by reference.

What is claimed is:

1. An anthracene derivative represented by following General Formula (G1). 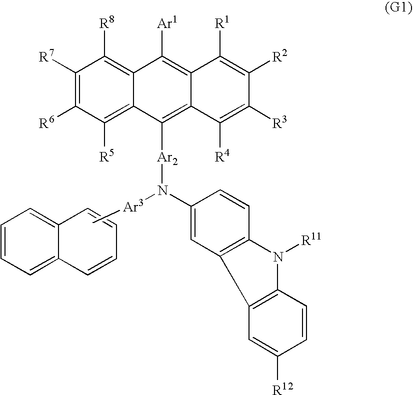
(In General Formula (G1), Ar¹ represents a substituted or unsubstituted aryl group having 6 to 13 carbon atoms; Ar² and Ar³ each independently represent a substituted or unsubstituted arylene group having 6 to 13 carbon atoms; R¹ to R⁸ each represent either hydrogen or an alkyl group having 1 to 4 carbon atoms; R¹¹ represents either an alkyl group having 1 to 4 carbon atoms or a substituted or unsubstituted aryl group having 6 to 13 carbon atoms; and R¹² represents hydrogen, an alkyl group having 1 to 4 carbon atoms, or a substituted or unsubstituted aryl group having 6 to 13 carbon atoms.)

2. An anthracene derivative represented by following General Formula (G1-1). 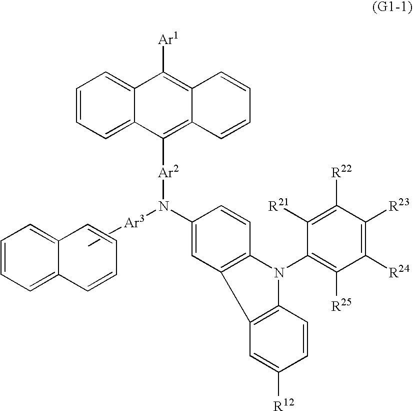
(In General Formula (G1-1), Ar¹ represents a substituted or unsubstituted aryl group having 6 to 13 carbon atoms; Ar² and Ar³ each independently represent a substituted or unsubstituted arylene group having 6 to 13 carbon atoms; R¹² represents hydrogen, an alkyl group having 1 to 4 carbon atoms, or a substituted or unsubstituted aryl group having 6 to 13 carbon atoms; and R²¹ to R²⁵ each represent hydrogen, an alkyl group having 1 to 4 carbon atoms, or a substituted or unsubstituted aryl group having 6 to 10 carbon atoms.)

3. An anthracene derivative represented by following General Formula (G1-2). 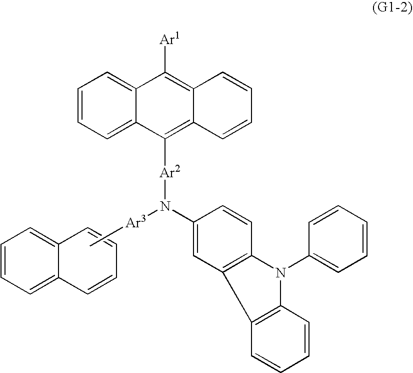
(In General Formula (G1-2), Ar¹ represents a substituted or unsubstituted phenyl group, a 1-naphthyl group, or a 2-naphthyl group; and Ar² and Ar³ each independently represent a substituted or unsubstituted arylene group having 6 to 13 carbon atoms.)

4. An anthracene derivative represented by following General Formula (G1-3). 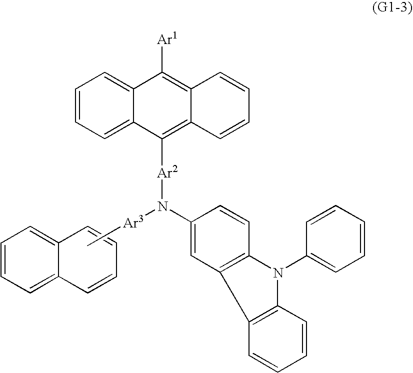
(In General Formula (G1-3), Ar¹ represents a substituted or unsubstituted phenyl group, a 1-naphthyl group, or a 2-naphthyl group; Ar² represents a substituted or unsubstituted arylene group having 6 to 13 carbon atoms; and Ar³ represents a para-phenylene group or a biphenyl-4,4′-diyl group.)

5. An anthracene derivative represented by following General Formula (G1-4). 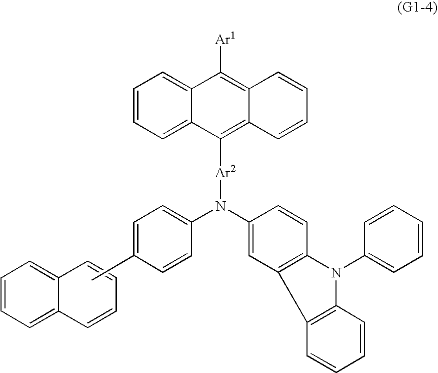
(In General Formula (G1-4), Ar¹ represents a substituted or unsubstituted phenyl group, a 1-naphthyl group, or a 2-naphthyl group; and Ar² represents a para-phenylene group or a biphenyl-4,4′-diyl group.)

6. An anthracene derivative represented by following Structural Formula (101).

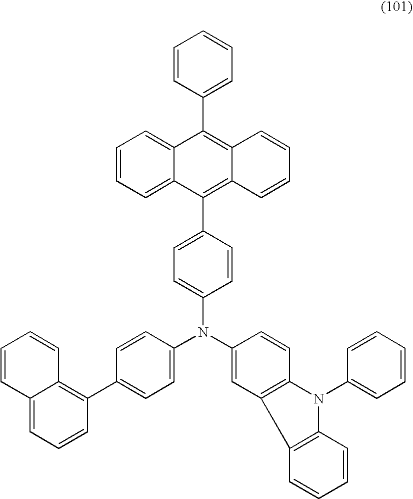

7. An anthracene derivative represented by following General Formula (G2). 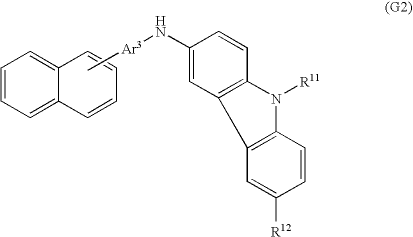
(In General Formula (G2), Ar³ represents a substituted or unsubstituted arylene group having 6 to 13 carbon atoms; R¹¹ represents either an alkyl group having 1 to 4 carbon atoms or a substituted or unsubstituted aryl group having 6 to 13 carbon atoms; and R¹² represents hydrogen, an alkyl group having 1 to 4 carbon atoms, or a substituted or unsubstituted aryl group having 6 to 13 carbon atoms.)

8. An anthracene derivative represented by following General Formula (G2-1). 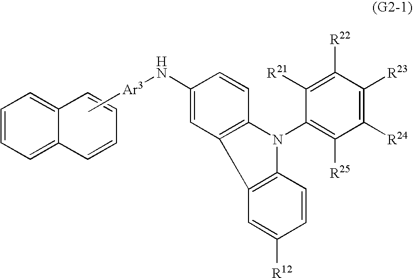
(In General Formula (G2-1), Ar³ represents a substituted or unsubstituted arylene group having 6 to 13 carbon atoms; R¹² represents hydrogen, an alkyl group having 1 to 4 carbon atoms, or a substituted or unsubstituted aryl group having 6 to 13 carbon atoms; and R²¹ to R²⁵ each represent hydrogen, an alkyl group having 1 to 4 carbon atoms, or a substituted or unsubstituted aryl group having 6 to 10 carbon atoms.)

9. An anthracene derivative represented by following General Formula (G2-2). 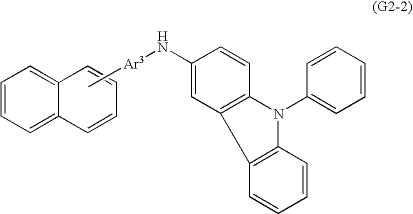
(In General Formula (G2-2), Ar³ represents a substituted or unsubstituted arylene group having 6 to 13 carbon atoms.)

10. An anthracene derivative represented by following General Formula (G2-3). 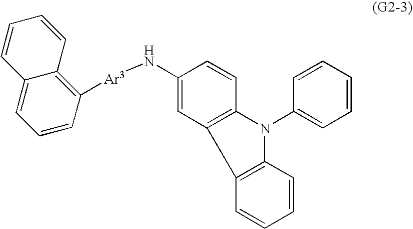
(In General Formula (G2-3), Ar³ represents a biphenyl-4,4′-diyl group.)

11. An anthracene derivative represented by following General Formula (G2-4).

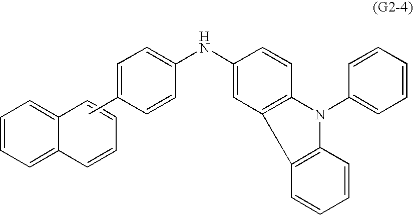

12. An anthracene derivative represented by following Structural Formula (301).

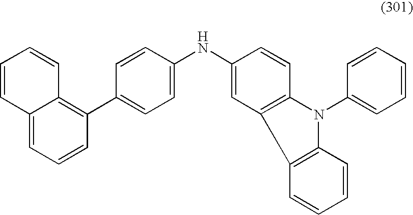

13. A light-emitting element comprising:
the anthracene derivative according to claim 1 between a pair of electrodes.

14. A light-emitting element comprising:
the anthracene derivative according to claim 2 between a pair of electrodes.

15. A light-emitting element comprising:
the anthracene derivative according to claim 3 between a pair of electrodes.

16. A light-emitting element comprising:
the anthracene derivative according to claim 4 between a pair of electrodes.

17. A light-emitting element comprising:
the anthracene derivative according to claim 5 between a pair of electrodes.

18. A light-emitting element comprising:
the anthracene derivative according to claim 6 between a pair of electrodes.

19. A light-emitting element comprising:
a light-emitting layer between a pair of electrodes, the light-emitting layer comprising:
the anthracene derivative according to claim 1.

20. A light-emitting element comprising:
a light-emitting layer between a pair of electrodes, the light-emitting layer comprising:
the anthracene derivative according to claim 2.

21. A light-emitting element comprising:
a light-emitting layer between a pair of electrodes, the light-emitting layer comprising:
the anthracene derivative according to claim 3.

22. A light-emitting element comprising:
a light-emitting layer between a pair of electrodes, the light-emitting layer comprising:
the anthracene derivative according to claim 4.

23. A light-emitting element comprising:
a light-emitting layer between a pair of electrodes, the light-emitting layer comprising:
the anthracene derivative according to claim 5.

24. A light-emitting element comprising:
a light-emitting layer between a pair of electrodes, the light-emitting layer comprising:
the anthracene derivative according to claim 6.

25. A light-emitting device comprising:
the light-emitting element according to claim 13; and
a control circuit which controls light emission of the light emitting element.

26. A light-emitting device comprising:
the light-emitting element according to claim 14; and
a control circuit which controls light emission of the light emitting element.

27. A light-emitting device comprising:
the light-emitting element according to claim 15; and
a control circuit which controls light emission of the light emitting element.

28. A light-emitting device comprising:
the light-emitting element according to claim 16; and
a control circuit which controls light emission of the light emitting element.

29. A light-emitting device comprising:
the light-emitting element according to claim 17; and
a control circuit which controls light emission of the light emitting element.

30. A light-emitting device comprising:
the light-emitting element according to claim 18; and
a control circuit which controls light emission of the light emitting element.

31. A light-emitting device comprising:
the light-emitting element according to claim 19; and
a control circuit which controls Eight emission of the light emitting element.

32. A light-emitting device comprising:
the light-emitting element according to claim 20; and
a control circuit which controls light emission of the light emitting element.

33. A light-emitting device comprising:
the light-emitting element according to claim 21; and
a control circuit which controls light emission of the light emitting element.

34. A light-emitting device comprising:
the light-emitting element according to claim 22; and
a control circuit which controls light emission of the light emitting element.

35. A light-emitting device comprising:
the light-emitting element according to claim 23; and
a control circuit which controls light emission of the light emitting element.

36. A light-emitting device comprising:
the light-emitting element according to claim 24; and
a control circuit which controls light emission of the light emitting element.

37. An electronic device comprising:
a display portion, the display portion comprising:
the light-emitting element according to claim 13 and a control circuit which controls light emission of the light emitting element.

38. An electronic device comprising:
a display portion, the display portion comprising:
the light-emitting element according to claim 14 and a control circuit which controls light emission of the light emitting element.

39. An electronic device comprising:
a display portion, the display portion comprising:
the light-emitting element according to claim 15 and a control circuit which controls light emission of the light emitting element.

40. An electronic device comprising:
a display portion, the display portion comprising:
the light-emitting element according to claim 16 and a control circuit which controls light emission of the light emitting element.

41. An electronic device comprising:
a display portion, the display portion comprising:
the light-emitting element according to claim 17 and a control circuit which controls light emission of the light emitting element.

42. An electronic device comprising:
a display portion, the display portion comprising:
the light-emitting element according to claim 18 and a control circuit which controls light emission of the light emitting element.

43. An electronic device comprising:
a display portion, the display portion comprising:
the light-emitting element according to claim 19 and a control circuit which controls light emission of the light emitting element.

44. An electronic device comprising:
a display portion, the display portion comprising:
the light-emitting element according to claim 20 and a control circuit which controls light emission of the light emitting element.

45. An electronic device comprising:
a display portion, the display portion comprising:
the light-emitting element according to claim 21 and a control circuit which controls light emission of the light emitting element.

46. An electronic device comprising:
a display portion, the display portion comprising:
the light-emitting element according to claim 22 and a control circuit which controls light emission of the light emitting element.

47. An electronic device comprising:
a display portion, the display portion comprising:
the light-emitting element according to claim 23 and a control circuit which controls light emission of the light emitting element.

48. An electronic device comprising:
a display portion, the display portion comprising:
the light-emitting element according to claim 24 and a control circuit which controls light emission of the light emitting element.

49. A lighting device applying the light-emitting device according to claim 25.

50. A lighting device applying the light-emitting device according to claim 26.

51. A lighting device applying the light-emitting device according to claim 27.

52. A lighting device applying the light-emitting device according to claim 28.

53. A lighting device applying the light-emitting device according to claim 29.

54. A lighting device applying the light-emitting device according to claim 30.

55. A lighting device applying the light-emitting device according to claim 31.

56. A lighting device applying the light-emitting device according to claim 32.

57. A lighting device applying the light-emitting device according to claim 33.

58. A lighting device applying the light-emitting device according to claim 34.

59. A lighting device applying the light-emitting device according to claim 35.

60. A lighting device applying the light-emitting device according to claim 36.
